(19) United States
(12) Patent Application Publication
(10) Pub. No.: US 2019/0010231 A1
(43) Pub. Date: Jan. 10, 2019

(54) NOVEL FUSION POLYPEPTIDE SPECIFIC FOR LAG-3 AND PD-1

(71) Applicant: Pieris Pharmaceuticals GmbH, Freising-Weihenstephan (DE)

(72) Inventors: Christine ROTHE, Dachau (DE); Rachida Siham BEL AIBA, Munich (DE); Shane OLWILL, Freising (DE); Sven BERGER, Munich (DE)

(73) Assignee: Pieris Pharmaceuticals GmbH, Freising-Weihenstephan (DE)

(21) Appl. No.: 15/750,651

(22) PCT Filed: Aug. 8, 2016
(86) PCT No.: PCT/EP2016/068860

§ 371 Date: Feb. 6, 2018

(30) Foreign Application Priority Data
Aug. 7, 2015 (EP) 15180116.4
Jan. 11, 2016 (EP) 16150707.4

(51) Int. Cl. C07K 16/28; C07K 14/47; A61P 35/00; C12N 15/62

(52) U.S. Cl. CPC C07K 16/2818; C07K 14/47; A61K 38/03; C12N 15/62; A61P 35/00

(57) ABSTRACT

The disclosure provides a fusion polypeptide specific for both immune checkpoints PD-1 and LAG-3, whereby the fusion polypeptide can be useful for generating a durable anti-tumor or anti-infection response. Such fusion polypeptide can be used in many pharmaceutical applications, for example, as anti-cancer agents and/or immune modulators for the treatment or prevention of human diseases such as a variety of tumors, or as anti-infection agents. The present disclosure also concerns methods of making the fusion polypeptide described herein as well as compositions comprising such fusion polypeptide. The present disclosure further relates to nucleic acid molecules encoding such fusion polypeptide and to methods for generation of such fusion polypeptide and nucleic acid molecules. In addition, the application discloses therapeutic and/or diagnostic uses of such fusion polypeptide as well as compositions comprising one or more of such fusion polypeptides.

### I. BACKGROUND

Lymphocyte activation gene-3, or LAG-3 (also known as cluster of differentiation 223 or CD223) is a membrane protein of the immunoglobulin supergene family. LAG-3 is structurally and genetically related to cluster of differentiation 4 (CD4), with its encoding gene located on the distal part of the short arm of chromosome 12, near the CD4 gene, suggesting that the LAG-3 gene may have evolved through gene duplication (Triebel et al., J Exp Med, 1990). LAG-3 is not expressed on resting peripheral blood lymphocytes but is expressed on activated T cells and natural killer (NK) cells (Triebel et al., J Exp Med, 1990), and has been reported to also be expressed on activated B cells (Kisielow et al., Eur J Immunol, 2005) and plasmacytoid dendritic cells (Workman et al., J Immunol, 2009).

Like CD4, LAG-3 binds to major histocompatibility complex (MHC) class II molecules, but with a two-fold higher affinity and at a different binding site than CD4 (Huard et al., Proc Natl Acad Sci, 1997). MHC class II engagement on dendritic cells by LAG-3 leads to changes in the cytokine and chemokine profiles of dendritic cells (Buisson and Triebel, Vaccine, 2003). Further, LAG-3 has been reported to cause maturation of dendritic cells, as demonstrated by the production of interleukin 12 (IL-12) and tissue necrosis factor alpha (TNF-α) by these cells and increases in the capacity of dendritic cells to stimulate the proliferation and interferon gamma (IFN-γ) response by allogeneic T cells (Andreae et al., J Immunol, 2002). LAG-3 signaling and MHC class II cross-linking has been reported to inhibit early events in primary activation of human cluster of differentiation 4 positive (CD4⁺) and cluster of differentiation 8 positive (CD8⁺) T cells (Macon-Lemaitre and Triebel, Immunology, 2005). LAG-3 negatively regulates the cellular proliferation, activation and homeostasis of T cells.

Therefore, like cytotoxic T-lymphocyte-associated protein 4 (CTLA-4) and programmed cell death protein 1 (PD-1), LAG-3 is an inhibitory immune receptor. LAG-3's prominent role as a negative regulator of T cell response has been impressively demonstrated, in particular in conjunction with PD-1 in a study based on both knockout mice and target-specific antibodies (Woo et al., Cancer Res, 2012). In that study, dual anti-LAG-3/anti-PD-1 antibody treatment cured most mice of established tumors that were largely resistant to single antibody treatment. Further, LAG-3/PD-1 double knock-out mice showed markedly increased survival from and clearance of multiple transplantable tumors. Additional experimental support for the powerful combined role of PD-1 and LAG-3 as inhibitory immune checkpoints was provided by the fact that the double knock-out mice were highly prone to lethal autoinflammation.

Programmed cell death protein 1, or PD-1 (also known as cluster of differentiation 279 or CD279) is a member of the cluster of differentiation 28 (CD28) gene family and is expressed on activated T, B, and myeloid lineage cells (Sharpe et al., Nat Immunol, 2007, Greenwald et al., Annu Rev Immunol, 2005). PD-1 interacts with two ligands, programmed cell death 1 ligand 1 (PD-L1) and programmed cell death 1 ligand 2 (PD-L2). Interaction of these ligands with PD-1 plays an important role in downregulating the immune system by limiting overly-active T cells locally, which in turn prevents autoimmunity and maintains peripheral tolerance during infection or inflammation in normal tissues.

PD-1 negatively modulates T cell activation, and the inhibitory function of PD-1 on T cell activation is linked to an immunoreceptor tyrosine-based inhibitory motif (ITIM) of its cytoplasmic domain (Greenwald et al., Annu Rev Immunol, 2005, Parry et al., Mol Cell Biol, 2005). Disruption of this inhibitory function of PD-1 can lead to autoimmunity. On the other hand, sustained negative signals by PD-1 have been implicated in T cell dysfunctions in many pathologic situations, such as chronic viral infections and tumor immune evasion.

In many cancers, PD-1 is expressed by tumor-infiltrating lymphocytes (TILs), associated with host anti-tumor immunity (Galon et al., Science, 2006). Multiple lines of evidence have indicated that TILs are subject to PD-1 inhibitory regulation and the anti-tumor immunity is modulated by PD-1/PD-L1 signaling. First, PD-L1 expression is confirmed in many human and mouse tumor lines and the expression can be further upregulated by IFN-γ in vitro (Dong et al., Nat Med, 2002). Second, expression of PD-L1 by tumor cells has been directly associated with their resistance to lysis by anti-tumor T cells in vitro (Dong et al., Nat Med, 2002, Blank et al., Cancer Res, 2004). Third, PD-1 knockout mice are resistant to tumor challenge (Iwai et al., Int Immunol, 2005) and T cells from PD-1 knockout mice are highly effective in tumor rejection when adoptively transferred to tumor-bearing mice (Blank et al., Cancer Res, 2004). Fourth, blocking PD-1 inhibitory signals by a monoclonal antibody can potentiate host anti-tumor immunity in mice (Iwai et al., Int Immunol, 2005, Hirano et al., Cancer Res, 2005). Fifth, high degrees of PD-L1 expression in tumors (detected by immunohistochemical staining) are associated with poor prognosis for many human cancer types (Hamanishi et al., Proc Natl Acad Sci USA, 2007).

There thus exists a need for new compounds that can modulate responses of LAG-3⁺ lymphocytes, such as T cells, NK cells, B cells, and plasmacytoid dendritic cells, and at the same time, relieve such lymphocytes of PD-1 inhibitory regulation. Such combination may have important uses in the treatment or prevention of cancer, organ transplant rejection, or treatment of autoimmune or autoinflammatory diseases. In this regard, the present disclosure provides a group of novel proteins binding to both LAG-3 and PD-1, thereby, modulating the immune response.

### II. DEFINITIONS

The following list defines terms, phrases, and abbreviations used throughout the instant specification. All terms listed and defined herein are intended to encompass all grammatical forms.

As used herein, unless otherwise specified, “LAG-3” means human LAG-3 (huLAG-3) and include variants, isoforms and species homologs of human LAG-3. LAG-3 is also known as “lymphocyte-activation gene 3”, “cluster of differentiation 223”, or “CD223”, which are used interchangeably. Human LAG-3 means a full-length protein defined by UniProt P18627 (version 5 of 7 Jul. 2009), a fragment thereof, or a variant thereof. Human LAG-3 is encoded by the LAG3 gene.

As used herein, unless otherwise specified, “PD-1” means human PD-1 (hPD-1) and includes variants, isoforms and species homologs of human PD-1. PD-1 is also known as “programmed cell death protein 1”, “cluster of differentiation 279” or “CD279”, which are used interchangeably. Human PD-1 means a full-length protein defined by UniProt Q15116, a fragment thereof, or a variant thereof. Human PD-1 is encoded by the PDCD1 gene.

As used herein, “detectable affinity” means the ability to bind to a selected target with an affinity constant, generally measured by K_{d} or EC₅₀, of at most about 10⁻⁵ M or below (a lower K_{d} or EC₅₀ value reflects better binding activity). Lower affinities that are no longer measurable with common methods such as ELISA (enzyme-linked immunosorbent assay) are of secondary importance.

As used herein, “binding affinity” of a protein of the disclosure (e.g. a lipocalin mutein or an antibody) or a fusion polypeptide thereof to one or more selected targets (in the present case, LAG-3 and/or PD-1), can be measured (and thereby K_{d} values of a mutein-ligand complex be determined) by a multitude of methods known to those skilled in the art. Such methods include, but are not limited to, fluorescence titration, competitive ELISA, calorimetric methods, such as isothermal titration calorimetry (ITC), and surface plasmon resonance (SPR). Such methods are well established in the art and examples thereof are also detailed below.

It is also noted that the complex formation between the respective binder and its ligand is influenced by many different factors such as the concentrations of the respective binding partners, the presence of competitors, pH and the ionic strength of the buffer system used, and the experimental method used for determination of the dissociation constant K_{d} (for example fluorescence titration, competition ELISA or surface plasmon resonance, just to name a few) or even the mathematical algorithm which is used for evaluation of the experimental data.

Therefore, it is also clear to the skilled person that the K_{d} values (dissociation constant of the complex formed between the respective binder and its target/ligand) may vary within a certain experimental range, depending on the method and experimental setup that is used for determining the affinity of a particular lipocalin mutein for a given ligand. This means that there may be a slight deviation in the measured K_{d} values or a tolerance range depending, for example, on whether the K_{d} value was determined by surface plasmon resonance (SPR), by competitive ELISA, by direct ELISA, or by another method.

As used herein, a “mutein,” a “mutated” entity (whether protein or nucleic acid), or “mutant” refers to the exchange, deletion, or insertion of one or more nucleotides or amino acids, compared to the naturally occurring (wild-type) nucleic acid or protein “reference” scaffold. Said term also includes fragments of a mutein and variants as described herein. Lipocalin muteins of the present disclosure, fragments or variants thereof preferably have the function of binding to LAG-3 as described herein.

The term “fragment” as used herein in connection with the muteins of the disclosure relates to proteins or peptides derived from full-length mature human tear lipocalin (hTlc or hTLPC) that are N-terminally and/or C-terminally shortened, i.e. lacking at least one of the N-terminal and/or C-terminal amino acids. Such a fragment may lack up to 2, up to 3, up to 4, up to 5, up to 10, up to 15, up to 20, up to 25, or up to 30 (including all numbers in between) of the N-terminal and/or C-terminal amino acids. As an illustrative example, such a fragment may lack 4 N-terminal and 2 C-terminal amino acids. It is understood that the fragment is preferably a functional fragment of the full-length tear lipocalin (mutein), which means that it preferably comprises the binding pocket of the full length tear lipocalin (mutein) it is derived from. As an illustrative example, such a functional fragment may comprise at least amino acids 7-153 of the linear polypeptide sequence of native mature hTlc. Such fragments may include at least 10, more such as 20 or 30 or more consecutive amino acids of the primary sequence of the mature lipocalin and are usually detectable in an immunoassay of the mature lipocalin. In general, the term “fragment,” as used herein with respect to the corresponding protein ligand LAG-3 of a lipocalin mutein of the disclosure or of the combination according to the disclosure or of a fusion protein described herein, relates to N-terminally and/or C-terminally shortened protein or peptide ligands, which retain the capability of the full length ligand to be recognized and/or bound by a mutein according to the disclosure.

The term “mutagenesis” as used herein means that the experimental conditions are chosen such that the amino acid naturally occurring at a given sequence position of the mature lipocalin can be substituted by at least one amino acid that is not present at this specific position in the respective natural polypeptide sequence. The term “mutagenesis” also includes the (additional) modification of the length of sequence segments by deletion or insertion of one or more amino acids. Thus, it is within the scope of the disclosure that, for example, one amino acid at a chosen sequence position is replaced by a stretch of three random mutations, leading to an insertion of two amino acid residues compared to the length of the respective segment of the wild-type protein. Such an insertion or deletion may be introduced independently from each other in any of the peptide segments that can be subjected to mutagenesis in the disclosure. In one exemplary embodiment of the disclosure, an insertion of several mutations may be introduced into the loop AB of the chosen lipocalin scaffold (cf. International Patent Publication No. WO 2005/019256, which is incorporated by reference its entirety herein).

The term “random mutagenesis” means that no predetermined single amino acid (mutation) is present at a certain sequence position but that at least two amino acids can be incorporated with a certain probability at a predefined sequence position during mutagenesis.

“Identity” is a property of sequences that measures their similarity or relationship. The term “sequence identity” or “identity” as used in the present disclosure means the percentage of pair-wise identical residues—following (homologous) alignment of a sequence of a polypeptide of the disclosure with a sequence in question—with respect to the number of residues in the longer of these two sequences. Sequence identity is measured by dividing the number of identical amino acid residues by the total number of residues and multiplying the product by 100.

The term “homology” is used herein in its usual meaning and includes identical amino acids as well as amino acids which are regarded to be conservative substitutions (for example, exchange of a glutamate residue by an aspartate residue) at equivalent positions in the linear amino acid sequence of a polypeptide of the disclosure (e.g., any lipocalin mutein of the disclosure).

The percentage of sequence homology or sequence identity can, for example, be determined herein using the program BLASTP, version blastp 2.2.5 (Nov. 16, 2002) (cf. Altschul et al., Nucleic Acids Res, 1997). In this embodiment the percentage of homology is based on the alignment of the entire polypeptide sequences (matrix: BLOSUM 62; gap costs: 11.1; cut-off value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

Specifically, in order to determine whether an amino acid residue of the amino acid sequence of a lipocalin (mutein) is different from a wild-type lipocalin corresponding to a certain position in the amino acid sequence of a wild-type lipocalin, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments. Accordingly, a wild-type sequence of lipocalin can serve as “subject sequence” or “reference sequence”, while the amino acid sequence of a lipocalin different from the wild-type lipocalin described herein serves as “query sequence”. The terms “wild-type sequence” and “reference sequence” and “subject sequence” are used interchangeably herein. A preferred wild-type sequence of lipocalin is the sequence of hTlc as shown in SEQ ID NO: 1.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity. Those skilled in the art will recognize that several computer programs are available for determining sequence identity using standard parameters, for example BLAST (Altschul et al., Nucleic Acids Res, 1997), BLAST2 (Altschul et al., J Mol Biol, 1990), and Smith-Waterman (Smith and Waterman, J Mol Biol, 1981).

The term “variant” as used in the present disclosure relates to derivatives of a protein or peptide that include modifications of the amino acid sequence, for example by substitution, deletion, insertion or chemical modification. Such modifications do in some embodiments not reduce the functionality of the protein or peptide. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline. However, such substitutions may also be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. The term “variant,” as used herein with respect to the corresponding protein target LAG-3 and/or PD-1 of a lipocalin mutein of the disclosure or of a combination and/or fusion protein according to the disclosure, relates to LAG-3 and/or PD-1 or fragment thereof, respectively, that has one or more such as 1, 2, 3, 4, 5, 6, 7, 8, 9, 10, 12, 14, 16, 18, 20, 22, 24, 26, 28, 30, 40, 50, 60, 70, 80 or more amino acid substitutions, deletions and/or insertions in comparison to a wild-type LAG-3 or PD-1 protein, respectively, such as a LAG-3 or PD-1 reference protein as deposited with SwissProt/UniProt as described herein. A LAG-3 or PD-1 variant, respectively, has preferably an amino acid identity of at least 50%, 60%, 70%, 80%, 85%, 90% or 95% with a wild-type human LAG-3 or PD-1, such as a LAG-3 or PD-1 reference protein as deposited with SwissProt/UniProt as described herein.

By a “native sequence” of a lipocalin is meant that the sequence of a lipocalin that has the same amino acid sequence as the corresponding polypeptide derived from nature. Thus, a native sequence lipocalin can have the amino acid sequence of the respective naturally-occurring lipocalin from any organism, in particular a mammal. Such native sequence polypeptide can be isolated from nature or can be produced by recombinant or synthetic means. The term “native sequence” polypeptide specifically encompasses naturally-occurring truncated or secreted forms of the lipocalin, naturally-occurring variant forms such as alternatively spliced forms and naturally-occurring allelic variants of the lipocalin. A polypeptide “variant” means a biologically active polypeptide having at least about 50%, 60%, 70%, 80% or at least about 85% amino acid sequence identity with the native sequence polypeptide. Such variants include, for instance, polypeptides in which one or more amino acid residues are added or deleted at the N- or C-terminus of the polypeptide. Generally, a variant has at least about 70%, including at least about 80%, such as at least about 85% amino acid sequence identity, including at least about 90% amino acid sequence identity or at least about 95% amino acid sequence identity with the native sequence polypeptide. As an illustrative example, the first four N-terminal amino acid residues (His-His-Leu-Leu) and the last 2 C-terminal amino acid residues (Ser-Asp) can be deleted in a hTlc mutein of the disclosure without affecting the biological function of the protein, e.g. SEQ ID NOs: 13-28.

The term “position” when used in accordance with the disclosure means the position of either an amino acid within an amino acid sequence depicted herein or the position of a nucleotide within a nucleic acid sequence depicted herein. To understand the term “correspond” or “corresponding” as used herein in the context of the amino acid sequence positions of one or more lipocalin muteins, a corresponding position is not only determined by the number of the preceding nucleotides/amino acids. Accordingly, the position of a given amino acid in accordance with the disclosure which may be substituted may vary due to deletion or addition of amino acids elsewhere in a (mutant or wild-type) lipocalin. Similarly, the position of a given nucleotide in accordance with the present disclosure which may be substituted may vary due to deletions or additional nucleotides elsewhere in a mutein or wild-type lipocalin 5′-untranslated region (UTR) including the promoter and/or any other regulatory sequences or gene (including exons and introns).

Thus, for a “corresponding position” in accordance with the disclosure, it is preferably to be understood that the positions of nucleotides/amino acids may differ in the indicated number than similar neighboring nucleotides/amino acids, but said neighboring nucleotides/amino acids, which may be exchanged, deleted, or added, are also comprised by the one or more “corresponding positions”.

In addition, for a corresponding position in a lipocalin mutein based on a reference sequence in accordance with the disclosure, it is preferably understood that the positions of nucleotides/amino acids structurally correspond to the positions elsewhere in a (mutant or wild-type) lipocalin, even if they may differ in the indicated number, as appreciated by the skilled in light of the highly-conserved overall folding pattern among lipocalins.

The term “albumin” includes all mammal albumins such as human serum albumin or bovine serum albumin or rat serum albumin.

The term “organic molecule” or “small organic molecule” as used herein for the non-natural target denotes an organic molecule comprising at least two carbon atoms, but preferably not more than 7 or 12 rotatable carbon bonds, having a molecular weight in the range between 100 and 2,000 Dalton, preferably between 100 and 1,000 Dalton, and optionally including one or two metal atoms.

The word “detect”, “detection”, “detectable”, or “detecting” as used herein is understood both on a quantitative and a qualitative level, as well as a combination thereof. It thus includes quantitative, semi-quantitative and qualitative measurements of a molecule of interest.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, and etc., to name only a few illustrative examples. Preferably, the “mammal” herein is human.

An “effective amount” is an amount sufficient to effect beneficial or desired results. An effective amount can be administered in one or more administrations.

A “sample” is defined as a biological sample taken from any subject. Biological samples include, but are not limited to, blood, serum, urine, feces, semen, or tissue.

A “subunit” of a fusion polypeptide disclosed herein is defined as a stretch of amino acids of the polypeptide, which stretch defines a unique functional unit of said polypeptide such as provides binding motif towards a target.

A “fusion polypeptide” as described herein comprises two or more subunits, at least one of these subunits binds to LAG-3 and a further subunit binds to PD-1. Within the fusion polypeptide, these subunits may be linked by covalent or non-covalent linkage. Preferably, the fusion polypeptide is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion polypeptide of the present disclosure may also be linked by a chemical linker.

A “linker” that may be comprised by a fusion polypeptide of the present disclosure links two or more subunits of a fusion polypeptide as described herein. The linkage can be covalent or non-covalent. A preferred covalent linkage is via a peptide bond, such as a peptide bond between amino acids. Accordingly, in a preferred embodiment said linker comprises of one or more amino acids, such as 1, 2, 3, 4, 5, 6, 7, 8, 9, 10, 11, 12, 13, 14, 15, 16, 17, 18, 19, 20 or more amino acids. Preferred linkers are described herein. Other preferred linkers are chemical linkers.

### III. DESCRIPTIONS OF FIGURES

FIG. 1: provides an overview of the design of representative fusion polypeptides described in this application that are bispecific for the targets PD-1 and LAG-3, or monospecific for LAG-3. Representative bispecific fusion polypeptides of FIG. 1 a-e were made based on an antibody specific for PD-1 (e.g. the antibody of SEQ ID NOs: 3 and 4) and one or more lipocalin muteins specific for LAG-3 (e.g. the lipocalin mutein of SEQ ID NO: 17 or the lipocalin mutein of SEQ ID NO: 27). The lipocalin muteins were genetically fused to either the C- or the N-terminus of either the heavy chain or the light chain of the PD-1 specific antibody as depicted in FIG. 1, resulting in the fusion polypeptides of SEQ ID NOs: 5 and 4, SEQ ID NOs: 9 and 4, SEQ ID NOs: 6 and 4, SEQ ID NOs: 10 and 4, SEQ ID NOs: 3 and 7, SEQ ID NOs: 3 and 11, SEQ ID NOs: 3 and 8, and SEQ ID NOs: 3 and 12. LAG-3 monospecific fusion polypeptides were made by genetically fusing the lipocalin mutein of SEQ ID NO: 17 or the lipocalin mutein of SEQ ID NO: 27 to the C-terminus of the Fc portion of SEQ ID NO: 3, resulting in SEQ ID NO: 41 and SEQ ID NO: 42, respectively. FIG. 1 a-e shows additional representative fusion polypeptides that may be made using a different antibody specific for PD-1 (e.g. the antibody of SEQ ID NOs: 47 and 48) and one or more lipocalin muteins specific for LAG-3 (e.g. the lipocalin mutein of SEQ ID NO: 17 or the lipocalin mutein of SEQ ID NO: 27). The lipocalin muteins may be genetically fused to either the C- or the N-terminus of either the heavy chain or the light chain of the PD-1 specific antibody as depicted in FIG. 1 to yield the fusion polypeptides of SEQ ID NOs: 51 and 48, SEQ ID NOs: 55 and 48, SEQ ID NOs: 52 and 48, SEQ ID NOs: 56 and 48, SEQ ID NOs: 47 and 53, SEQ ID NOs: 47 and 57, SEQ ID NOs: 47 and 54, and SEQ ID NOs: 47 and 58. FIG. 1 f-i additionally shows the design of additional fusion polypeptides and corresponding sequences for such polypeptides where made based on an antibody specific for PD-1 (e.g. the antibody of SEQ ID NOs: 3 and 4 or the antibody of SEQ ID NOs: 47 and 48) and one or more lipocalin muteins specific for LAG-3 (e.g. the lipocalin mutein of SEQ ID NO: 17 or the lipocalin mutein of SEQ ID NO: 27).

FIG. 2: depicts the results of an enzyme-linked immunosorbent assay (ELISA) in which the binding to PD-1 of representative fusion polypeptides, the benchmark antibody (SEQ ID NOs: 3 and 4), and a negative control lipocalin mutein (SEQ ID NO: 43) was determined. Recombinant human PD-1-His (PD-1 with a C-terminal polyhistidine tag) was coated on a microtiter plate, and the tested agents (fusion polypeptides, benchmark antibody (SEQ ID NOs: 3 and 4), and negative control lipocalin mutein (SEQ ID NO: 43)) were titrated starting with the highest concentration of 250 nM and a 1:3 dilution series. Bound samples under study were detected via an anti-human IgG Fc antibody as described in Example 2. The data was fit with a 1:1 binding model with EC₅₀ value and the maximum signal as free parameters, and a slope that was fixed to one. FIG. 2A shows results for fusion polypeptides with lipocalin mutein of SEQ ID NO: 17 and FIG. 2B shows results for fusion polypeptides with lipocalin mutein of SEQ ID NO: 27. The resulting EC₅₀ values are provided in Table 2.

FIG. 3: shows the results of an ELISA experiment in which the binding to LAG-3 of representative fusion polypeptides, the benchmark antibody (SEQ ID NOs: 3 and 4), and the LAG-3-binding lipocalin muteins (SEQ ID NOs: 17 and 27) and the negative control lipocalin mutein that does not bind LAG-3 (SEQ ID NO: 43) was determined. Human LAG-3-His (LAG-3 with C-terminal polyhistidine tag) was coated on a microtiter plate, and the tested agents were titrated starting with the highest concentration of 250 nM. Bound agents under study were detected via an anti-Tic antibody or via an anti-human-IgG-Fc antibody as described in Example 3. The data was fit with a 1:1 binding model with EC₅₀ value and the maximum signal as free parameters, and a slope that was fixed to one. FIGS. 3A and 3C show results for fusion polypeptides with lipocalin mutein of SEQ ID NO: 17 detected with an anti-Tic antibody and anti-human-IgG-Fc antibody, respectively. FIGS. 3B and 3D show results for fusion polypeptides with lipocalin mutein of SEQ ID NO: 27, detected with an anti-Tlc antibody and anti-human-IgG-Fc antibody, respectively. The resulting EC₅₀ values are provided in Table 3.

FIG. 4: depicts the results of fluorescence-activated cell sorting (FACS) studies carried out in order to assess the specific binding of fusion polypeptides to human PD-1 (FIG. 4A) or human LAG-3 (FIG. 4B), respectively, expressed on mammalian cells as described in Example 4. The negative control combination of hIgG4 (Sigma) and SEQ ID NO: 43 showed no binding. The geometric means of the fluorescence intensity were normalized to maximal mean and fit with a 1:1 binding model. The resulting EC₅₀ values are provided in Table 4.

FIG. 5: illustrates the results of an ELISA experiment in which the ability of representative fusion polypeptides to simultaneously bind both targets, PD-1 and LAG-3, was determined. Recombinant PD-1-His was coated on a microtiter plate, followed by a titration of the fusion polypeptides starting with the highest concentration of 250 nM. Subsequently, a constant concentration of biotinylated human LAG-3-Fc was added, which was detected via extravidin as described in Example 5. FIG. 5A shows results for fusion polypeptides with the lipocalin mutein of SEQ ID NO: 17 and the benchmark antibody against PD-1 of SEQ ID NOs: 3 and 4 and FIG. 5B shows results for fusion polypeptides with the lipocalin mutein of SEQ ID NO: 27 and the benchmark antibody against PD-1 of SEQ ID NOs: 3 and 4.

FIG. 6: shows that the fusion polypeptides compete with major histocompatibility complex (MHC) class II molecules (LAG-3's natural ligands) for binding to LAG-3, depicted in competitive FACS studies conducted as described in Example 6. A constant concentration of human LAG-3-Fc fusion (human LAG-3 extracellular domain fused to human IgG1 Fc fragment), and a dilution series of fusion polypeptides or controls, were incubated with the MHC class II positive human cell line A375. Cell-bound huLAG-3-Fc was detected using a fluorescently labelled anti-IgG Fc antibody. The dose dependent inhibition of huLAG-3-Fc binding to MHC class II molecules by LAG-3 and PD-1 bispecific antibody-lipocalin mutein fusion polypeptides or LAG-3 monospecific Fc-lipocalin mutein fusion polypeptides were observed.

FIG. 7: shows the results of a representative experiment in which the ability of the fusion polypeptide of SEQ ID NOs: 5 and 4 to induce T cell activation was investigated. The benchmark antibody (SEQ ID NOs: 3 and 4), and a cocktail of the benchmark antibody (SEQ ID NOs: 3 and 4) and Fc-lipocalin mutein fusion polypeptide (SEQ ID NO: 41) were also tested. In the experiment, staphylococcal enterotoxin B (SEB) stimulated human peripheral blood mononuclear cells (PBMCs) were incubated with the fusion polypeptide or controls at two different concentrations. Levels of secreted interleukin 2 (IL-2), reflective of T cell activation, were determined by an electrochemoluminescence-based assay as readouts for T cell activation, as described in Example 7.

FIG. 8: shows the results of a representative experiment in which the ability of the fusion polypeptide of SEQ ID NOs: 5 and 4 to induce T cell activation was investigated. The benchmark antibody (SEQ ID NOs: 3 and 4), and a cocktail of the benchmark antibody (SEQ ID NOs: 3 and 4) and Fc-lipocalin mutein fusion polypeptide (SEQ ID NO: 41) were also tested. In the experiment, melanoma A375 cells were coated and allowed to adhere overnight. The next day, purified T cells, pre-treated with phytohemagglutinin (PHA), were incubated on the coated cells in the presence of various concentrations of the bispecific fusion polypeptide and the controls. Levels of supernatant interferon gamma (IFN-γ), reflective of T cell activation, were determined by an electrochemoluminescence-based assay, as described in Example 8.

### IV. DETAILED DESCRIPTION OF THE DISCLOSURE

In some embodiments, the fusion polypeptide contains at least two subunits in any order: (1) a first subunit that comprises a full-length immunoglobulin or an antigen-binding domain thereof specific for PD-1, and (2) a second subunit that comprises a lipocalin mutein specific for LAG-3.

In some embodiments, the fusion polypeptide also may contain a third subunit. For instance, the polypeptide may contain a third subunit specific for LAG-3. In some embodiments, said third subunit comprises a lipocalin mutein specific for LAG-3. For example, two lipocalin muteins may be fused to an immunoglobulin subunit, one at the C-terminus and one at the N-terminus of the immunoglobulin. In some embodiments, lipocalin muteins may be fused to the heavy chain or light chain of an immunoglobulin.

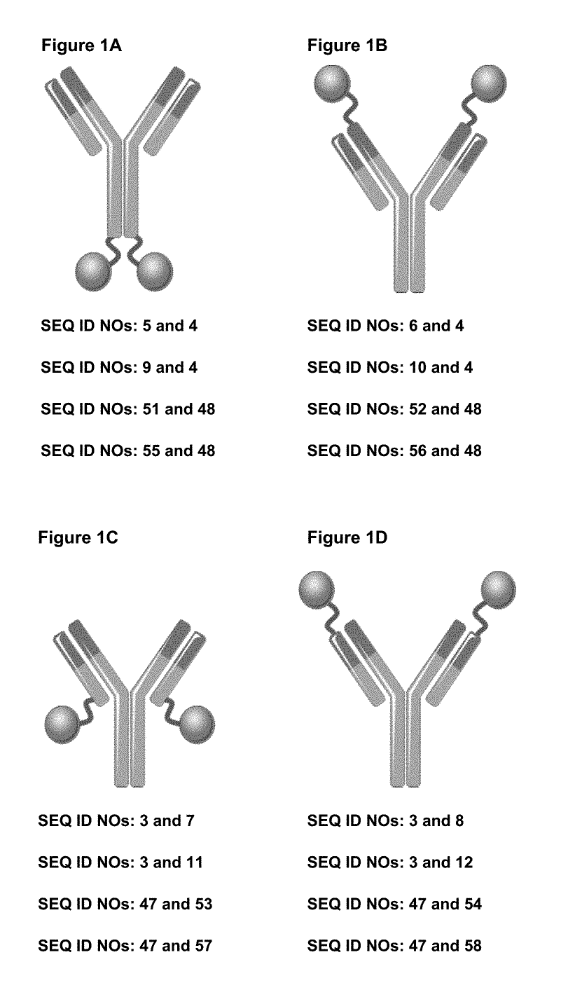

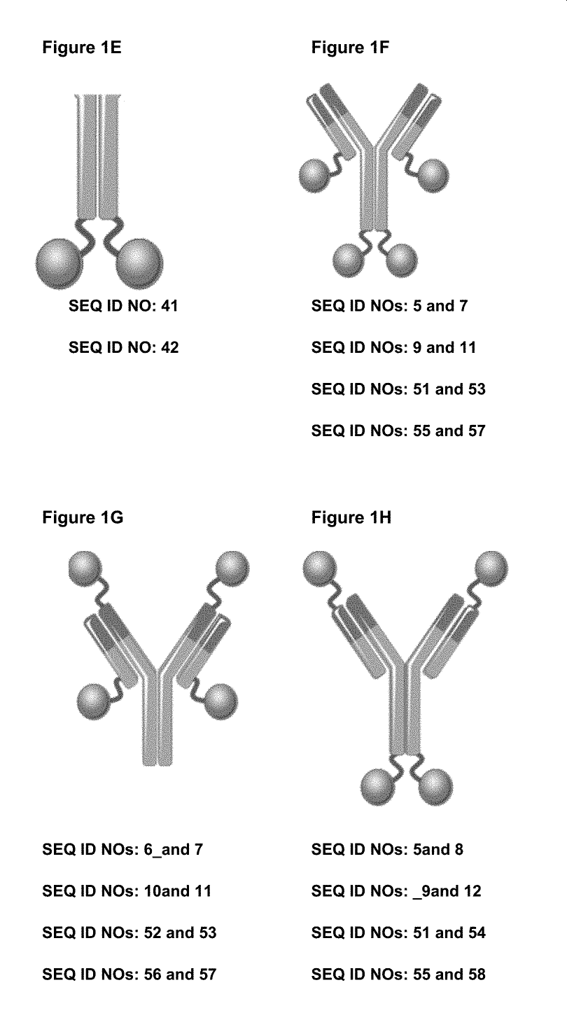

In some embodiments, one subunit can be linked to another subunit as essentially described in FIG. 1. For example, one lipocalin mutein can be linked, via a peptide bond, to the C-terminus of the immunoglobulin heavy chain domain (VH), the N-terminus of the VH, the C-terminus of the immunoglobulin light chain (VL), and/or the N-terminus of the VL (FIG. 1). In some particular embodiments, a lipocalin mutein subunit can be fused at its N-terminus and/or its C-terminus to an immunoglobulin subunit. For example, the lipocalin mutein may be linked via a peptide bond at the C-terminus of a heavy chain constant region (CH) or the C-terminus of a light chain constant region (CL) of the immunoglobulin. In some still further embodiments, the peptide bond may be a linker, preferably an unstructured (G₄S)₃ linker, for example, as shown in SEQ ID NO: 19. A linker may have from 1 to 50 amino acids, such as 1, 2, 3, 4, 5, 10, 11, 12, 13, 14, 15, 16, 17 18, 19, 20, 25, 30, 35, 40, 45 or 50 amino acids.

In this regard, one subunit may be fused at its N-terminus and/or its C-terminus to another subunit. For example, when one subunit comprises a full-length immunoglobulin, another subunit may be linked via a peptide bond between the N-terminus of the second subunit and the C-terminus of a heavy chain constant region (CH) of said immunoglobulin. In some further embodiments, a third subunit may be linked via a peptide bond between the N-terminus of the third binding domain and the C-terminus of a light chain constant region (CL) of said immunoglobulin. In some still further embodiments, the peptide bond may be a linker, preferably an unstructured (G₄S)₃ linker, for example, as shown in SEQ ID NO: 2.

In some embodiments with respect to a fusion polypeptide of the disclosure, one of whose subunits comprises a full-length immunoglobulin, while the polypeptide is simultaneously engaging PD-1 and LAG-3, the Fc function of the Fc region of the full-length immunoglobulin to Fc receptor-positive cell may be preserved at the same time.

In some other embodiments with respect to a fusion polypeptide of the disclosure, one of whose subunits comprises a full-length immunoglobulin, while the polypeptide is simultaneously engaging PD-1 and LAG-3, the Fc function of the Fc region of the full-length immunoglobulin to Fc receptor-positive cell may be reduced or fully suppressed by protein engineering. This may be achieved, for example, by switching from the IgG1 backbone to IgG4, as IgG4 is known to display reduced Fc-gamma receptor interactions compared to IgG1. To further reduce the residual binding to Fc-gamma receptors, mutations may be introduced into the IgG4 backbone such as F234A and L235A. In addition, a S228P mutation may be introduced into the IgG4 backbone to minimize the exchange of IgG4 half-antibody. In some still further embodiments, an additional N297A mutation may be present in the immunoglobulin heavy chain of the fusion polypeptide in order to remove the natural glycosylation motif.

In some embodiments, resulting from the simultaneous binding to PD-1 and LAG-3, the fusion polypeptides of the disclosure may exhibit a durable anti-tumor or anti-infection response.

In some embodiments, the Fc portion of the immunoglobulin included in a fusion polypeptide of the disclosure may contribute to maintaining the serum levels of the fusion polypeptide, critical for its stability and persistence in the body. For example, when the Fc portion binds to Fc receptors on endothelial cells and on phagocytes, the fusion polypeptide may become internalized and recycled back to the blood stream, enhancing its half-life within body.

In some embodiments, the fusion polypeptide may be able to bind PD-1 with an EC₅₀ value of at most about 10 nM or even lower, such as about 5 nM, about 1 nM, or about 0.5 nM or even lower, for example, when the fusion polypeptide is measured in an ELISA (enzyme-linked immunosorbent assay) assay essentially as described in Example 2.

In some embodiments, a fusion polypeptide of the disclosure may be able to bind PD-1 with an EC₅₀ value comparable to the EC₅₀ value of the immunoglobulin specific for PD-1 as included in such fusion polypeptide, such as the antibody having the heavy and light chains provided by SEQ ID NOs: 3 and 4, for example, when said immunoglobulin and the fusion polypeptide are measured in as ELISA assay essentially as described in Example 2.

In another aspect, the fusion polypeptide may be able to bind LAG-3 with an EC₅₀ value of at most about 10 nM or even lower, such as about 5 nM, about 1 nM or about 0.5 nM or even lower, for example, when the fusion polypeptide is measured in an ELISA assay essentially as described in Example 3.

In some embodiments, a fusion polypeptide of the disclosure may be able to bind LAG-3 with an EC₅₀ value at least as good as or superior to the EC₅₀ value of the lipocalin mutein specific for LAG-3 as included in such fusion polypeptide, such as the lipocalin mutein of SEQ ID NO: 17 or the lipocalin mutein of SEQ ID NO: 27, for example, when said lipocalin mutein and the polypeptide are measured in an ELISA assay essentially as described in Example 3.

In some embodiments, the fusion polypeptides of the disclosure specific for both PD-1 and LAG-3 may be capable of simultaneously binding of PD-1 and LAG-3, for example, when said fusion polypeptide is measured in an ELISA assay essentially described in Example 5. In some embodiments, the fusion polypeptide may be capable of simultaneously binding of PD-1 and LAG-3, with an EC₅₀ value of at most about 100 nM, for example, when measured in an ELISA assay essentially described in Example 5.

In some embodiments, the fusion polypeptides of disclosure are capable of inhibiting the binding of LAG-3 to MHC class II, such as those expressed on antigen-presenting cells (APCs) or tumor cells. The inhibitory mode of action can, for example, be determined by a FACS analysis as essentially described in Example 6.

In some embodiments, the fusion polypeptides of the disclosure may be able to induce IL-2 and/or IFN-γ production, reflective of T cell activation, in a functional T cell activation assay essentially described in Example 7 and 8 and may even demonstrate a tendency towards stronger IL-2 and/or IFN-γ induction at higher coating concentrations.

### A. Exemplary Immunoglobulins as Included in the Fusion Polypeptides.

In some embodiments, with respect to the fusion polypeptide, the first binding domain comprises a full-length immunoglobulin or an antigen-binding domain thereof specific for PD-1. The immunoglobulin, for example, may be IgG1, IgG2 or IgG4. In further embodiments, the immunoglobulin is a monoclonal antibody against PD-1.

Illustrative examples of PD-1-binding antibodies of the disclosure may comprises an antigen-binding region which cross-blocks or binds to the same epitope as a PD-1-binding antibody comprising the VH and VL regions of antibodies nivolumab (also known as ONO-4538, BMS-936558, or MDX1106, marketed as Opdivo), pembrolizumab (also referred to as lambrolizumab or MK03475, trade name Keytruda), PDR001, MEDIO0680 (formerly AMP-514), pidilizumab (CT-011), ENUM-388D4, or ENUM-244C8, all known in the art. In another particular embodiment, a PD-1-binding antibody of the disclosure may comprise an antigen-binding region, such as any one of the three heavy chain complementarity determining regions (CDRs) (HCDR1, HCDR2 and HCDR3) and the three light chain CDRs (LCDR1, LCDR2 and LCDR3), from an antibody selected from the group consisting of nivolumab, pembrolizumab, PDR001, MEDIO0680, pidilizumab, ENUM-388D4, and ENUM-244C8.

In some embodiments the antibody binding to PD-1 or antigen-binding domain thereof has an antigen-binding region which cross-blocks or binds to any one of the sequences selected from the group consisting of SEQ ID NOs: 124-154

In some embodiments the antibody binding to PD-1 will have the sequence of the benchmark antibody of SEQ ID NOs: 3 and 4 or the benchmark antibody of SEQ ID NOs: 47 and 48.

In some embodiments the PD-1 antibody or antigen-binding domain thereof will have a heavy chain variable region (HCVR) selected from the group consisting of SEQ ID NOs: 59-84, and a light chain variable region (LCVR) selected from the group consisting of SEQ ID NOs: 85-111. In other embodiments the PD-1 antibody or antigen-binding domain thereof will have a heavy chain variable region (HCVR) selected from the group consisting of SEQ ID NOs: 112-117 and a light chain variable region (LCVR) selected from the group consisting of SEQ ID NOs: 118-123.

In some embodiments the PD-1 antibody or antigen-binding domain will have a heavy chain comprising a HCVR that is any one of SEQ ID NOs: 59-84, 112-117 and a light chain comprising a LCVR that is any one of SEQ ID NOs: 85-111, 118-123.

In some embodiments the heavy chain and light chain pair of the PD-1 antibody comprise a HCVR and LCVR, respectively, as follows: SEQ ID NOs: 112 and 118; SEQ ID NOs: 112 and 119; SEQ ID NOs: 112 and 120; SEQ ID NOs: 112 and 121; SEQ ID NOs: 112 and 122; SEQ ID NOs: 112 and 123; SEQ ID NOs: 113 and 118; SEQ ID NOs: 113 and 119; SEQ ID NOs: 113 and 120; SEQ ID NOs: 113 and 121; SEQ ID NOs: 113 and 122; SEQ ID NOs: 113 and 123; SEQ ID NOs: 114 and 118; SEQ ID NOs: 114 and 119; SEQ ID NOs: 114 and 120; SEQ ID NOs: 114 and 121: SEQ ID NOs: 114 and 122; SEQ ID NOs: 114 and 123; SEQ ID NOs: 115 and 118; SEQ ID NOs: 115 and 119; SEQ ID NOs: 115 and 120; SEQ ID NOs: 115 and 121; SEQ ID NOs: 115 and 122; SEQ ID NOs: 115 and 123; SEQ ID NOs: 116 and 118; SEQ ID NOs: 116 and 119; SEQ ID NOs: 116 and 120; SEQ ID NOs: 116 and 121; SEQ ID NOs: 116 and 122; SEQ ID NOs: 116 and 123; SEQ ID NOs: 117 and 118; SEQ ID NOs: 117 and 119; SEQ ID NOs: 117 and 120; SEQ ID NOs: 117 and 121; SEQ ID NOs: 117 and 122; and SEQ ID NOs: 117 and 123.

In some embodiments the PD-1 antibody or antigen-binding domain thereof will have a heavy chain variable region (HCVR) with at least 70%, at least 80%, at least 85%, at least 90%, or at least 95% sequence identity to an amino acid sequence selected from the group consisting of SEQ ID NOs: 59-84, and a light chain variable region (LCVR) with at least 70%, at least 80%, at least 85%, at least 90%, or at least 95% sequence identity to an amino acid sequence selected from the group consisting of SEQ ID NOs: 85-111. In other embodiments the PD-1 antibody or antigen-binding domain thereof will have a heavy chain variable region (HCVR) with at least 70%, at least 80%, at least 85%, at least 90%, or at least 95% sequence identity to an amino acid sequence selected from the group consisting of SEQ ID NOs: 112-117 and a light chain variable region (LCVR) with at least 70%, at least 80%, at least 85%, at least 90%, or at least 95% sequence identity to an amino acid sequence selected from the group consisting of SEQ ID NOs: 118-123.

In some still preferred embodiments, an antibody of the disclosure specifically binding to PD-1 is nivolumab, pembrolizumab, PDR001, MEDIO0680, pidilizumab, ENUM-388D4, or ENUM-244C8 or the antigen-binding domain thereof.

Various patent applications disclose anti-PD-1 antibodies, production thereof, and/or methods of enhancing immune responses with such anti-PD-1 antibodies, including the following: U.S. Patent Application Publication Nos. US 2003/0039653, US 2004/0213795, US 2006/0110383, US 2007/0065427, US 2007/0122378, US 2009/0217401, US 2011/0008369, and US2015/0203579 and PCT International Application Publication Nos. WO 2003/099196, WO 2006/121168, WO 2007/005874, WO 2008/156712, WO 2009114335, WO 2010/027423, WO2 011/110604, WO 2012/145493, WO 2013/014668, WO 2014/194302, WO 2015/035606, and WO 2016/106159. The disclosure of each of these applications is hereby incorporated by reference in its entirety.

A PD-1-binding antibody of the disclosure may be any one of the anti-PD-1 antibodies disclosed in above mentioned applications.

A PD-1-binding antibody of the disclosure may comprise an antigen-binding region which cross-blocks or binds to the same epitope as a PD-1-binding antibody comprising the VH and VL regions of any one of the anti-PD-1 antibodies disclosed in above mentioned applications. In another particular embodiment, the PD-1-binding antibody may comprise an antigen-binding region, such as any one of the three heavy chain complementarity determining regions (CDRs) (HCDR1, HCDR2 and HCDR3) and the three light chain CDRs (LCDR1, LCDR2 and LCDR3), from any one of the anti-PD-1 antibodies disclosed in above mentioned applications.

In some embodiments the heavy chain variable region of the PD-1 antibody or antigen-binding domain thereof will have the three complementarity determining regions (CDRs) having following sequences: GYTFTDYE (HCDR1, SEQ ID NO: 163), IDPGTGGT (HCDR2, SEQ ID NO: 164), TSEKFGSNYYFDY (HCDR3; SEQ ID NO: 165). In some embodiments the heavy chain variable region of the PD-1 antibody or antigen-binding domain thereof will have the three complementarity determining regions (CDRs) having following sequences: GYTFTSYW (HCDR1, SEQ ID NO: 168), IDPSNSET (HCDR2, SEQ ID NO: 169), ARSRGNYAYEMDY (HCDR3; SEQ ID NO: 170). In some embodiments the heavy chain variable region of the PD-1 antibody or antigen-binding domain thereof will have the three complementarity determining regions (CDRs) having following sequences: GYTFTDYW (HCDR1, SEQ ID NO: 173), IDTSDSYT (HCDR2, SEQ ID NO: 174), ARRDYGGFGY (HCDR3; SEQ ID NO: 175). In some embodiments the heavy chain variable region of the PD-1 antibody or antigen-binding domain thereof will have the three complementarity determining regions (CDRs) having following sequences: GYTFTDYN (HCDR1, SEQ ID NO: 178), IDPNNGDT (HCDR2, SEQ ID NO: 179), ARWRSSMDY (HCDR3; SEQ ID NO: 180). In some embodiments the heavy chain variable region of the PD-1 antibody or antigen-binding domain thereof will have the three complementarity determining regions (CDRs) having following sequences: GYSITSDYA (HCDR1, SEQ ID NO: 183), ITYSGSP (HCDR2, SEQ ID NO: 184), ARGLGGHYFDY (HCDR3; SEQ ID NO: 185). In some embodiments the heavy chain variable region of the PD-1 antibody or antigen-binding domain thereof will have the three complementarity determining regions (CDRs) having following sequences: GFSLTSYG (HCDR1, SEQ ID NO: 188), IWRGGNT (HCDR2, SEQ ID NO: 189), AASMIGGY (HCDR3; SEQ ID NO: 190).

In some embodiments the light chain variable region of the PD-1 antibody or antigen-binding domain thereof will have the three complementarity determining regions (CDRs) having following sequences: QTIVHSDGNTY (LCDR1, SEQ ID NO: 166), KVS (LCDR2), FQGSHVPLT (LCDR3, SEQ ID NO: 167). In some embodiments the light chain variable region of the PD-1 antibody or antigen-binding domain thereof will have the three complementarity determining regions (CDRs) having following sequences: SSVSSNY (LCDR1, SEQ ID NO: 171), STS (LCDR2), HQWSSYPP (LCDR3, SEQ ID NO: 172). In some embodiments the light chain variable region of the PD-1 antibody or antigen-binding domain thereof will have the three complementarity determining regions (CDRs) having following sequences: QDISSY (LCDR1, SEQ ID NO: 176), YTS (LCDR2), QQYSELPW (LCDR3, SEQ ID NO: 177). In some embodiments the light chain variable region of the PD-1 antibody or antigen-binding domain thereof will have the three complementarity determining regions (CDRs) having following sequences: QGISNY (LCDR1, SEQ ID NO: 181), YTS (LCDR2), QQYSNLPW (LCDR3, SEQ ID NO: 182). In some embodiments the light chain variable region of the PD-1 antibody or antigen-binding domain thereof will have the three complementarity determining regions (CDRs) having following sequences: QSISDY (LCDR1, SEQ ID NO: 186), YAS (LCDR2), QNGRSYPY (LCDR3, SEQ ID NO: 187). In some embodiments the light chain variable region of the PD-1 antibody or antigen-binding domain thereof will have the three complementarity determining regions (CDRs) having following sequences: QSIVHSNGNTY (LCDR1, SEQ ID NO: 191), KVS (LCDR2), FQGSHVPL (LCDR3, SEQ ID NO: 192).

In some embodiments, the PD-1 antibody or antigen-binding domain thereof comprises a heavy chain variably region that will have the three complementarity determining regions (CDRs) having following sequences: GYTFTDYE (HCDR1, SEQ ID NO: 163), IDPGTGGT (HCDR2, SEQ ID NO: 164), TSEKFGSNYYFDY (HCDR3; SEQ ID NO: 165), and a light chain variably region that will have the three complementarity determining regions (CDRs) having following sequences: QTIVHSDGNTY (LCDR1, SEQ ID NO: 166), KVS (LCDR2), FQGSHVPLT (LCDR3, SEQ ID NO: 167). In some embodiments, the PD-1 antibody or antigen-binding domain thereof comprises a heavy chain variably region that will have the three complementarity determining regions (CDRs) having following sequences: GYTFTSYW (HCDR1, SEQ ID NO: 168), IDPSNSET (HCDR2, SEQ ID NO: 169), ARSRGNYAYEMDY (HCDR3; SEQ ID NO: 170), and a light chain variably region that will have the three complementarity determining regions (CDRs) having following sequences: SSVSSNY (LCDR1, SEQ ID NO: 171), STS (LCDR2), HQWSSYPP (LCDR3, SEQ ID NO: 172). In some embodiments, the PD-1 antibody or antigen-binding domain thereof comprises a heavy chain variably region that will have the three complementarity determining regions (CDRs) having following sequences: GYTFTDYW (HCDR1, SEQ ID NO: 173), IDTSDSYT (HCDR2, SEQ ID NO: 174), ARRDYGGFGY (HCDR3; SEQ ID NO: 175), and a light chain variably region that will have the three complementarity determining regions (CDRs) having following sequences: QDISSY (LCDR1, SEQ ID NO: 176), YTS (LCDR2), QQYSELPW (LCDR3, SEQ ID NO: 177). In some embodiments, the PD-1 antibody or antigen-binding domain thereof comprises a heavy chain variably region that will have the three complementarity determining regions (CDRs) having following sequences: GYTFTDYN (HCDR1, SEQ ID NO: 178), IDPNNGDT (HCDR2, SEQ ID NO: 179), ARWRSSMDY (HCDR3; SEQ ID NO: 180), and a light chain variably region that will have the three complementarity determining regions (CDRs) having following sequences: QGISNY (LCDR1, SEQ ID NO: 181), YTS (LCDR2), QQYSNLPW (LCDR3, SEQ ID NO: 182). In some embodiments, the PD-1 antibody or antigen-binding domain thereof comprises a heavy chain variably region that will have the three complementarity determining regions (CDRs) having following sequences: GYSITSDYA (HCDR1, SEQ ID NO: 183), ITYSGSP (HCDR2, SEQ ID NO: 184), ARGLGGHYFDY (HCDR3; SEQ ID NO: 185), and a light chain variably region that will have the three complementarity determining regions (CDRs) having following sequences: QSISDY (LCDR1, SEQ ID NO: 186), YAS (LCDR2), QNGRSYPY (LCDR3, SEQ ID NO: 187). In some embodiments, the PD-1 antibody or antigen-binding domain thereof comprises a heavy chain variably region that will have the three complementarity determining regions (CDRs) having following sequences: GFSLTSYG (HCDR1, SEQ ID NO: 188), IWRGGNT (HCDR2, SEQ ID NO: 189), AASMIGGY (HCDR3; SEQ ID NO: 190), and a light chain variably region that will have the three complementarity determining regions (CDRs) having following sequences: QSIVHSNGNTY (LCDR1, SEQ ID NO: 191), KVS (LCDR2), FQGSHVPL (LCDR3, SEQ ID NO: 192).

Unless otherwise indicated, all CDR sequences disclosed herein are defined according to the IMGT method as described in Lefranc, M.-P., The Immunologist, 7, 132-136 (1999). CDR1 consists of positions 27 to 38, CDR2 consists of positions 56 to 65, CDR3 for germline V-genes consists of positions 105 to 116, CDR3 for rearranged V-J-genes or V-D-J-genes consists of positions 105 to 117 (position preceding J-PHE or J-TRP 118) with gaps at the top of the loop for rearranged CDR3-IMGT with less than 13 amino acids, or with additional positions 112.1, 111.1, 112.2, 111.2, etc. for rearranged CDR3-IMGT with more than 13 amino acids. The positions given in this paragraph are according to the IMGT numbering described in Lefranc, M.-P., The Immunologist, 7, 132-136 (1999).

In some other embodiments, with respect to a PD-1-binding antibody of the disclosure, it is preferred that the antibody having silenced effector functions has mutations in F234 and L235, or, in positions D265 and P329, numbering according to EU index of Kabat (Johnson and Wu, Nucleic Acids Res, 2000).

The antibody specifically binding to PD-1 as included in the fusion polypeptides of the disclosure may comprise an Fc part which allows for extending the in vivo half-life of the bispecific binding molecule of the invention. Such Fc part is preferably from human origin, more preferably a human Fc part of an IgG1 or IgG4 antibody, even more preferably an engineered human Fc part of an IgG1 or IgG4 with activating or silencing effector functions, wherein silencing effector functions are preferred over activating effector functions. Most preferably, such an Fc part is an engineered to silence effector functions with a mutation at positions 234 and/or 235, numbering according to EU index of Kabat (Johnson and Wu, Nucleic Acids Res, 2000). In some embodiments, mutations in positions F234 and L235 of the anti-PD-1 antibody may be introduced to silence effector functions. In other embodiments, mutations in positions D265 and P329 of the anti-PD-1 antibody may be introduced, to silence effector function. Numbering for both sets of these potential mutations is according to the EU index of Kabat (Shields et al., J Biol Chem, 2001).

Various techniques for the production of antibodies and fragments thereof are well known in the art and described, e.g. in Altshuler et al. (Biochemistry (Mosc), 2010). Thus, polyclonal antibodies can be obtained from the blood of an animal following immunization with an antigen in mixture with additives and adjuvants and monoclonal antibodies can be produced by any technique which provides antibodies produced by continuous cell line cultures. Examples for such techniques are described, e.g. Harlow and Lane (1999), (1988), and include the hybridoma technique originally described by Köhler and Milstein, 1975, the trioma technique, the human B cell hybridoma technique (see e.g. Li et al., Proc Natl Acad Sci USA, 2006, Kozbor and Roder, Immunol Today, 1983) and the EBV-hybridoma technique to produce human monoclonal antibodies (Cole et al., Cancer Res, 1984). Furthermore, recombinant antibodies may be obtained from monoclonal antibodies or can be prepared de novo using various display methods such as phage, ribosomal, mRNA, or cell display. A suitable system for the expression of the recombinant (humanized) antibodies or fragments thereof may be selected from, for example, bacteria, yeast, insects, mammalian cell lines or transgenic animals or plants (see, e.g., U.S. Pat. No. 6,080,560; Holliger and Hudson, Nat Biotechnol, 2005). Further, techniques described for the production of single chain antibodies (see, inter alia, U.S. Pat. No. 4,946,778) can be adapted to produce single chain antibodies specific for the target of this invention. Surface plasmon resonance as employed in the BIAcore system can be used to increase the efficiency of phage antibodies.

### B. Exemplary LAG-3-Specific Lipocalin Muteins as Included in the Fusion Polypeptides.

As used herein, a “lipocalin” is defined as a monomeric protein of approximately 18-20 kDa in weight, having a cylindrical β-pleated sheet supersecondary structural region comprising a plurality of (preferably eight) β-strands connected pair-wise by a plurality of (preferably four) loops at one end to define thereby a binding pocket. It is the diversity of the loops in the otherwise rigid lipocalin scaffold that gives rise to a variety of different binding modes among the lipocalin family members, each capable of accommodating targets of different size, shape, and chemical character (reviewed, e.g. in Skerra, Biochim Biophys Acta, 2000, Flower et al., Biochim Biophys Acta, 2000, Flower, Biochem J, 1996). Indeed, the lipocalin family of proteins have naturally evolved to bind a wide spectrum of ligands, sharing unusually low levels of overall sequence conservation (often with sequence identities of less than 20%) yet retaining a highly conserved overall folding pattern. The correspondence between positions in various lipocalins is well known to one of skill in the art (see, e.g. U.S. Pat. No. 7,250,297).

As noted above, a lipocalin is a polypeptide defined by its supersecondary structure, namely cylindrical β-pleated sheet supersecondary structural region comprising eight β-strands connected pair-wise by four loops at one end to define thereby a binding pocket. The present disclosure is not limited to lipocalin muteins specifically disclosed herein. In this regard, the disclosure relates to lipocalin muteins having a cylindrical β-pleated sheet supersecondary structural region comprising eight β-strands connected pair-wise by four loops at one end to define thereby a binding pocket, wherein at least one amino acid of each of at least three of said four loops has been mutated as compared to the reference sequence, and wherein said lipocalin is effective to bind LAG-3 with detectable affinity.

In one particular embodiment, a lipocalin mutein disclosed herein is a mutein of human tear lipocalin (hTlc or TLPC), also termed lipocalin-1, human tear pre-albumin or von Ebner gland protein. The term “human tear lipocalin” or “hTlc” or “lipocalin-1” as used herein refers to the mature human tear lipocalin with the SWISS-PROT/UniProt Data Bank Accession Number P31025 (Isoform 1). The amino acid sequence shown in SwissProt/UniProt Data Bank Accession Number P31025 may be used as a preferred “reference sequence,” more preferably the amino acid sequence shown in SEQ ID NO: 1 is used herein as “reference sequence”.

In some embodiments, a lipocalin mutein binding LAG-3 with detectable affinity may include at least one amino acid substitution of a native cysteine residue of the reference sequence by another amino acid, for example, a serine residue. In some other embodiments, a lipocalin mutein binding LAG-3 with detectable affinity may include one or more non-native cysteine residues substituting one or more amino acids of a wild-type lipocalin. In a further particular embodiment, a lipocalin mutein according to the disclosure includes at least two amino acid substitutions of a native amino acid by a cysteine residue, hereby to form one or more cysteine bridges. In some embodiments, said cysteine bridge may connect at least two loop regions. The definition of these regions is used herein in accordance with Flower (Biochem J, 1996), (Biochim Biophys Acta, 2000) and Breustedt et al. (J Biol Chem, 2005). In a related embodiment, the disclosure teaches one or more lipocalin muteins that are capable of activating downstream signaling pathways of LAG-3 by binding to LAG-3.

Proteins of the disclosure, which are directed against or specific for LAG-3, include any number of specific-binding protein muteins that are based on a defined protein scaffold, preferably a lipocalin scaffold. Also preferably, the number of nucleotides or amino acids, respectively, that is exchanged, deleted or inserted is 1, 2, 3, 4, 5, 6, 7, 8, 9, 10, 11, 12, 13, 14, 15, 16, 17, 18, 19, 20 or more such as 25, 30, 35, 40, 45 or 50, with 1, 2, 3, 4, 5, 6, 7, 8, 9, 10, or 11 being preferred and 9, 10 or 11 being even more preferred. However, it is preferred that protein muteins of the disclosure is still capable of binding LAG-3.

In one aspect, the present disclosure includes various lipocalin muteins that bind LAG-3 with at least detectable affinity. In this sense, LAG-3 can be regarded as a non-natural ligand of the reference wild-type lipocalins, where “non-natural ligand” refers to a compound that does not bind to wild type lipocalin under physiological conditions. By engineering wild type lipocalins with one or more mutations at certain sequence positions, the present disclosure shows that high affinity and high specificity for the non-natural ligand, LAG-3, is possible. In some embodiments, at 1, 2, 3, 4, 5, 6, 7, 8, 9, 10, 11, 12, or even more nucleotide triplet(s) encoding certain sequence positions on wild type lipocalins, a random mutagenesis may be carried out through substitution at these positions by a subset of nucleotide triplets.

Further, the lipocalin muteins of the disclosure may have a mutated amino acid residue at any one or more, including at least at any 1, 2, 3, 4, 5, 6, 7, 8, 9, 10, 11 or 12, of the sequence positions corresponding to certain sequence positions of the linear polypeptide sequence of the reference lipocalin.

A lipocalin mutein of the disclosure may include the wild-type (natural) amino acid sequence of the “parental” protein scaffold (such as a lipocalin scaffold) outside the mutated amino acid sequence positions. In some embodiments, a lipocalin mutein according to the disclosure may also carry one or more amino acid mutations at one or more sequence position(s) as long as such a mutation does, at least essentially not hamper or not interfere with the binding activity and the folding of the mutein. Such mutations can be accomplished very easily on DNA level using established standard methods (Sambrook and Russell, 2001, Molecular cloning: a laboratory manual). Illustrative examples of alterations of the amino acid sequence are insertions or deletions as well as amino acid substitutions. Such substitutions may be conservative, i.e. an amino acid residue is replaced with an amino acid residue of chemically similar properties, in particular with regard to polarity as well as size. Examples of conservative substitutions are the replacements among the members of the following groups: 1) alanine, serine, and threonine; 2) aspartic acid and glutamic acid; 3) asparagine and glutamine; 4) arginine and lysine; 5) iso-leucine, leucine, methionine, and valine; and 6) phenylalanine, tyrosine, and tryptophan. On the other hand, it is also possible to introduce non-conservative alterations in the amino acid sequence. In addition, instead of replacing single amino acid residues, it is also possible to either insert or delete one or more continuous amino acids of the primary structure of the reference lipocalin, preferably hTlc, as long as these deletions or insertion result in a stable, folded and functional mutein. In such mutein, for instance, one or more amino acid residues are added or deleted at the N- or C-terminus of the polypeptide (for example, TIc muteins with truncated N- and C-terminus). Generally, such a mutein may have about at least 70%, including at least about 80%, such as at least about 85% amino acid sequence identity, with the amino acid sequence of hTlc (SEQ ID NO: 1). As an illustrative example, the present disclosure also encompasses TIc muteins as defined above, in which the first four N-terminal amino acid residues of the sequence of mature human tear lipocalin (His-His-Leu-Leu; positions 1-4) and/or the last two C-terminal amino acid residues (Ser-Asp; positions 157-158) of the linear polypeptide sequence of the mature human tear lipocalin have been deleted (SEQ ID NOs: 13-28).

The amino acid sequence of a lipocalin mutein disclosed herein has a high sequence identity to the reference lipocalin, preferably hTlc, when compared to sequence identities with other lipocalins. In this general context, the amino acid sequence of a lipocalin mutein of the disclosure is at least substantially similar to the amino acid sequence of the reference lipocalin, with the proviso that possibly there are gaps (as defined below) in an alignment that are the result of additions or deletions of amino acids. A respective sequence of a lipocalin mutein of the disclosure, being substantially similar to the sequences of the reference lipocalin, has, in some embodiments, at least 70% identity or sequence homology, at least 75% identity or sequence homology, at least 80% identity or sequence homology, at least 82% identity or sequence homology, at least 85% identity or sequence homology, at least 87% identity or sequence homology, or at least 90% identity or sequence homology including at least 95% identity or sequence homology, to the sequence of the reference lipocalin, with the proviso that the altered position or sequence is retained and that one or more gaps are possible.

As used herein, a lipocalin mutein of the disclosure “specifically binds” a target (for example, LAG-3) if it is able to discriminate between that target and one or more reference targets, since binding specificity is not an absolute, but a relative property. “Specific binding” can be determined, for example, in accordance with western blots, ELISA, FACS, RIA (radioimmunoassay), ECL (electrochemiluminescence), IRMA (immunoradiometric assay), IHC (ImmunoHistoChemistry), and peptide scans.

In one aspect, the present disclosure provides LAG-3-binding hTlc muteins.

In this regard, the disclosure provides one or more hTlc muteins that are capable of binding LAG-3 with an affinity measured by a K_{d} of about 300 nM or lower and even about 100 nM or lower.

In some embodiments, such hTlc mutein comprises mutated amino acid residue(s) at one or more positions corresponding to positions 14, 25-34, 36, 48, 52-53, 55-58, 60-61, 66, 79, 85-86, 101, 104-106, 108, 110-112, 114, 121, 140 and 153 of the linear polypeptide sequence of the hTlc (SEQ ID NO: 1).

In some particular embodiments, such hTlc muteins may contain mutated amino acid residue(s) at one or more positions corresponding to positions 26-34, 55-58, 60-61, 65, 104-106 and 108 of the linear polypeptide sequence of hTlc (SEQ ID NO: 1).

In further particular embodiments, such hTlc muteins may further include mutated amino acid residue(s) at one or more positions corresponding to positions 101, 111, 114 and 153 of the linear polypeptide sequence of hTlc (SEQ ID NO:1).

In other particular embodiments, the hTlc muteins may contain mutated amino acid residue(s) at one or more positions corresponding to positions 14, 25-34, 36, 48, 52-53, 55-58, 60-61, 66, 79, 85-86, 101, 104-106, 108, 110-112, 114, 121, 140 and 153 of the linear polypeptide sequence of the hTlc (SEQ ID NO: 1).

In some further embodiments, the hTlc muteins may comprise at least 1, 2, 3, 4, 5, 6, 7, 8, 9, 10, 11, 12, 13, 14, 15, 16, 17, 18, 19, 20, 21, 22, 23, 24, 25, 26 or even more, mutated amino acid residue(s) at one or more sequence positions corresponding to sequence positions 14, 25-34, 36, 48, 52-53, 55-58, 60-61, 66, 79, 85-86, 101, 104-106, 108, 110-112, 114, 121, 140 and 153 of the linear polypeptide sequence of the hTlc (SEQ ID NO: 1), and wherein said polypeptide binds LAG-3, in particular human LAG-3.

In some still further embodiments, the disclosure relates to a polypeptide, wherein said polypeptide is a hTlc mutein, in comparison with the linear polypeptide sequence of hTlc (SEQ ID NO: 1), comprising at least 1, 2, 3, 4, 5, 6, 7, 8, 9, 10, 11, 12 or even more, mutated amino acid residues at the sequence positions 14, 25-34, 36, 48, 52-53, 55-58, 60-61, 66, 79, 85-86, 101, 104-106, 108, 110-112, 114, 121, 140, and 153 and wherein said polypeptide binds LAG-3, in particular human LAG-3.

In some embodiments, a lipocalin mutein according to the disclosure may include at least one amino acid substitution of a native cysteine residue by e.g. a serine residue. In some embodiments, a hTlc mutein according to the disclosure includes an amino acid substitution of a native cysteine residue at positions 61 and/or 153 by another amino acid such as a serine residue. In this context it is noted that it has been found that removal of the structural disulfide bond (on the level of a respective naïve nucleic acid library) of wild-type tear lipocalin that is formed by the cysteine residues 61 and 153 (cf. Breustedt et al., J Biol Chem, 2005) may provide tear lipocalin muteins that are not only stably folded but are also able to bind a given non-natural ligand with high affinity. In some particular embodiments, the TIc mutein according to the disclosure includes the amino acid substitutions Cys 61→Ala, Phe, Lys, Arg, Thr, Asn, Gly, Gin, Asp, Asn, Leu, Tyr, Met, Ser, Pro or Trp, and/or Cys 153, Lys, Arg, Thr, A substitutions have proven useful to prevent the formation of the naturally occurring disulphide bridge linking Cys 61 and Cys 153, and thus to facilitate handling of the mutein. However, hTlc that binds LAG-3 and that have the disulphide bridge formed between Cys 61 and Cys 153 are also part of the present disclosure.

In some embodiments, the elimination of the structural disulfide bond may provide further advantage of allowing for the (spontaneous) generation or deliberate introduction of non-natural artificial disulfide bonds into muteins of the disclosure, thereby increasing the stability of the muteins. For example, in some embodiments, either two or all three of the cysteine codons at position 61, 101 and 153 are replaced by a codon of another amino acid. Further, in some embodiments, a hTlc mutein according to the disclosure includes an amino acid substitution of a native cysteine residue at position 101 by a serine residue or a histidine residue.

In some embodiments, a mutein according to the disclosure includes an amino acid substitution of a native amino acid by a cysteine residue at positions 28 or 105 with respect to the amino acid sequence of hTlc (SEQ ID NO: 1).

Further, in some embodiments, a mutein according to the disclosure includes an amino acid substitution of a native arginine residue at positions 111 by a proline residue with respect to the amino acid sequence of hTlc (SEQ ID NO: 1). Further, in some embodiments, a mutein according to the disclosure includes an amino acid substitution of a native lysine residue at positions 114 by a tryptophan residue or a glutamic acid with respect to the amino acid sequence of hTlc (SEQ ID NO: 1).

In some embodiments, a LAG-3-binding TIc mutein according to the disclosure includes, at one or more positions corresponding to positions 14, 25-34, 36, 48, 52-53, 55-58, 60-61, 66, 79, 85-86, 101, 104-106, 108, 110-112, 114, 121, 140, and 153 of the linear polypeptide sequence of the hTlc (SEQ ID NO: 1), one or more of the following mutated amino acid residues: Ser 14→Pro; Asp 25→Ser; Arg 26→Ser, Phe, Gly, Ala, Asp or Glu; Glu 27→Asp, Val or Thr; Phe 28→Cys or Asp; Pro 29→Phe, Leu or Trp; Glu 30→Trp, Asn or Tyr; Met 31→Ile, Val, Asp, Leu or Tyr; Asn 32→Asp, Glu, Tyr, Trp, Val, Thr or Met; Leu 33→Asp, Glu or Pro; Glu 34→Val, Trp or His; Val 36→Ala; Asn 48→Asp; Lys 52→Glu, Ser, Arg or Asn; Val 53→Ala; Met 55→Ala or Val; Leu 56→Asp, Gin or Asn; Ile 57→Leu; Ser 58→Phe, Trp or Asp; Arg 60→Phe or Glu; Cys 61→Trp, Pro, Leu or Trp; Ala 66→Asn; Ala 79→Glu; Val 85→Ala; Ala 86→Asp; Cys 101→Ser or Phe; Glu 104→Tyr; Leu 105→Cys or Gly; His 106→Ala, Glu, Thr, Tyr, Gin or Val; Lys 108→Tyr, Phe, Thr or Trp; Val 110→Gly or Ala; Arg 111→Pro; Gly 112→Met or Thr; Lys 114→Trp or Ala; Lys 121→Thr; Ser 140→Gly and Cys 153→Ser. In some embodiments, a hTlc mutein according to the disclosure includes two or more, such as 3, 4, 5, 6, 7, 8, 9, 10, 11, 12, even more such as 13, 14, 15, 16, 17, 18, 19, 20, 21, 22, 23, 24, 25, 26 or all mutated amino acid residues at these sequence positions of hTlc (SEQ ID NO:1).

In some additional embodiments, the LAG-3 binding hTlc muteins include one of the following sets of amino acid substitutions in comparison with the linear polypeptide sequence of the hTlc (SEQ ID NO:1).
(a) Arg 26→Ser; Glu 27→Asp; Phe 28→Cys; Pro 29→Phe; Glu 30→Trp; Met 31→Ile; Asn 32→Glu; Leu 33→Glu; Glu 34→Trp; Leu 56→Asp; Ser 58→Phe; Arg 60→Phe; Cys 61→Trp; Cys 101→Ser; Leu 105→Cys; His 106→Ala; Lys 108→Phe; Arg 111 Pro; Lys 114→Trp; Cys 153→Ser;
(b) Ser 14→Pro; Asp 25→Ser; Arg 26→Gly; Phe 28→Asp; Asn 32→Thr; Lys 52→Asn; Met 55→Ala; Ser 58→Asp; Ala 66→Asn; Ala 79→Glu; Ala 86→Asp; Cys 101→Phe; Leu 105→Gly; Lys 108→Thr; Val 110→Ala; Gly 112→Thr; Lys 114→Ala; Lys 121→Thr;
(c) Arg 26→Phe; Glu 27→Val; Phe 28→Cys; Pro 29→Leu; Glu 30→Tyr; Met 31→Asp; Asn 32→Val; Leu 33→Pro; Leu 56→Gin; Ser 58→Trp; Arg 60→Glu; Cys 61→Leu; Cys 101→Ser; Glu 104→Tyr; Leu 105→Cys; His 106→Val; Lys 108→Tyr; Arg 111→Pro; Lys 114→Trp; Cys 153→Ser;
(d) Arg 26→Glu; Glu 27→Thr; Phe 28→Cys; Pro 29→Trp; Glu 30→Trp; Met 31→Tyr; Asn 32→Val; Leu 33→Asp; Glu 34→His; Leu 56→Asn; Ile 57→Leu; Ser 58 Trp; Arg 60→Phe; Cys 61→Trp; Cys 101→Ser; Leu 105→Cys; His 106→Gin; Lys 108→Trp; Arg 111→Pro; Lys 114→Trp; Cys 153→Ser;
(e) Arg 26→Ser; Glu 27→Asp; Phe 28→Cys; Pro 29→Phe; Glu 30→Trp; Met 31→lie; Asn 32→Asp; Leu 33→Asp; Glu 34→Val; Leu 56→Asp; Ser 58→Phe; Arg 60→Phe; Cys 61→Trp; Cys 101→Ser; Leu 105→Cys; His 106→Ala; Lys 108→Tyr; Arg 111→Pro; Lys 114→Trp; Cys 153→Ser;
(f) Arg 26→Ser; Glu 27→Asp; Phe 28→Cys; Pro 29→Phe; Glu 30→Trp; Met 31→lie; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Ala; Lys 108→Tyr; Arg 111→Pro; Lys 114→Trp; Cys 153→Ser;
(g) Arg 26→Ser; Glu 27→Asp; Phe 28→Cys; Pro 29→Phe; Glu 30→Trp; Met 31→lie; Asn 32→Glu; Leu 33→Glu; Glu 34→Trp; Val 36→Ala; Asn 48→Asp; Leu 56→Asp; Ser 58→Phe; Arg 60→Phe; Cys 61→Trp; Val 85→Ala; Cys 101→Ser; Leu 105→Cys; His 106→Ala; Lys 108→Phe; Arg 111→Pro; Lys 114→Trp; Ser 140→Gly; Cys 153→Ser;
(h) Arg 26→Ser; Glu 27→Asp; Phe 28→Cys; Pro 29→Phe; Glu 30→Trp; Met 31→lie; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Glu; Lys 108→Phe; Arg 111→Pro; Lys 114→Trp; Cys 153→Ser;
(i) Arg 26→Ser; Glu 27→Asp; Phe 28→Cys; Pro 29→Phe; Glu 30→Trp; Met 31→lie; Asn 32→Glu; Leu 33→Glu; Glu 34→Trp; Val 36→Ala; Lys 52→Glu; Val 53→Ala; Leu 56→Asp; Ser 58→Phe; Arg 60→Phe; Cys 61→Trp; Cys 101→Ser; Leu 105→Cys; His 106→Ala; Lys 108→Phe; Arg 111→Pro; Lys 114→Trp; Cys 153→Ser;
(j) Arg 26→Ser; Glu 27→Asp; Phe 28→Cys; Pro 29→Phe; Glu 30→Trp; Met 31→Val; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Ala; Lys 108→Phe; Arg 111 Pro; Lys 114→Trp; Cys 153→Ser;
(k) Ser 14→Pro; Asp 25→Ser; Arg 26→Gly; Phe 28→Asp; Met 31→Leu; Asn 32→Trp; Lys 52→Ser; Met 55→Ala; Ser 58→Asp; Ala 66→Asn; Ala 79→Glu; Ala 86 Asp; Cys 101→Phe; Leu 105→Gly; His 106→Tyr; Lys 108→Thr; Val 110 Gly; Gly 112→Met; Lys 114→Ala; Lys 121→Thr;
(l) Ser 14→Pro; Asp 25→Ser; Arg 26→Ala; Phe 28→Asp; Met 31→Leu; Asn 32 Val; Lys 52→Ser; Met 55→Ala; Ser 58→Asp; Ala 66→Asn; Ala 79→Glu; Ala 86 Asp; Cys 101→Phe; Leu 105→Gly; Lys 108→Thr; Val 110→Ala; Gly 112 Thr; Lys 114→Ala; Lys 121→Thr;
(m) Ser 14→Pro; Asp 25→Ser; Arg 26→Asp; Phe 28→Asp; Asn 32→Thr; Lys 52→Ser; Met 55→Ala; Ser 58→Asp; Ala 66→Asn; Ala 79→Glu; Ala 86→Asp; Cys 101→Phe; Leu 105→Gly; His 106→Gin; Lys 108→Thr; Val 110→Gly; Gly 112→Met; Lys 114→Ala; Lys 121→Thr;
(n) Ser 14→Pro; Asp 25→Ser; Arg 26→Glu; Phe 28→Asp; Asn 32→Thr; Lys 52→Ser; Met 55→Ala; Ser 58→Asp; Ala 66→Asn; Ala 79→Glu; Ala 86→Asp; Cys 101→Phe; Leu 105→Gly; Lys 108→Thr; Val 110→Gly; Gly 112→Met; Lys 114→Ala; Lys 121→Thr;
(o) Ser 14→Pro; Asp 25→Ser; Arg 26→Gly; Phe 28→Asp; Asn 32→Met; Lys 52→Arg; Met 55→Val; Ser 58→Asp; Ala 66→Asn; Ala 79→Glu; Ala 86→Asp; Cys 101→Phe; Leu 105→Gly; His 106→Gin; Lys 108→Thr; Val 110→Gly; Gly 112→Met; Lys 114→Ala; Lys 121→Thr; or
(p) Arg 26→Phe; Glu 27→Val; Phe 28→Cys; Pro 29→Leu; Glu 30→Asn; Met 31→Asp; Asn 32→Tyr; Leu 33→Pro; Leu 56→Gin; Ser 58→Trp; Arg 60→Glu; Cys 61→Pro; Cys 101→Ser; Glu 104→Tyr; Leu 105→Cys; His 106→Thr; Lys 108→Tyr; Arg 111→Pro; Lys 114→Trp; Cys 153→Ser.

In the residual region, i.e. the region differing from sequence positions 14, 25-34, 36, 48, 52-53, 55-58, 60-61, 66, 79, 85-86, 101, 104-106, 108, 110-112, 114, 121, 140 and 153, a hTlc mutein of the disclosure may include the wild-type (natural) amino acid sequence outside the mutated amino acid sequence positions.

In still further embodiments, a hTlc mutein according to the current disclosure has at least 70% sequence identity or at least 70% sequence homology to the sequence of hTlc (SEQ ID NO: 1). As an illustrative example, the mutein of the SEQ ID NO: 20 has an amino acid sequence identity or a sequence homology of approximately 86% with the amino acid sequence of hTlc (SEQ ID NO:1).

In further particular embodiments, a hTlc mutein of the disclosure comprises an amino acid sequence as set forth in any one of SEQ ID NOs: 13-28 or a fragment or variant thereof.

In further particular embodiments, a hTlc mutein of the disclosure has at least 75%, at least 80%, at least 85% or higher sequence identity to an amino acid sequence selected from the group consisting of SEQ ID NOs: 13-28.

The disclosure also includes structural homologues of a hTlc mutein having an amino acid sequence selected from the group consisting of SEQ ID NOs: 13-28, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hTlc mutein.

A hTlc mutein according to the present disclosure can be obtained by means of mutagenesis of a naturally occurring form of hTlc (SEQ ID NO:1). In some embodiments of the mutagenesis, a substitution (or replacement) is a conservative substitution. Nevertheless, any substitution—including non-conservative substitution or one or more from the exemplary substitutions below—is envisaged as long as the lipocalin mutein retains its capability to bind to LAG-3, and/or it has a sequence identity to the then substituted sequence in that it is at least 60%, such as at least 65%, at least 70%, at least 75%, at least 80%, at least 85% or higher sequence identity to the amino acid sequence of the hTlc (SEQ ID NO:1.

In some particular embodiments, the present disclosure provides a lipocalin mutein that binds human LAG-3 with an affinity measured by a K_{d} of about 15 nM or lower, wherein the lipocalin mutein has at least 90% or higher, such as 95%, sequence identity to the amino acid sequence of any one of SEQ ID NO: 17 and SEQ ID NO: 27.

In one embodiment, the lipocalin muteins of the disclosure are fused at its N-terminus and/or its C-terminus to a fusion partner which is a protein domain that extends the serum half-life of the mutein. In further particular embodiments, the protein domain is an Fc part of an immunoglobulin, a C_{H}3 domain of an immunoglobulin, a C_{H} domain of an immunoglobulin, an albumin binding peptide or an albumin binding protein.

In another embodiment, the lipocalin muteins of the disclosure are conjugated to a compound that extends the serum half-life of the mutein. More preferably, the muteins are conjugated to a compound selected from the group consisting of a polyalkylene glycol molecule, a hydroethylstarch, an Fc part of an immunoglobulin, a C_{H}3 domain of an immunoglobulin, a C_{H}4 domain of an immunoglobulin, an albumin binding peptide, and an albumin binding protein.

In yet another embodiment, the current disclosure relates to a nucleic acid molecule comprising a nucleotide sequence encoding a lipocalin mutein disclosed herein. The disclosure encompasses a host cell containing said nucleic acid molecule.

### C. EXEMPLARY USES, APPLICATIONS AND PRODUCTION OF FUSION POLYPEPTIDES SPECIFIC for LAG-3 and PD-1.

It has been reported that LAG-3 plays an important role in promoting regulatory T cell (Treg) activity and in negatively regulating T cell activation and proliferation (Workman and Vignali, J Immunol, 2005). Both natural and induced Treg express elevated level of LAG-3, which is required for their maximal suppressive function (Huang et al., Immunity, 2004, Camisaschi et al., J Immunol, 2010). Furthermore, ectopic expression of LAG-3 on CD4+ effector T cells reduces their proliferative capacity and confers on their regulatory potential against third party T cells (Huang et al., Immunity, 2004). Recent studies have also shown that high LAG-3 expression on exhausted lymphocytic choriomeningitis virus (LCMV)-specific CD8+ T cells contributes to their unresponsive state and limits CD8+ T cell antitumor responses (Grosso et al., J Clin Invest, 2007, Blackburn et al., Nat Immunol, 2009). In fact, LAG-3 maintained tolerance to self and tumor antigens via direct effects on CD8+ T cells in 2 murine models (Grosso et al., J Clin Invest, 2007).

Immune tolerance observed in the setting of tumor development and tumor recurrence, however, seems to be mediated by the co-expression of various T cell negative regulatory receptors, not solely by LAG-3. Data from chronic viral infection models (Grosso et al., J Clin Invest, 2007, Blackburn et al., Nat Immunol, 2009, Lyford-Pike et al., Cancer Res, 2013), knock-out mice (Woo et al., Cancer Res, 2012, Bettini et al., J Immunol, 2011, Okazaki et al., J Exp Med, 2011), tumor recurrence models (Goding et al., J Immunol, 2013) and, to a more limited extent, human cancer patients (Goding et al., J Immunol, 2013, Gandhi et al., Blood, 2006, Matsuzaki et al., Proc Natl Acad Sci USA, 2010) support a model wherein T cells that are continuously exposed to antigen become progressively inactivated through a process termed “exhaustion”. Exhausted T cells are characterized by the expression of T cell negative regulatory receptors, predominantly PD-1, and LAG-3, whose action is to limit the cell's ability to proliferate, produce cytokines, and kill target cells and/or to increase Treg activity. However, the timing and sequence of expression of these molecules in the development and recurrence of tumors have not been fully characterized.

PD-1 is a cell surface signaling receptor that plays a critical role in the regulation of T cell activation and tolerance (Keir et al., Annu Rev Immunol, 2008). It is a type I transmembrane protein and together with BTLA, CTLA-4, ICOS and CD28, comprise the CD28 family of T cell co-stimulatory receptors. PD-1 is primarily expressed on activated T cells, B cells, and myeloid cells (Dong et al., Nat Med, 1999). It is also expressed on natural killer (NK) cells (Terme et al., Cancer Res, 2011).

Binding of PD-1 by its ligands, PD-L1 and PD-L2, results in phosphorylation of the tyrosine residue in the proximal intracellular immune receptor tyrosine inhibitory domain, followed by recruitment of the phosphatase SHP-2, eventually resulting in down-regulation of T cell activation. One important role of PD-1 is to limit the activity of T cells in peripheral tissues at the time of an inflammatory response to infection, thus limiting the development of autoimmunity (Pardoll, Nat Rev Cancer, 2012). Evidence of this negative regulatory role comes from the finding that PD-1-deficient mice develop lupus-like autoimmune diseases including arthritis and nephritis, along with cardiomyopathy (Nishimura et al., Science, 2001, Nishimura et al., Immunity, 1999). In the tumor setting, the consequence is the development of immune resistance within the tumor microenvironment. PD-1 is highly expressed on tumor-infiltrating lymphocytes, and its ligands are up-regulated on the cell surface of many different tumors (Dong et al., Nat Med, 2002). Multiple murine cancer models have demonstrated that binding of ligand to PD-1 results in immune evasion. In addition, blockade of this interaction results in anti-tumor activity (Hamid et al., N Engl J Med, 2013, Topalian et al., N Engl J Med, 2012).

There is a strong synergy between the PD-1 and LAG-3 inhibitory pathways in tolerance to both self and tumor antigens, therefore, dual blockade of the targets represents a promising combinatorial strategy for cancer (Woo et al., Cancer Res, 2012).

By simultaneously targeting immune checkpoints PD-1 and LAG-3, the fusion polypeptide of the disclosure may generate a durable anti-tumor and/or anti-infection response, increase anti-tumor lymphocyte cell activity, and enhance anti-tumor immunity, thereby produce synergistic anti-tumor results.

Numerous possible applications for the fusion polypeptides of the disclosure, therefore, exist in medicine. In some embodiments, fusion polypeptides of the disclosure may produce synergistic effect through dual-targeting of PD-1 and LAG-3.

In one aspect, the disclosure relates to the use of the fusion polypeptides disclosed herein for detecting PD-1 and LAG-3 in a sample as well as a respective method of diagnosis.

In another aspect, the disclosure features the use of one or more fusion polypeptides disclosed herein or of one or more compositions comprising such polypeptides for simultaneously binding of PD-1 and LAG-3.

The present disclosure also involves the use of one or more fusion polypeptides as described for complex formation with PD-1 and LAG-3.

Therefore, in a still further aspect of the disclosure, the disclosed one or more fusion polypeptides are used for the detection of PD-1 and LAG-3. Such use may include the steps of contacting one or more said fusion polypeptides, under suitable conditions, with a sample suspected of containing PD-1 and LAG-3, thereby allowing formation of a complex between the fusion polypeptides and PD-1 and LAG-3, and detecting the complex by a suitable signal. The detectable signal can be caused by a label, as explained above, or by a change of physical properties due to the binding, i.e. the complex formation, itself. One example is surface plasmon resonance, the value of which is changed during binding of binding partners from which one is immobilized on a surface such as a gold foil.

The fusion polypeptides disclosed herein may also be used for the separation of PD-1 and LAG-3. Such use may include the steps of contacting one or more said fusion polypeptides, under suitable conditions, with a sample supposed to contain PD-1 and LAG-3, thereby allowing formation of a complex between the fusion polypeptides and PD-1 and LAG-3, and separating the complex from the sample.

In still another aspect, the present disclosure features a diagnostic or analytical kit comprising a fusion polypeptide according to the disclosure.

In addition to their use in diagnostics, in yet another aspect, the disclosure contemplates a pharmaceutical composition comprising a fusion polypeptide of the disclosure and a pharmaceutically acceptable excipient.

Furthermore, the present disclosure provides fusion polypeptides that simultaneously bind PD-1 and LAG-3 for use as anti-infection and/or anti-cancer agents, and immune modulators. The fusion polypeptides of the present disclosure are envisaged to be used in a method of treatment or prevention of human diseases, such as a variety of tumors and autoinflammation in a subject in need thereof, comprising administering to said subject a therapeutically effective amount of one or more fusion polypeptides of the disclosure.

Examples of cancers that may be treated using the fusion polypeptides of the disclosure, include liver cancer, bone cancer, pancreatic cancer, skin cancer, head and neck cancer, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular 20 cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of 25 childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies 30 including, for example, multiple myeloma, B cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T cell lymphoma, and precursor T-lymphoblastic lymphoma, and any combinations of said cancers. The present invention is also applicable to treatment of metastatic cancers.

In one embodiment, the human patient suffers from non-small cell lung cancer (NSCLC) or a virally-related cancer (e.g., a human papilloma virus (HPV)-related tumor) or gastric adenocarcinoma. In a particular embodiment, the HPV-related tumor is HPV+ head and neck cancer (HNC). In another particular embodiment, the gastric adenocarcinoma is associated with Epstein-Barr virus (EBV) infection.

In another embodiment, the present disclosure also relates to nucleic acid molecules (DNA and RNA) that include nucleotide sequences encoding the fusion polypeptides disclosed herein. In yet another embodiment, the disclosure encompasses a host cell containing said nucleic acid molecule. Since the degeneracy of the genetic code permits substitutions of certain codons by other codons specifying the same amino acid, the disclosure is not limited to a specific nucleic acid molecule encoding a fusion polypeptide as described herein but encompasses all nucleic acid molecules that include nucleotide sequences encoding a functional polypeptide. In this regard, the present disclosure also relates to nucleotide sequences encoding the fusion polypeptides of the disclosure.

In some embodiments, a nucleic acid molecule encoding a lipocalin mutein disclosed in this application, such as DNA, may be “operably linked” to another nucleic acid molecule encoding an immunoglobulin of the disclosure to allow expression of a fusion polypeptide disclosed herein. In this regard, an operable linkage is a linkage in which the sequence elements of one nucleic acid molecule and the sequence elements of another nucleic acid molecule are connected in a way that enables expression of the fusion polypeptide as a single polypeptide.

The disclosure also relates to a method for the production the fusion polypeptides of the disclosure starting from the nucleic acid coding for the polypeptides or any subunits therein by means of genetic engineering methods. In some embodiments, the method can be carried out in vivo, wherein the fusion polypeptide can, for example, be produced in a bacterial or eukaryotic host organism, and then isolated from this host organism or its culture. It is also possible to produce a fusion polypeptide of the disclosure in vitro, for example, by using an in vitro translation system.

When producing the fusion polypeptide in vivo, a nucleic acid encoding such polypeptide is introduced into a suitable bacterial or eukaryotic host organism by means of recombinant DNA technology (as already outlined above). For this purpose, the host cell is first transformed with a cloning vector that includes a nucleic acid molecule encoding a fusion polypeptide as described herein using established standard methods. The host cell is then cultured under conditions, which allow expression of the heterologous DNA and thus the synthesis of the corresponding polypeptide. Subsequently, the polypeptide is recovered either from the cell or from the cultivation medium.

In one embodiment of the disclosure, the method includes subjecting at least one nucleic acid molecule encoding fusion polypeptides to mutagenesis at nucleotide triplets coding for at least one, sometimes even more, of the sequence positions corresponding to the sequence positions 14, 25-34, 36, 48, 52-53, 55-58, 60-61, 66, 79, 85-86, 101, 104-106, 108, 110-112, 114, 121, 140 and 153 of the linear polypeptide sequence of hTlc (SEQ ID NO: 1), as included in the fusion polypeptides.

In addition, with respect to hTlc muteins of the disclosure as included in the fusion polypeptides, the naturally occurring disulfide bond between Cys 61 and Cys 153 may be removed. Accordingly, such muteins can be produced in a cell compartment having a reducing redox milieu, for example, in the cytoplasm of Gram-negative bacteria.

The disclosure also includes nucleic acid molecules encoding the lipocalin muteins of the disclosure, which include additional mutations outside the indicated sequence positions of experimental mutagenesis. Such mutations are often tolerated or can even prove to be advantageous, for example if they contribute to an improved folding efficiency, serum stability, thermal stability or ligand binding affinity of the lipocalin muteins.

A nucleic acid molecule disclosed in this application may be “operably linked” to one or more regulatory sequence(s) to allow expression of this nucleic acid molecule.

A nucleic acid molecule, such as DNA, is referred to as “capable of expressing a nucleic acid molecule” or “able to allow expression of a nucleotide sequence” if it includes sequence elements that contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions include a promoter, which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/-10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the disclosure can include a regulatory sequence, such as a promoter sequence. In some embodiments a nucleic acid molecule of the disclosure includes a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

The nucleic acid molecules of the disclosure can also be part of a vector or any other kind of cloning vehicle, such as a plasmid, a phagemid, a phage, a baculovirus, a cosmid or an artificial chromosome.

In one embodiment, the nucleic acid molecule is included in a phasmid. A phasmid vector denotes a vector encoding the intergenic region of a temperate phage, such as M13 or f1, or a functional part thereof fused to the cDNA of interest. After superinfection of the bacterial host cells with such an phagemid vector and an appropriate helper phage (e.g. M13K07, VCS-M13 or R408) intact phage particles are produced, thereby enabling physical coupling of the encoded heterologous cDNA to its corresponding polypeptide displayed on the phage surface (Lowman, Annu Rev Biophys Biomol Struct, 1997, Rodi and Makowski, Curr Opin Biotechnol, 1999).

Such cloning vehicles can include, aside from the regulatory sequences described above and a nucleic acid sequence encoding a fusion polypeptide as described herein, replication and control sequences derived from a species compatible with the host cell that is used for expression as well as selection markers conferring a selectable phenotype on transformed or transfected cells. Large numbers of suitable cloning vectors are known in the art, and are commercially available.

The DNA molecule encoding a fusion polypeptide as described herein (for example, SEQ ID NOs: 29-36), and in particular a cloning vector containing the coding sequence of such a polypeptide can be transformed into a host cell capable of expressing the gene. Transformation can be performed using standard techniques. Thus, the disclosure is also directed to a host cell containing a nucleic acid molecule as disclosed herein.

The transformed host cells are cultured under conditions suitable for expression of the nucleotide sequence encoding a fusion polypeptide of the disclosure. Suitable host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g., HeLa cells or CHO cells) or primary mammalian cells.

In some embodiments where a lipocalin mutein of the disclosure, including as comprised in a fusion polypeptide disclosed herein, includes intramolecular disulphide bonds, it may be preferred to direct the nascent polypeptide to a cell compartment having an oxidizing redox milieu using an appropriate signal sequence. Such an oxidizing environment may be provided by the periplasm of Gram-negative bacteria such as E. coli, in the extracellular milieu of Gram-positive bacteria or in the lumen of the endoplasmic reticulum of eukaryotic cells and usually favors the formation of structural disulphide bonds.

In some embodiments, it is also possible to produce a fusion polypeptide of the disclosure in the cytosol of a host cell, preferably E. coli. In this case, the polypeptide can either be directly obtained in a soluble and folded state or recovered in form of inclusion bodies, followed by renaturation in vitro. A further option is the use of specific host strains having an oxidizing intracellular milieu, which may thus allow the formation of disulfide bonds in the cytosol (Venturi et al., J Mol Biol, 2002).

In some embodiments, a fusion polypeptide of the disclosure as described herein may be not necessarily generated or produced only by use of genetic engineering. Rather, such polypeptide can also be obtained by chemical synthesis such as Merrifield solid phase polypeptide synthesis or by in vitro transcription and translation. It is, for example, possible that promising fusion polypeptides and/or lipocalin muteins included in such fusion polypeptides, are identified using molecular modeling, synthesized in vitro, and investigated for the binding activity for target(s) of interest. Methods for the solid phase and/or solution phase synthesis of proteins are well known in the art (see e.g. Bruckdorfer et al., Curr Pharm Biotechnol, 2004).

In another embodiment, a fusion polypeptide of the disclosure may be produced by in vitro transcription/translation employing well-established methods known to those skilled in the art.

The skilled worker will appreciate methods useful to prepare fusion polypeptides contemplated by the present disclosure but whose protein or nucleic acid sequences are not explicitly disclosed herein. As an overview, such modifications of the amino acid sequence include, e.g., directed mutagenesis of single amino acid positions in order to simplify sub-cloning of a polypeptide gene or its parts by incorporating cleavage sites for certain restriction enzymes. In addition, these mutations can also be incorporated to further improve the affinity of a fusion polypeptide for its targets (e.g. PD-1 and LAG-3). Furthermore, mutations can be introduced to modulate certain characteristics of the polypeptide such as to improve folding stability, serum stability, protein resistance or water solubility or to reduce aggregation tendency, if necessary. For example, naturally occurring cysteine residues may be mutated to other amino acids to prevent disulphide bridge formation.

The fusion polypeptides of the disclosure may be prepared by any of the many conventional and well known techniques such as plain organic synthetic strategies, solid phase-assisted synthesis techniques or by commercially available automated synthesizers. On the other hand, they may also be prepared by conventional recombinant techniques alone or in combination with conventional synthetic techniques. A fusion polypeptide according to the present disclosure may be obtained by combining compounds as defined in chapters (A) and (B) herein above.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art, and that such modifications and variations are considered to be within the scope of this disclosure.

### V. EXAMPLES

### Example 1: Expression and Analysis of Representative Fusion Polypeptides

To engage PD-1 and LAG-3 at the same time, we generated several representative antibody-lipocalin mutein fusion polypeptides, fusing together the PD-1 specific antibody having the heavy and light chains provided by SEQ ID NO: 3 and SEQ ID NO: 4, respectively, and the LAG-3 specific lipocalin muteins of SEQ ID NO: 17 or SEQ ID NO: 27 via an unstructured (G₄S)₃ linker (SEQ ID NO: 2). The different formats that were generated are depicted in FIG. 1 a-e. Such fusion polypeptides (SEQ ID NOs: 5 and 4; SEQ ID NOs: 9 and 4, SEQ ID NOs: 6 and 4, SEQ ID NOs: 10 and 4; SEQ ID NOs: 3 and 7; SEQ ID NOs: 3 and 11, SEQ ID NOs: 3 and 8, and SEQ ID NOs: 3 and 12, respectively) were generated via fusion of either the lipocalin mutein of SEQ ID NO: 17 or the lipocalin mutein of SEQ ID NO: 27 to either one of the four termini of the antibody comprising of the heavy chain of SEQ ID NO: 3 and the light chain of SEQ ID NO: 4. When lipocalin muteins were fused to the N-terminus of either the heavy or the light chain of the antibody, they contained two additional amino acids, serine and aspartate, at the C-terminus before the linker sequence (SEQ ID NO: 2). The PD-1 specific antibody comprising of the heavy chain of SEQ ID NO: 3 and the light chain of SEQ ID NO: 4 had an engineered IgG4 backbone, which contained a S228P mutation to minimize IgG4 half-antibody exchange in-vitro and in-vivo (Silva et al., J Biol Chem, 2015). In addition, lipocalin mutein Fc fusions were generated by fusing the LAG-3 specific lipocalin muteins of SEQ ID NO: 17 or SEQ ID NO: 27 via an unstructured (G₄S)₃ linker (SEQ ID NO: 2) to the C-terminus of the Fc part of SEQ ID NO: 3. The two different constructs are depicted in FIG. 1 (SEQ ID NO: 41 and SEQ ID NO: 42). FIG. 1 f-i additionally shows the design of additional fusion polypeptides and corresponding sequences for such polypeptides where made based on an antibody specific for PD-1 (e.g. the antibody of SEQ ID NOs: 3 and 4 or the antibody of SEQ ID NOs: 47 and 48) and one or more lipocalin muteins specific for LAG-3 (e.g. the lipocalin mutein of SEQ ID NO: 17 or the lipocalin mutein of SEQ ID NO: 27).

The constructs of the fusion polypeptides were generated by gene synthesis and cloned into a mammalian expression vector. They were then transiently expressed in Expi293F™ cells (Life Technologies). The concentration of fusion polypeptides in the cell culture medium was measured either with a ForteBio Protein A sensor (Pall Corp.) or by HPLC (Agilent Technologies) employing a POROS® protein A affinity column (Applied Biosystems).

Likewise, to engage PD-1 and LAG-3 at the same time, the PD-1 specific antibody having the heavy and light chains provided by SEQ ID NO: 47 and SEQ ID NO: 48, respectively, and the LAG-3 specific lipocalin muteins of SEQ ID NO: 17 or SEQ ID NO: 27 can be fused together, e.g. via an unstructured (G₄S)₃ linker (SEQ ID NO: 2). Different formats can be generated; see FIG. 1, mutatis mutandis. Such different formats can be generated in analogy, as described above for PD-1-LAG-3 antibody-lipocalin mutein fusion polypeptides, fusing together the PD-1 specific antibody having the heavy and light chains provided by SEQ ID NO: 3 and SEQ ID NO: 4, respectively, and the LAG-3 specific lipocalin muteins of SEQ ID NO: 17 or SEQ ID NO: 27, with the exception that as the heavy and light chains the amino acid sequence of SEQ ID NO: 47 and SEQ ID NO: 48 are used.

Specifically, FIG. 1 shows additional representative fusion polypeptides that may be made by the same methods described herein using a different antibody specific for PD-1 (e.g. the antibody of SEQ ID NOs: 47 and 48) and one or more lipocalin muteins specific for LAG-3 (e.g. the lipocalin mutein of SEQ ID NO: 17 or the lipocalin mutein of SEQ ID NO: 27). The lipocalin muteins may be genetically fused to either the C- or the N-terminus of either the heavy chain or the light chain of the PD-1 specific antibody as depicted in FIG. 1 to yield the fusion polypeptides of SEQ ID NOs: 51 and 48, SEQ ID NOs: 55 and 48, SEQ ID NOs: 52 and 48, SEQ ID NOs: 56 and 48, SEQ ID NOs: 47 and 53, SEQ ID NOs: 47 and 57, SEQ ID NOs: 47 and 54, and SEQ ID NOs: 47 and 58.

The fusion polypeptides were purified using Protein A chromatography followed by size-exclusion chromatography (SEC) in phosphate-buffered saline (PBS). After SEC purification, the fractions containing monomeric protein are pooled and analyzed again using analytical SEC. The titers of the constructs after Protein A purification and extrapolated to 1 liter were as described in Table 1 below. Expression of the fusion polypeptides is in the same range as for the antibody.

TABLE 1
Expression titers for transient expression in Expi293F ™ cells.
Extrapolated to a 1 I expression scale.

| | Expression titer |
|---|---|
| Clone Name | [mg/L] |
| SEQ ID NOs: 5 and 4 | 175 |
| SEQ ID NOs: 6 and 4 | 154 |
| SEQ ID NOs: 3 and 7 | 209 |
| SEQ ID NOs: 3 and 8 | 127 |
| SEQ ID NOs: 9 and 4 | 127 |
| SEQ ID NOs: 10 and 4 | 110 |
| SEQ ID NOs: 3 and 11 | 161 |
| SEQ ID NOs: 3 and 12 | 152 |
| SEQ ID NO: 41 | 375 |
| SEQ ID NO: 42 | 345 |

### Example 2: Binding of Fusion Polypeptides Towards PD-1 in Enzyme-Linked Immunosorbent Assay (ELISA)

We employed an enzyme-linked immunosorbent assay (ELISA) assay to determine the binding potency of the fusion polypeptides to recombinant human PD-1-His (PD-1 with a C-terminal polyhistidine tag, ACROBiosystems). PD-1-His at the concentration of 1 μg/mL in PBS was coated overnight on microtiter plates at 4° C. After washing with PBS-0.05% T (PBS supplemented with 0.05% (v/v) Tween 20), the plates were blocked with 2% BSA (w/v) in PBS-0.1% T (PBS supplemented with 0.1% (v/v) Tween 20) for 1 h at room temperature. After washing with 100 μL PBS-0.05% T five times, the benchmark antibody (SEQ ID NOs: 3 and 4) or the fusion polypeptides at different concentrations were added to the wells and incubated for 1 h at room temperature, followed by another wash step. Bound antibodies/fusion polypeptides under study were detected after incubation with 1:5000 diluted anti-human IgG Fc-HRP (Jackson Laboratory) in PBS-0.1% T-2% BSA. After an additional wash step, fluorogenic HRP substrate (QuantaBlu, Thermo) was added to each well and the fluorescence intensity was detected using a fluorescence microplate reader.

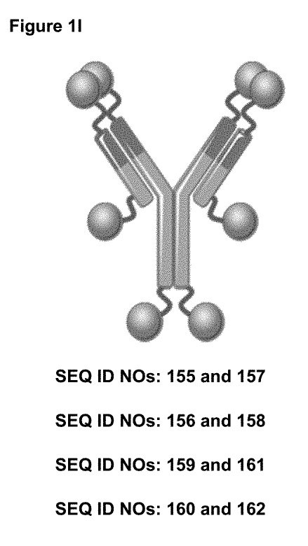

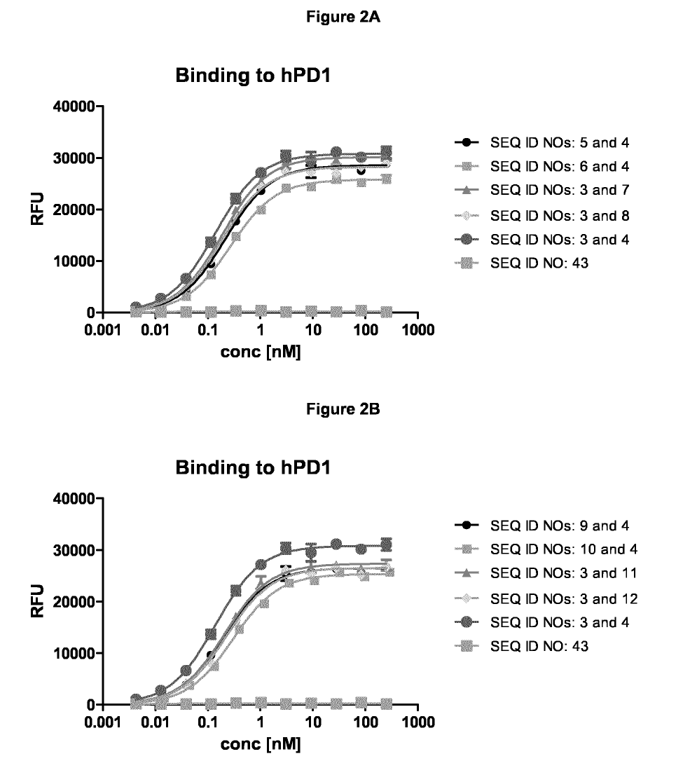

The result of the experiment is depicted in FIG. 2, together with the fit curves resulting from a 1:1 binding sigmoidal fit, where the EC₅₀ value and the maximum signal were free parameters, and the slope was fixed to unity. The resulting EC₅₀ values are provided in Table 2. The observed EC₅₀ values for all tested molecules were very similar and were comparable to the PD-1-specific antibody (SEQ ID NOs: 3 and 4) included in the fusion polypeptides. The experiment shows that when included in fusion polypeptides the described PD-1-specific antibody can be fused with the lipocalin mutein at either one of the four termini of the antibody and still binds to PD-1.

TABLE 2
ELISA data for PD-1 binding

| | EC₅₀ PD-1 |
|---|---|
| Clone Name | [nM] |
| SEQ ID NOs: 5 and 4 | 0.22 |
| SEQ ID NOs: 6 and 4 | 0.26 |
| SEQ ID NOs: 3 and 7 | 0.19 |
| SEQ ID NOs: 3 and 8 | 0.19 |
| SEQ ID NOs: 9 and 4 | 0.20 |
| SEQ ID NOs: 10 and 4 | 0.30 |
| SEQ ID NOs: 3 and 11 | 0.21 |
| SEQ ID NOs: 3 and 12 | 0.23 |
| SEQ ID NOs: 3 and 4 | 0.14 |

### Example 3: Binding of Fusion Polypeptides Towards LAG-3 in ELISA

We employed an ELISA assay to determine the binding potency of the antibody-lipocalin mutein fusion polypeptides, the Fc-lipocalin mutein fusions polypeptides (SEQ ID NO: 41 and SEQ ID NO: 42) and the parental lipocalin muteins of SEQ ID NO: 17 and SEQ ID NO: 27 to recombinant LAG-3-His (ACROBiosystems). The fusion polypeptides/lipocalin muteins were diluted in PBS (1 μg/mL) and coated overnight on microtiter plates at 4° C. The plates were washed after each incubation step with 100 μL PBS-0.05% T five times. The plates were blocked with 2% BSA (w/v) in PBS-0.1% T for 1 h at room temperature and subsequently washed. Different concentrations of the LAG-3-specific lipocalin muteins (SEQ ID NO: 17 and SEQ ID NO: 27) in monomeric form or the antibody-lipocalin mutein fusion polypeptides or Fc-lipocalin mutein polypeptides were added to the wells and incubated for 1 h at room temperature, followed by another wash step. A polyclonal 1:2000 diluted anti-Tic antibody conjugated to HRP in PBS-0.1% T-2% BSA was added for 1 h at room temperature after 1 h incubation. After an additional wash step, fluorogenic HRP substrate (QuantaBlu, Thermo) was added to each well and the fluorescence intensity was detected using a fluorescence microplate reader. Also, in separate experiments, a 1:5000 diluted anti-human IgG Fc-HRP (Jackson Laboratory) was added in the otherwise identical ELISA assay.

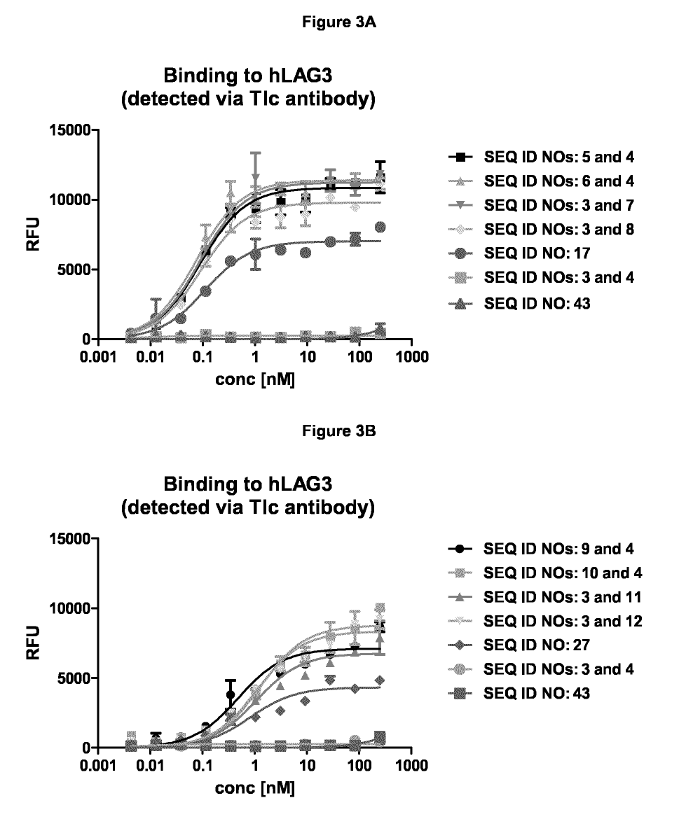

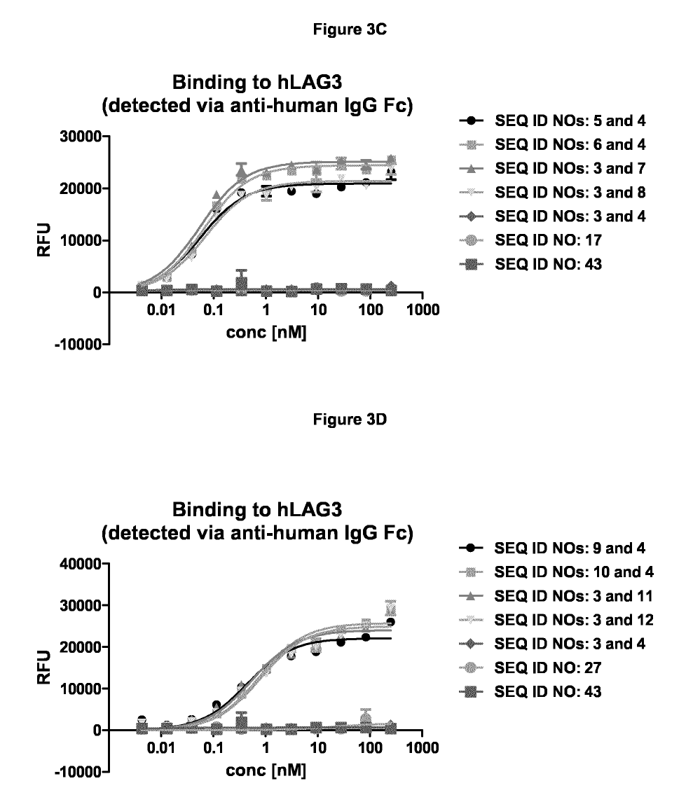

The results of the experiments are depicted in FIG. 3, together with the fit curves resulting from a 1:1 binding sigmoidal fit, where the EC₅₀ value and the maximum signal were free parameters, and the slope was fixed to unity. The resulting EC₅₀ values are shown in Table 3. EC₅₀ values for tested molecules were comparable for both detection methods. The binding potencies to LAG-3 for the different fusion formats when the same lipocalin mutein was included in the polypeptides were comparable with each other and with the respective parental lipocalin mutein. SEQ ID NO: 43 served as a negative control and did not show binding to LAG-3 (data not shown). The experiment shows that when included in fusion polypeptides described above the lipocalin mutein can be fused to the four termini of the antibody without a loss in activity towards LAG-3.

TABLE 3
ELISA data for LAG-3 binding.

| | Detection via | Detection via |
|---|---|---|
| | anti-Tlc | anti-hulgG Fc |
| Clone Name | EC₅₀ LAG-3 [nM] | EC₅₀ LAG-3 [nM] |
| SEQ ID NOs: 5 and 4 | 0.09 | 0.06 |
| SEQ ID NOs: 6 and 4 | 0.08 | 0.07 |
| SEQ ID NOs: 3 and 7 | 0.09 | 0.05 |
| SEQ ID NOs: 3 and 8 | 0.09 | 0.07 |
| SEQ ID NO: 17 | 0.11 | n.a. |
| SEQ ID NOs: 9 and 4 | 0.48 | 0.44 |
| SEQ ID NOs: 10 and 4 | 1.3 | 0.77 |
| SEQ ID NOs: 3 and 11 | 1.1 | 0.55 |
| SEQ ID NOs: 3 and 12 | 1.1 | 0.81 |
| SEQ ID NO: 27 | 0.8 | n.a. |
| SEQ ID NO: 41 | 0.09 | 0.07 |
| SEQ ID NO: 42 | 1.5 | 1.1 |

### Example 4: Fluorescence-Activated Cell Sorting (FACS) Analysis of Fusion Polypeptides Binding to Cells Expressing PD-1 and LAG-3

We employed fluorescence-activated cell sorting (FACS) studies in order to assess the specific binding of fusion polypeptides versus negative controls to Chinese hamster ovary (CHO) cells stably transfected with human PD-1 (CHO-huPD-1) or human LAG-3 (CHO-huLAG-3), respectively. The cell lines were generated using the Flp-In system (Invitrogen) according to the manufacturer's instructions. Mock-transfected Flp-In CHO cells served as the negative control.

Transfected CHO cells were maintained in Ham's F12 medium (Invitrogen) supplemented with 10% Fetal Calf Serum (FCS, Biochrom) and 500 μg/ml Hygromycin B (Roth). Cells were cultured in cell culture flasks under standard conditions according to manufacturer's instruction (37° C., 5% CO₂ atmosphere). In order to dissociate the adherent cells for subculture or FACS experiments, Accutase (PAA Laboratories) was employed according to the manufacturer's instructions.

To perform the experiment, PD-1-positive and negative control Flp-In CHO cells, as well as LAG-3 positive and negative control Flp-In CHO cells were incubated with fusion polypeptides, and bound fusion polypeptides were detected by using a fluorescently labeled anti-lipocalin mutein antibody in FACS analysis as described in the following.

2.5×10⁴ cells per well were pre-incubated for 1 h in ice-cold PBS containing 5% fetal calf serum (PBS-FCS). Subsequently, a dilution series of the fusion polypeptides, lipocalin muteins and negative controls typically ranging from 250 to 0.001 nM was added to the cells, and incubated on ice for 1 h. Cells were washed twice in ice-cold PBS using centrifugation at 300×g and then incubated with a rabbit anti-Tic antibody labelled with the fluorescent dye Alexa488 (Pieris) for 30 min on ice. Cells were subsequently washed and analyzed using iQue Flow cytometer (Intellicyte). The geometric means of the fluorescence intensity were normalized to maximal mean and fit with a 1:1 binding model with EC50 value as free parameter and a slope that was fixed to unity using GraphPad software.

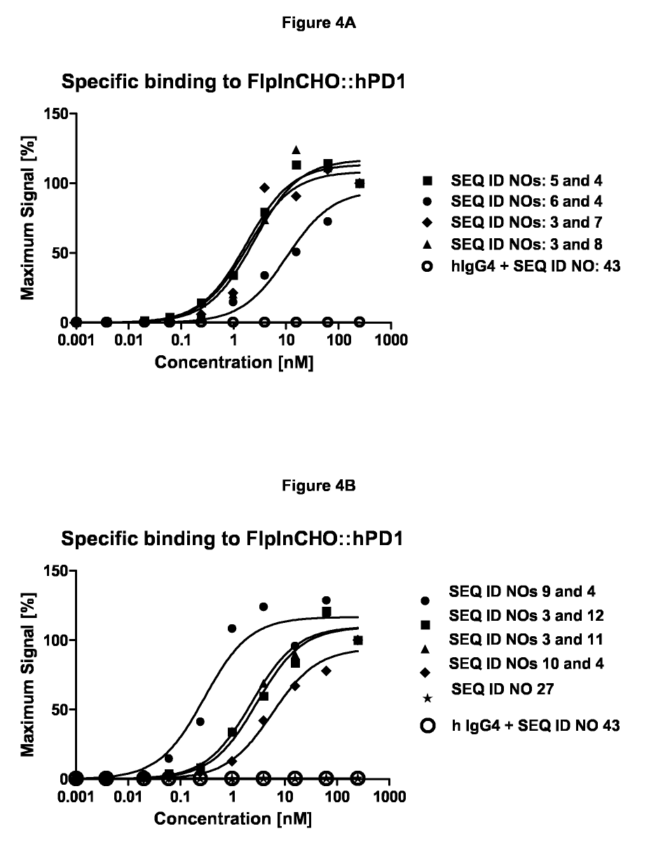

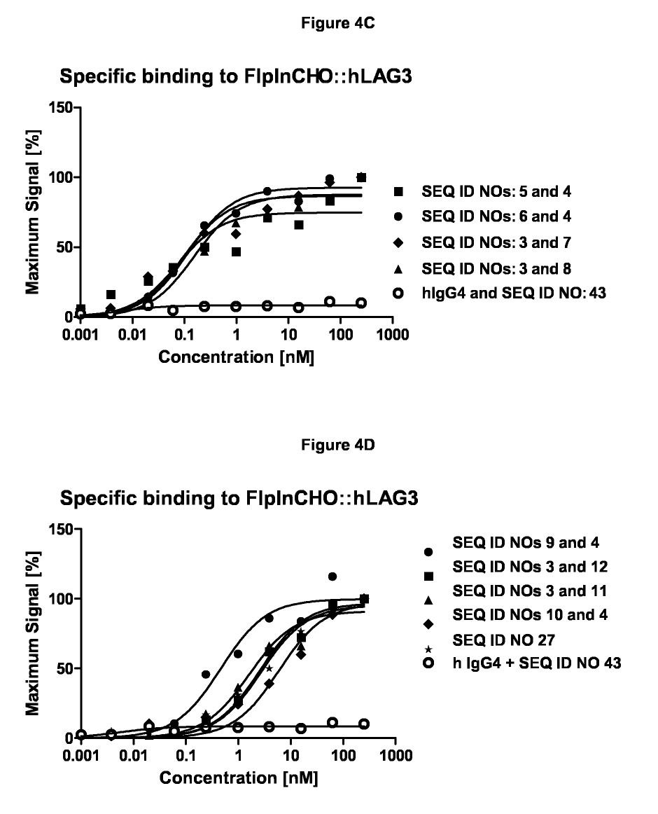

Exemplary data for SEQ ID NOs: 5 and 4, SEQ ID NOs: 6 and 4, SEQ ID NOs: 3 and 7 and SEQ ID NOs: 3 and 8 are shown in FIG. 4 and Table 4. Fusion of the lipocalin mutein to the N-terminus of the anti-PD-1 antibody heavy chain (SEQ ID NOs: 6 and 4) seems to reduce binding potency of the antibody to PD-1, whereas the other fusion sites do not result in a difference in binding to human PD-1 expressed on cells. The improved EC₅₀ to LAG-3 of SEQ ID NOs: 5 and 4 might be due to an avidity effect. Negative controls did not bind to human PD-1 nor human LAG-3 expressed on cells (data not shown) as expected.

TABLE 4
FACS data for binding to huPD-1 and huLAG-3

| | huPD-1 | huLAG-3 |
|---|---|---|
| Clone Name | EC₅₀: [nM] | EC₅₀: [nM] |
| SEQ ID NOs: 5 and 4 | 1.7 | 0.05 |
| SEQ ID NOs: 6 and 4 | 10.5 | 0.13 |
| SEQ ID NOs: 3 and 7 | 1.8 | 0.19 |
| SEQ ID NOs: 3 and 8 | 2.4 | 0.12 |
| SEQ ID NOs: 9 and 4 | 0.28 | 0.60 |
| SEQ ID NOs: 10 and 4 | 5.7 | 7.5 |
| SEQ ID NOs: 3 and 11 | 2.3 | 3.1 |
| SEQ ID NOs: 3 and 12 | 2.9 | 2.0 |

### Example 5: Demonstration of Simultaneous Target Binding in an ELISA-Based Setting

In order to demonstrate the simultaneous binding of the fusion polypeptides to PD-1 and LAG-3, a dual-binding ELISA format was used. Recombinant PD-1-His (ACROBiosystems) in PBS (1 μg/mL) was coated overnight on microtiter plates at 4° C. The plates were washed five times after each incubation step with 100 μL PBS-0.05% T. The plates were blocked with 2% BSA (w/v) in PBS-0.1% T for 1 h at room temperature and subsequently washed again. Different concentrations of the fusion polypeptides were added to the wells and incubated for 1 h at room temperature, followed by a wash step. Subsequently, biotinylated human LAG-3-Fc (R&D Systems) was added at a constant concentration of 2 μg/mL in PBS-0.1% T-2% BSA for 1 h. After washing, 1:5000 dilution of Extravidin-HRP (Sigma-Aldrich) in PBS-0.1% T-2% BSA was added to the wells and incubated for 1 h. After an additional wash step, fluorogenic HRP substrate (QuantaBlu, Thermo) was added to each well and the fluorescence intensity was detected using a fluorescence microplate reader.

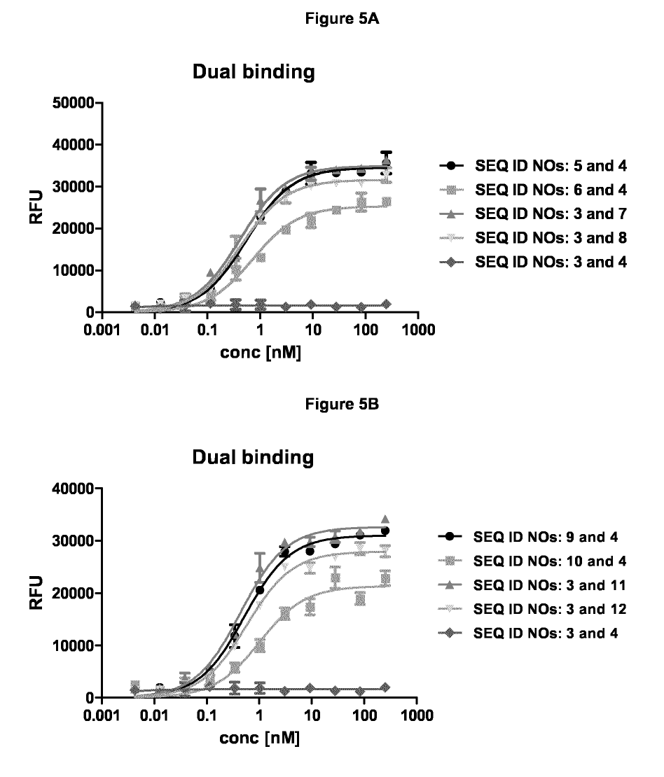

Dual binding data of the fusion polypeptides are shown in FIG. 5, together with the fit curves resulting from a 1:1 binding sigmoidal fit, where the EC₅₀ value and the maximum signal were free parameters, and the slope was fixed to unity. The EC₅₀ values are summarized in Table 5. All fusion polypeptides showed clear binding signals, demonstrating that the fusion polypeptides are able to engage PD-1 and LAG-3 simultaneously. However, the attachment point of the lipocalin mutein on the antibody has an impact on the EC₅₀ in this dual-binding format, as the N-terminal heavy chain fusions (SEQ ID NOs: 6 and 4 and SEQ ID NOs: 10 and 4) have 2 fold reduced EC₅₀s compared to other formats.

TABLE 5
ELISA data for simultaneous target
binding of both PD-1 and LAG-3

| Clone Name | EC₅₀ dual binding [nM] |
|---|---|
| SEQ ID NOs: 5 and 4 | 0.55 |
| SEQ ID NOs: 6 and 4 | 0.75 |
| SEQ ID NOs: 3 and 7 | 0.42 |
| SEQ ID NOs: 3 and 8 | 0.43 |
| SEQ ID NOs: 9 and 4 | 0.52 |
| SEQ ID NOs: 10 and 4 | 1 |
| SEQ ID NOs: 3 and 11 | 0.45 |
| SEQ ID NOs: 3 and 12 | 0.59 |
| SEQ ID NOs: 3 and 4 | No dual binding |

### Example 6: FACS Analysis of Competitive Binding of Fusion Polypeptides with Major Histocompatibility Complex (MHC) Class II Expressing Cells for Human LAG-3

To assess whether a given fusion polypeptide interferes with LAG-3 binding to major histocompatibility complex (MHC) class II on MHC class II-positive cells, a competition FACS experiment was utilized. In this experiment, a constant concentration of human LAG-3-Fc fusion (huLAG-3-Fc, R&D system) and a dilution series of the fusion polypeptides were incubated with the MHC class II positive human cell line A375, and cell-bound huLAG-3-Fc was detected using a fluorescently labelled anti-IgG Fc antibody. In this assay, competitive lipocalin muteins interfering with the binding of huLAG-3 with its ligand MHC class II leads to a reduction of huLAG-3-Fc binding to the MHC class II positive cell line A375.

The melanoma cell line A375 was maintained in DMEM medium (Invitrogen) supplemented with 10% Fetal Calf Serum (FCS, Biochrom). Cells were cultured in cell culture flasks under standard conditions according to manufacturer's instruction (37° C., 5% CO₂ atmosphere). In order to dissociate the adherent cells for subculture or FACS experiments, Accutase (PAA Laboratories) was employed according to the manufacturer's instructions.

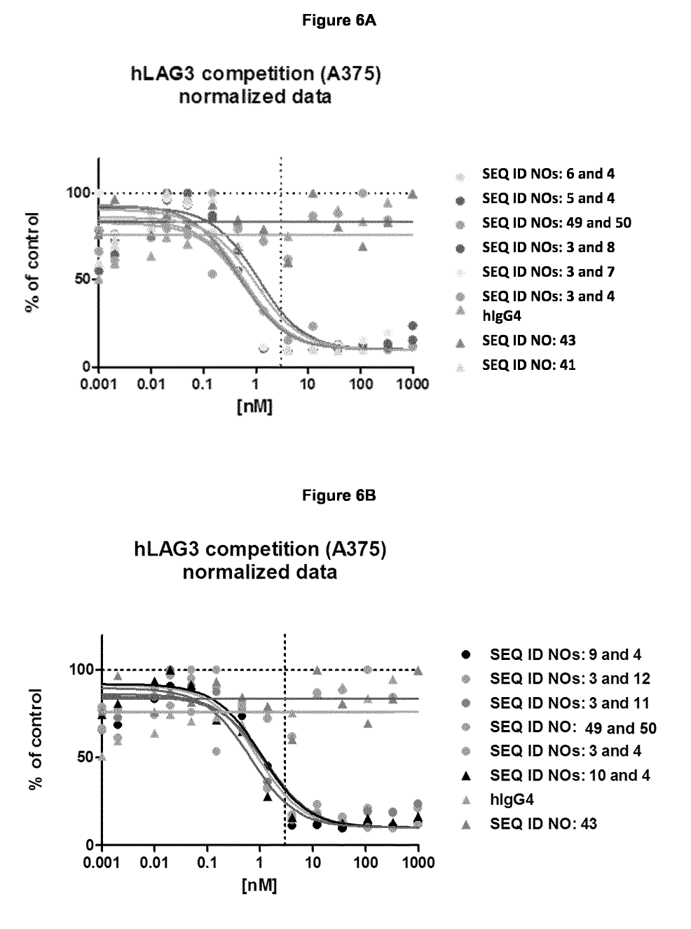

For FACS assay, 5×10⁴ A375 cells per well were incubated for 1 h in PBS-FCS, followed by addition of 3 nM huLAG-3-Fc and varying concentrations of the fusion polypeptides. Cells were washed twice in ice-cold PBS, re-suspended in PBS-FCS and incubated 30 min on ice with phycoerythrin labelled anti-human IgG Fc antibody (Jackson Immunologics). Cells were subsequently washed and analyzed using a Intellicyt IQue Flow cytometer (Intellicyt). Fluorescent data generated by huLAG-3-Fc binding to A375 cells were analyzed using Forecyt software, and resulted geometric fluorescent mean were normalized to huLAG-3-Fc maximal binding. Percent of huLAG-3-Fc binding were plotted and fitted using Graphpad software. Selected competition binding curves are provided in FIG. 6. The data show that the antibody-lipocalin mutein fusion polypeptides and the Fc-lipocalin mutein fusion polypeptides tested compete with binding of huLAG-3 to its ligand MHC class II on human MHC class II expressing cells. The inhibitory effect on LAG-3/MHC class II molecules binding of the fusion polypeptides appeared at concentrations comparable to the reference LAG-3 monoclonal antibody (SEQ ID NOs: 49 and 50). The negative controls hIgG4 (Sigma) and lipocalin mutein (SEQ ID NO: 43), which did not bind to LAG-3, did not show any competition, see FIG. 6.

### Example 7: Assessment of T Cell Activation Using Human Peripheral Blood Mononuclear Cells (PBMCs)

We employed a T cell assay to assess the ability of the fusion polypeptides to revert the inhibitory signaling of the negative checkpoint molecules LAG-3 and PD-1 by blocking the interaction between LAG-3 and PD-1 and the respective ligands. For this purpose, fusion polypeptides at different concentrations were added to staphylococcal enterotoxin B (SEB) stimulated human peripheral blood mononuclear cells (PBMCs) and incubated for 3 days at 37° C. As readouts secreted IL-2 and IFN-γ levels in the supernatants were assessed.

PBMCs from healthy volunteer donors were isolated from buffy coats by centrifugation through a polysucrose density gradient (Biocoll, 1.077 g/mL, Biochrom), following Biochrom's protocols. The purified PBMCs were resuspended in a buffer consisting of 90% FCS and 10% DMSO, immediately frozen down using liquid nitrogen and stored in liquid nitrogen until further use. For the assay, PBMCs were thawed for 16 h and cultivated in culture media (RPMI 1640, Life Technologies) supplemented with 10% FCS and 1% Penicillin-Streptomycin (Life Technologies).

The following procedure was performed using triplicates for each experimental condition.

1×10⁵ PBMCs were incubated in each well of a flat-bottom tissue culture plates in culture media supplemented or not with SEB at different concentrations. The fusion polypeptides are subsequently added to the wells at two different concentrations, i.e. 150 nM or 2000 nM. Plates were covered with a gas permeable seal (4titude) and incubated at 37° C. in a humidified 5% CO₂ atmosphere for 3 days. Subsequently, IL-2 and IFN-γ levels in the supernatant were assessed.

Human IL-2 and human IFN-γ in the cell culture supernatants were quantified using the IL-2 and the IFN-γ DuoSet kit from R&D Systems.

The following procedure describes the IL-2 quantification. The same procedure was used for IFN-γ quantification using specific IFN-γ antibodies.

In the first step, a 384 well plate was coated at room temperature for 2 h with 1 μg/mL “Human IL-2 Capture Antibody” (R&D Systems) in PBS. Subsequently, wells were washed 5 times with 80 μl PBS-0.05% T. After 1 h blocking in PBS-0.05% T additionally containing 1% casein (w/w), pooled supernatants and a concentration series of an IL-2 standard diluted in culture medium was incubated in the 384-well plate overnight at 4° C. To allow for detection and quantitation of captured IL-2, a mixture of 100 ng/mL goat anti-hIL-2-Bio detection antibody (R&D Systems) and 1 μg/mL Sulfotag-labelled streptavidin (Mesoscale Discovery) in PBS-T containing 0.5% casein were added, and incubated at room temperature for 1 h. After washing, 25 μL reading buffer was added to each well and the electrochemiluminescence (ECL) signal of every well was read using a Mesoscale Discovery reader. Analysis and quantification was performed using Mesoscale Discovery software.

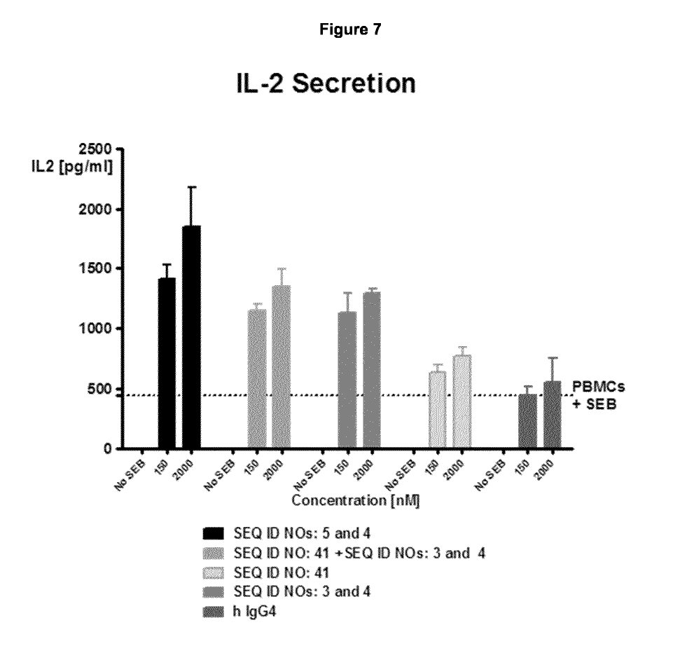

The result of a representative experiment is depicted in FIG. 7. It shows the increased IL-2 secretion level induced by the fusion polypeptide (SEQ ID NOs: 5 and 4). The fusion polypeptide shows improved cytokine secretion, thus T cells activation than the benchmark antibody/lipocalin-Fc mutein cocktail (SEQ ID NOs: 3 and 4 and SEQ ID NO: 41), the PD-1-specific benchmark antibody (SEQ ID NOs: 3 and 4) included in the fusion polypeptides, or the lipocalin-Fc mutein. The negative controls of hIgG4 (Sigma) barely induces further IL-2 production by T cells than basal activity.

### Example 8: Functional T Cell Activation Assay Using A375 Tumor Cells Expressing LAG-3 and PD-1 Ligands

We employed a further T cell assay to assess the ability of the fusion polypeptides revert the inhibitory signaling of the negative checkpoint molecules LAG-3 and PD-1 by blocking the interaction of LAG-3 and PD-1 with their respective ligands. We applied fusion polypeptides at different concentrations to PHA pre-stimulated T cells, in the presence of the melanoma cell line A375 which expresses MHC II, the ligand of LAG-3, and PD-L1, the ligand of PD-1, followed by 3-day incubation at 37° C. As readouts, we assessed secreted IL-2 and IFN-γ levels in the supernatants.

Human peripheral blood mononuclear cells (PBMC) from healthy volunteer donors were isolated from buffy coats by centrifugation through a Polysucrose density gradient (Biocoll 1.077 g/mL from Biochrom), following Biochrom's protocols. The T lymphocytes were isolated from the resulting PBMC using a Pan T cell purification Kit (Miltenyi Biotec GmbH) and the manufacturer's protocols. Purified T cells were resuspended in a buffer consisting of 90% FCS and 10% DMSO, immediately frozen down using liquid nitrogen and stored in liquid nitrogen until further use.

For the assay, T cells were thawed for 16 h and cultivated in culture media (RPMI 1640, Life Technologies) supplemented with 10% FCS and 1% Penicillin-Streptomycin (Life Technologies). T cells were then set at the density of 2×10⁶ cells/ml, and stimulated for 48 h with 5 μg/ml PHA-P (Sigma Aldrich) in culture media.

The following procedure was performed using triplicates for each experimental condition.

Melanoma cell line A375 was plated at 5×10⁴ cells per well and allowed to adhere overnight at 37° C. in a humidified 5% CO₂ atmosphere. The target cells had before been grown under standard conditions, detached using Accutase (PAA Laboratories), and resuspended in culture media.

On the next days, tumor cells were treated 1 hour at 37° C. with mitomycin C (Sigma Aldrich) at a concentration of 30 μg/ml in order to block their proliferation. Plates were washed twice with PBS, and 100 μL of the PHA prestimulated T cell suspension (corresponding to 5×10⁴ T cells), the selected fusion polypeptide (SEQ ID NOs: 5 and 4), antibody/lipocalin mutein cocktail, PD-1-specific benchmark antibody (SEQ ID NOs: 3 and 4), or the negative controls, at concentrations ranging from 1 nM to 100 nM, were added to each well. Plates were covered with a gas permeable seal (4titude) and incubated at 37° C. in a humidified 5% CO₂ atmosphere for 3 days.

Subsequently, IL-2 and IFN-γ levels in the supernatant were assessed as described in Example 7 for IFN-γ secretion (data on IL-2 secretion are not shown).

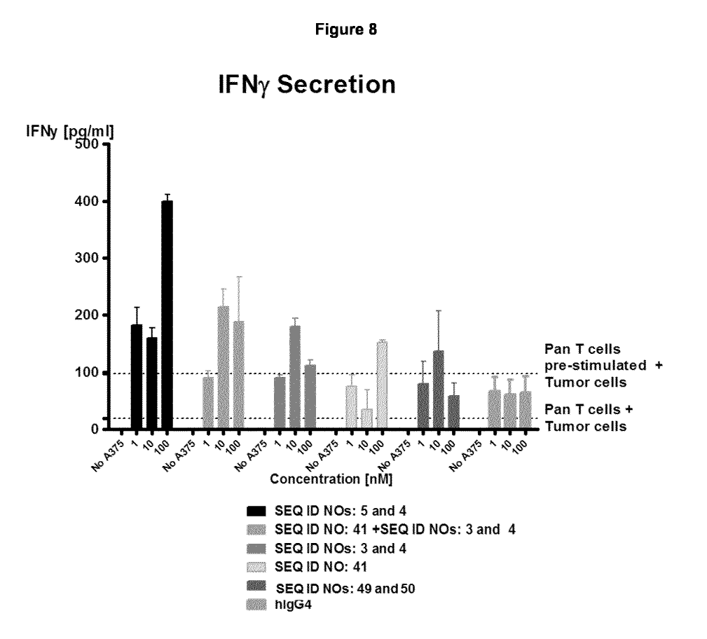

Exemplary data shown in FIG. 8. These data indicate a clear increase of IFN-γ secretion levels with the treatment of the PD-1 and LAG-3 bispecific fusion polypeptides.

### Example 9: Stability Assessment of the Fusion Polypeptides

To determine melting temperatures (Tₘs) as a general indicator for overall stability, the fusion polypeptides at a protein concentration of 1 mg/mL in PBS (Gibco) were scanned (25-100° C.) at 1° C./min using a capillary nanoDSC instrument (CSC 6300, TA Instruments). The Tₘs were calculated from the displayed thermogram using the integrated Nano Analyze software.

The resulting Tₘs as well as the onset of melting for the fusion polypeptides are listed in Table 6 below. All fusion polypeptides have Tₘs as well as onset of melting in the same range as the reference antibody (SEQ ID NOs: 3 and 4).

TABLE 6
Melting temperature (Tₘ) and onset of melting
of fusion polypeptides as determined by nanoDSC

nanoDSC

| SEQ ID | Tₘ [° C.] | onset |
|---|---|---|
| SEQ ID NOs: 9 and 4 | 67 and 68 | 62 |
| SEQ ID NOs: 10 and 4 | 66 and 72 | 59 |
| SEQ ID NOs: 3 and 11 | 64 and 67 and 72 | 57 |
| SEQ ID NOs: 3 and 12 | 67 and 71 | 60 |
| SEQ ID NOs: 5 and 4 | 68 and 72 | 61 |
| SEQ ID NOs: 6 and 4 | 68 and 73 | 62 |
| SEQ ID NOs: 3 and 7 | 68 and 72 | 61 |
| SEQ ID NOs: 3 and 8 | 68 and 73 | 62 |
| SEQ ID NOs: 3 and 4 | 69 | 62 |

To assess storage stability, the fusion polypeptides were incubated at a concentration of 1 mg/mL in PBS for 1 week at 37° C. Active fusion polypeptide was measured in a quantitative ELISA (qELISA) setting. Monomeric protein was measured in an analytical size exclusion chromatography. Exemplary data for SEQ ID NOs: 5 and 4, SEQ ID NOs: 6 and 4, SEQ ID NOs: 3 and 7 and SEQ ID NOs: 3 and 8 are shown in Table 8.

For assaying protein activity, the simultaneous binding ELISA as described in Example 5 was applied.

A calibration curve with standard protein dilutions was prepared. Three different, independent dilutions within the linear range of the calibration curve were prepared for each sample. PBS-0.1% T-2% BSA optionally supplemented with 1% human plasma was used for the dilutions. The percentage recovery of activity for each sample was calculated from the calibration curve, referencing against an unstressed sample stored at −20° C. at the same concentration and in the same matrix.

Analytical size exclusion chromatography was performed on an Agilent HPLC system with two Superdex 200, 3.2/300 increase (GE Healthcare) in a row with PBS (Gibco) as an eluent at a flow rate of 0.3 mL/min. The percentage recovery of monomer was determined by the monomer peak area for each sample referencing against non-stressed reference sample frozen at −20° C.

To further assess the storage stability in plasma, fusion polypeptides at the concentration of 0.5 mg/mL were incubated for 1 week at 37° C. in human plasma. Active fusion polypeptide was measured in a quantitative ELISA setting as described.

TABLE 8
Stability after 1-week storage in PBS or human plasma
(HPL) at 37° C. assessed by recovery of activity
in qELISA and monomer content in analytical SEC (only
for samples stored in PBS): stable in qELISA =
100 +/− 15%; stable in aSEC = 100 +/− 5%;
for all samples including references a monomer content
of at least 99 area percent has been detected.

| | | | 1 week HPL, |
|---|---|---|---|
| | 1 week PBS, 37° C., | | 37° C., |
| | 1 mg/ml | | 0.5 mg/ml |
| | % recovery of | % recovery of | % recovery of |
| | activity | monomer peak | activity |
| SEQ ID | in qELISA | in aSEC | in qELISA |
| SEQ ID NOs: 5 and 4 | 98 | 103% | 99 |
| SEQ ID NOs: 6 and 4 | 105 | 101% | 103 |
| SEQ ID NOs: 3 and 7 | 107 | 104% | 104 |
| SEQ ID NOs: 3 and 8 | 99 | 101% | 99 |

Embodiments illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks, and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

### EQUIVALENTS

Those skilled in the art will recognize, or be able to ascertain using no more than routine experimentation, many equivalents to the specific embodiments of the invention described herein. Such equivalents are intended to be encompassed by the following claims. All publications, patents, and patent applications mentioned in this specification are herein incorporated by reference into the specification to the same extent as if each individual publication, patent or patent application was specifically and individually indicated to be incorporated herein by reference.

### NON-PATENT REFERENCES

1. TRIEBEL, F., JITSUKAWA, S., BAIXERAS, E., ROMAN-ROMAN, S., GENEVEE, C., VIEGAS-PEQUIGNOT, E. & HERCEND, T. 1990. LAG-3, a novel lymphocyte activation gene closely related to CD4. J Exp Med, 171, 1393-405.
2. KISIELOW, M., KISIELOW, J., CAPOFERRI-SOLLAMI, G. & KARJALAINEN, K. 2005. Expression of lymphocyte activation gene 3 (LAG-3) on B cells is induced by T cells. Eur J Immunol, 35, 2081-8.
3. WORKMAN, C. J., WANG, Y., EL KASMI, K. C., PARDOLL, D. M., MURRAY, P. J., DRAKE, C. G. & VIGNALI, D. A. 2009. LAG-3 regulates plasmacytoid dendritic cell homeostasis. J Immunol, 182, 1885-91.
4. HUARD, B., MASTRANGELI, R., PRIGENT, P., BRUNIQUEL, D., DONINI, S., EL-TAYAR, N., MAIGRET, B., DREANO, M. & TRIEBEL, F. 1997. Characterization of the major histocompatibility complex class II binding site on LAG-3 protein. Proc Natl Acad Sci US A, 94, 5744-9.
5. BUISSON, S. & TRIEBEL, F. 2003. MHC class II engagement by its ligand LAG-3 (CD223) leads to a distinct pattern of chemokine and chemokine receptor expression by human dendritic cells. Vaccine, 21, 862-8.
6. ANDREAE, S., PIRAS, F., BURDIN, N. & TRIEBEL, F. 2002. Maturation and activation of dendritic cells induced by lymphocyte activation gene-3 (CD223). J Immunol, 168, 3874-80.
7. MACON-LEMAITRE, L. & TRIEBEL, F. 2005. The negative regulatory function of the lymphocyte-activation gene-3 co-receptor (CD223) on human T cells. Immunology, 115, 170-8.
8. WOO, S. R., TURNIS, M. E., GOLDBERG, M. V., BANKOTI, J., SELBY, M., NIRSCHL, C. J., BETTINI, M. L., GRAVANO, D. M., VOGEL, P., LIU, C. L., TANGSOMBATVISIT, S., GROSSO, J. F., NETTO, G., SMELTZER, M. P., CHAUX, A., UTZ, P. J., WORKMAN, C. J., PARDOLL, D. M., KORMAN, A. J., DRAKE, C. G. & VIGNALI, D. A. 2012. Immune inhibitory molecules LAG-3 and PD-1 synergistically regulate T cell function to promote tumoral immune escape. Cancer Res, 72, 917-27.
9. SHARPE, A. H., WHERRY, E. J., AHMED, R. & FREEMAN, G. J. 2007. The function of programmed cell death 1 and its ligands in regulating autoimmunity and infection. Nat Immunol, 8, 239-45.
10. GREENWALD, R. J., FREEMAN, G. J. & SHARPE, A. H. 2005. The B7 family revisited. Annu Rev Immunol, 23, 515-48.
11. PARRY, R. V., CHEMNITZ, J. M., FRAUWIRTH, K. A., LANFRANCO, A. R., BRAUNSTEIN, I., KOBAYASHI, S. V., LINSLEY, P. S., THOMPSON, C. B. & RILEY, J. L. 2005. CTLA-4 and PD-1 receptors inhibit T cell activation by distinct mechanisms. Mol Cell Biol, 25, 9543-53.
12. GALON, J., COSTES, A., SANCHEZ-CABO, F., KIRILOVSKY, A., MLECNIK, B., LAGORCE-PAGES, C., TOSOLINI, M., CAMUS, M., BERGER, A., WIND, P., ZINZINDOHOUE, F., BRUNEVAL, P., CUGNENC, P. H., TRAJANOSKI, Z., FRIDMAN, W. H. & PAGES, F. 2006. Type, density, and location of immune cells within human colorectal tumors predict clinical outcome. Science, 313, 1960-4.
13. DONG, H., STROME, S. E., SALOMAO, D. R., TAMURA, H., HIRANO, F., FLIES, D. B., ROCHE, P. C., LU, J., ZHU, G., TAMADA, K., LENNON, V. A., CELIS, E. & CHEN, L. 2002. Tumor-associated B7-H1 promotes T cell apoptosis: a potential mechanism of immune evasion. Nat Med, 8, 793-800.
14. BLANK, C., BROWN, I., PETERSON, A. C., SPIOTTO, M., IWAI, Y., HONJO, T. & GAJEWSKI, T. F. 2004. PD-L1/B7H-1 inhibits the effector phase of tumor rejection by T cell receptor (TCR) transgenic CD8+ T cells. Cancer Res, 64, 1140-5.
15. IWAI, Y., TERAWAKI, S. & HONJO, T. 2005. PD-1 blockade inhibits hematogenous spread of poorly immunogenic tumor cells by enhanced recruitment of effector T cells. Int Immunol, 17, 133-44.
16. HIRANO, F., KANEKO, K., TAMURA, H., DONG, H., WANG, S., ICHIKAWA, M., RIETZ, C., FLIES, D. B., LAU, J. S., ZHU, G., TAMADA, K. & CHEN, L. 2005. Blockade of B7-H1 and PD-1 by monoclonal antibodies potentiates cancer therapeutic immunity. Cancer Res, 65, 1089-96.
17. HAMANISHI, J., MANDAI, M., IWASAKI, M., OKAZAKI, T., TANAKA, Y., YAMAGUCHI, K., HIGUCHI, T., YAGI, H., TAKAKURA, K., MINATO, N., HONJO, T. & FUJII, S. 2007. Programmed cell death 1 ligand 1 and tumor-infiltrating CD8+ T lymphocytes are prognostic factors of human ovarian cancer. Proc Natl Acad Sci USA, 104, 3360-5.
18. ALTSCHUL, S. F., MADDEN, T. L., SCHAFFER, A. A., ZHANG, J., ZHANG, Z., MILLER, W. & LIPMAN, D. J. 1997. Gapped BLAST and PSI-BLAST: a new generation of protein database search programs. Nucleic Acids Res, 25, 3389-402.
19. ALTSCHUL, S. F., GISH, W., MILLER, W., MYERS, E. W. & LIPMAN, D. J. 1990. Basic local alignment search tool. J Mol Biol, 215, 403-10.
20. SMITH, T. F. & WATERMAN, M. S. 1981. Identification of common molecular subsequences. J Mol Biol, 147, 195-7.
21. JOHNSON, G. & WU, T. T. 2000. Kabat database and its applications: 30 years after the first variability plot. Nucleic Acids Res, 28, 214-8.
22. SHIELDS, R. L., NAMENUK, A. K., HONG, K., MENG, Y. G., RAE, J., BRIGGS, J., XIE, D., LAI, J., STADLEN, A., LI, B., FOX, J. A. & PRESTA, L. G. 2001. High resolution mapping of the binding site on human IgG1 for Fc gamma RI, Fc gamma RII, Fc gamma RIII, and FcRn and design of IgG1 variants with improved binding to the Fc gamma R. J Biol Chem, 276, 6591-604.
23. ALTSHULER, E. P., SEREBRYANAYA, D. V. & KATRUKHA, A. G. 2010. Generation of recombinant antibodies and means for increasing their affinity. Biochemistry (Mosc), 75, 1584-605.
24. HARLOW, E. & LANE, D. 1999. Using antibodies: a laboratory manual, Cold Spring Harbor, N.Y., Cold Spring Harbor Laboratory Press.
25. HARLOW, E. & LANE, D. 1988. Antibodies: a laboratory manual, Cold Spring Harbor, N.Y., Cold Spring Harbor Laboratory.
26. LI, J., SAI, T., BERGER, M., CHAO, Q., DAVIDSON, D., DESHMUKH, G., DROZDOWSKI, B., EBEL, W., HARLEY, S., HENRY, M., JACOB, S., KLINE, B., LAZO, E., ROTELLA, F., ROUTHIER, E., RUDOLPH, K., SAGE, J., SIMON, P., YAO, J., ZHOU, Y., KAVURU, M., BONFIELD, T., THOMASSEN, M. J., SASS, P. M., NICOLAIDES, N. C. & GRASSO, L. 2006. Human antibodies for immunotherapy development generated via a human B cell hybridoma technology. Proc Natl Acad Sci USA, 103, 3557-62.
27. KOZBOR, D. & RODER, J. C. 1983. The production of monoclonal antibodies from human lymphocytes. Immunol Today, 4, 72-9.
28. COLE, S. P., CAMPLING, B. G., LOUWMAN, I. H., KOZBOR, D. & RODER, J. C. 1984. A strategy for the production of human monoclonal antibodies reactive with lung tumor cell lines. Cancer Res, 44, 2750-3.
29. HOLLIGER, P. & HUDSON, P. J. 2005. Engineered antibody fragments and the rise of single domains. Nat Biotechnol, 23, 1126-36.
30. SKERRA, A. 2000. Lipocalins as a scaffold. Biochim Biophys Acta, 1482, 337-50.
31. FLOWER, D. R., NORTH, A. C. & SANSOM, C. E. 2000. The lipocalin protein family: structural and sequence overview. Biochim Biophys Acta, 1482, 9-24.
32. FLOWER, D. R. 1996. The lipocalin protein family: structure and function. Biochem J, 318 (Pt 1), 1-14.
33. FLOWER, D. R. 2000. Beyond the superfamily: the lipocalin receptors. Biochim Biophys Acta, 1482, 327-36.
34. BREUSTEDT, D. A., KORNDORFER, I. P., REDL, B. & SKERRA, A. 2005. The 1.8-A crystal structure of human tear lipocalin reveals an extended branched cavity with capacity for multiple ligands. J Biol Chem, 280, 484-93.
35. SAMBROOK, J. & RUSSELL, D. W. 2001. Molecular cloning: a laboratory manual, Cold Spring Harbor, N.Y., Cold Spring Harbor Laboratory Press.
36. WORKMAN, C. J. & VIGNALI, D. A. 2005. Negative regulation of T cell homeostasis by lymphocyte activation gene-3 (CD223). J Immunol, 174, 688-95.
37. HUANG, C. T., WORKMAN, C. J., FLIES, D., PAN, X., MARSON, A. L., ZHOU, G., HIPKISS, E. L., RAVI, S., KOWALSKI, J., LEVITSKY, H. I., POWELL, J. D., PARDOLL, D. M., DRAKE, C. G. & VIGNALI, D. A. 2004. Role of LAG-3 in regulatory T cells. Immunity, 21, 503-13.
38. CAMISASCHI, C., CASATI, C., RINI, F., PEREGO, M., DE FILIPPO, A., TRIEBEL, F., PARMIANI, G., BELLI, F., RIVOLTINI, L. & CASTELLI, C. 2010. LAG-3 expression defines a subset of CD4(+)CD25(high)Foxp3(+) regulatory T cells that are expanded at tumor sites. J Immunol, 184, 6545-51.
39. GROSSO, J. F., KELLEHER, C. C., HARRIS, T. J., MARIS, C. H., HIPKISS, E. L., DE MARZO, A., ANDERS, R., NETTO, G., GETNET, D., BRUNO, T. C., GOLDBERG, M. V., PARDOLL, D. M. & DRAKE, C. G. 2007. LAG-3 regulates CD8+ T cell accumulation and effector function in murine self- and tumor-tolerance systems. J Clin Invest, 117, 3383-92.
40. BLACKBURN, S. D., SHIN, H., HAINING, W. N., ZOU, T., WORKMAN, C. J., POLLEY, A., BETTS, M. R., FREEMAN, G. J., VIGNALI, D. A. & WHERRY, E. J. 2009. Coregulation of CD8+ T cell exhaustion by multiple inhibitory receptors during chronic viral infection. Nat Immunol, 10, 29-37.
41. LYFORD-PIKE, S., PENG, S., YOUNG, G. D., TAUBE, J. M., WESTRA, W. H., AKPENG, B., BRUNO, T. C., RICHMON, J. D., WANG, H., BISHOP, J. A., CHEN, L., DRAKE, C. G., TOPALIAN, S. L., PARDOLL, D. M. & PAI, S. I. 2013. Evidence for a role of the PD-1:PD-L1 pathway in immune resistance of HPV-associated head and neck squamous cell carcinoma. Cancer Res, 73, 1733-41.
42. BETTINI, M., SZYMCZAK-WORKMAN, A. L., FORBES, K., CASTELLAW, A. H., SELBY, M., PAN, X., DRAKE, C. G., KORMAN, A. J. & VIGNALI, D. A. 2011. Cutting edge: accelerated autoimmune diabetes in the absence of LAG-3. J Immunol, 187, 3493-8.
43. OKAZAKI, T., OKAZAKI, I. M., WANG, J., SUGIURA, D., NAKAKI, F., YOSHIDA, T., KATO, Y., FAGARASAN, S., MURAMATSU, M., ETO, T., HIOKI, K. & HONJO, T. 2011. PD-1 and LAG-3 inhibitory co-receptors act synergistically to prevent autoimmunity in mice. J Exp Med, 208, 395-407.
44. GODING, S. R., WILSON, K. A., XIE, Y., HARRIS, K. M., BAXI, A., AKPINARLI, A., FULTON, A., TAMADA, K., STROME, S. E. & ANTONY, P. A. 2013. Restoring immune function of tumor-specific CD4+ T cells during recurrence of melanoma. J Immunol, 190, 4899-909.
45. GANDHI, M. K., LAMBLEY, E., DURAISWAMY, J., DUA, U., SMITH, C., ELLIOTT, S., GILL, D., MARLTON, P., SEYMOUR, J. & KHANNA, R. 2006. Expression of LAG-3 by tumor-infiltrating lymphocytes is coincident with the suppression of latent membrane antigen-specific CD8+ T cell function in Hodgkin lymphoma patients. Blood, 108, 2280-9.
46. MATSUZAKI, J., GNJATIC, S., MHAWECH-FAUCEGLIA, P., BECK, A., MILLER, A., TSUJI, T., EPPOLITO, C., QIAN, F., LELE, S., SHRIKANT, P., OLD, L. J. & ODUNSI, K. 2010. Tumor-infiltrating NY-ESO-1-specific CD8+ T cells are negatively regulated by LAG-3 and PD-1 in human ovarian cancer. Proc Natl Acad Sci USA, 107, 7875-80.
47. KEIR, M. E., BUTTE, M. J., FREEMAN, G. J. & SHARPE, A. H. 2008. PD-1 and its ligands in tolerance and immunity. Annu Rev Immunol, 26, 677-704.
48. DONG, H., ZHU, G., TAMADA, K. & CHEN, L. 1999. B7-H1, a third member of the B7 family, co-stimulates T cell proliferation and interleukin-10 secretion. Nat Med, 5, 1365-9.
49. TERME, M., ULLRICH, E., AYMERIC, L., MEINHARDT, K., DESBOIS, M., DELAHAYE, N., VIAUD, S., RYFFEL, B., YAGITA, H., KAPLANSKI, G., PREVOST-BLONDEL, A., KATO, M., SCHULTZE, J. L., TARTOUR, E., KROEMER, G., CHAPUT, N. & ZITVOGEL, L. 2011. IL-18 induces PD-1-dependent immunosuppression in cancer. Cancer Res, 71, 5393-9.
50. PARDOLL, D. M. 2012. The blockade of immune checkpoints in cancer immunotherapy. Nat Rev Cancer, 12, 252-64.
51. NISHIMURA, H., OKAZAKI, T., TANAKA, Y., NAKATANI, K., HARA, M., MATSUMORI, A., SASAYAMA, S., MIZOGUCHI, A., HIAI, H., MINATO, N. & HONJO, T. 2001. Autoimmune dilated cardiomyopathy in PD-1 receptor-deficient mice. Science, 291, 319-22.
52. NISHIMURA, H., NOSE, M., HIAI, H., MINATO, N. & HONJO, T. 1999. Development of lupus-like autoimmune diseases by disruption of the PD-1 gene encoding an ITIM motif-carrying immunoreceptor. Immunity, 11, 141-51.
53. HAMID, O., ROBERT, C., DAUD, A., HODI, F. S., HWU, W. J., KEFFORD, R., WOLCHOK, J. D., HERSEY, P., JOSEPH, R. W., WEBER, J. S., DRONCA, R., GANGADHAR, T. C., PATNAIK, A., ZAROUR, H., JOSHUA, A. M., GERGICH, K., ELASSAISS-SCHAAP, J., ALGAZI, A., MATEUS, C., BOASBERG, P., TUMEH, P. C., CHMIELOWSKI, B., EBBINGHAUS, S. W., LI, X. N., KANG, S. P. & RIBAS, A. 2013. Safety and tumor responses with lambrolizumab (anti-PD-1) in melanoma. N Engl J Med, 369, 134-44.
54. TOPALIAN, S. L., HODI, F. S., BRAHMER, J. R., GETTINGER, S. N., SMITH, D. C., MCDERMOTT, D. F., POWDERLY, J. D., CARVAJAL, R. D., SOSMAN, J. A., ATKINS, M. B., LEMING, P. D., SPIGEL, D. R., ANTONIA, S. J., HORN, L., DRAKE, C. G., PARDOLL, D. M., CHEN, L., SHARFMAN, W. H., ANDERS, R. A., TAUBE, J. M., MCMILLER, T. L., XU, H., KORMAN, A. J., JURE-KUNKEL, M., AGRAWAL, S., MCDONALD, D., KOLLIA, G. D., GUPTA, A., WIGGINTON, J. M. & SZNOL, M. 2012. Safety, activity, and immune correlates of anti-PD-1 antibody in cancer. N Engl J Med, 366, 2443-54.
55. LOWMAN, H. B. 1997. Bacteriophage display and discovery of peptide leads for drug development. Annu Rev Biophys Biomol Struct, 26, 401-24.
56. RODI, D. J. & MAKOWSKI, L. 1999. Phage-display technology—finding a needle in a vast molecular haystack. Curr Opin Biotechnol, 10, 87-93.
57. VENTURI, M., SEIFERT, C. & HUNTE, C. 2002. High level production of functional antibody Fab fragments in an oxidizing bacterial cytoplasm. J Mol Biol, 315, 1-8.
58. BRUCKDORFER, T., MARDER, O. & ALBERICIO, F. 2004. From production of peptides in milligram amounts for research to multi-tons quantities for drugs of the future. Curr Pharm Biotechnol, 5, 29-43.
59. SILVA, J. P., VETTERLEIN, O., JOSE, J., PETERS, S. & KIRBY, H. 2015. The S228P mutation prevents in vivo and in vitro IgG4 Fab-arm exchange as demonstrated using a combination of novel quantitative immunoassays and physiological matrix preparation. J Biol Chem, 290, 5462-9.

1. A fusion polypeptide that is capable of binding both programmed cell death protein 1 (PD-1) and lymphocyte-activation gene 3 (LAG-3), wherein the fusion polypeptide comprises at least two subunits in any order, wherein the first subunit comprises a full-length immunoglobulin or an antigen-binding domain thereof having binding specificity for PD-1, and wherein the second subunit comprises a lipocalin mutein having binding specificity for LAG-3.

2. (canceled)

3. The fusion polypeptide of claim 1, wherein the fusion polypeptide is capable of binding PD-1 with an EC₅₀ value of at most about 1 nM.

4. The fusion polypeptide of claim 1, wherein the fusion polypeptide is capable of binding PD-1 with and EC₅₀ value of at most about 0.3 nM.

5. (canceled)

6. The fusion polypeptide of claim 1, wherein the fusion polypeptide is capable of binding PD-1 with an EC₅₀ comparable to or lower than the EC₅₀ value of the antibody specific for PD-1 as included in such fusion polypeptide.

7. The fusion polypeptide of claim 1, wherein the fusion polypeptide is capable of binding LAG-3 with an EC₅₀ value of at most about 2 nM.

8. The fusion polypeptide of claim 1, wherein the fusion polypeptide is capable of binding LAG-3 with an EC₅₀ value of at most about 1 nM.

9. The fusion polypeptide of claim 1, wherein the fusion polypeptide is capable of binding LAG-3 with an EC₅₀ value comparable to or lower than the EC₅₀ value of the lipocalin mutein specific for LAG-3 as included in such fusion polypeptide.

10. The fusion polypeptide of claim 1, wherein the fusion polypeptide is capable of binding PD-1 and LAG-3 simultaneously.

11. The fusion polypeptide of claim 1, wherein the fusion polypeptide is capable of simultaneously binding PD-1 and LAG-3 with an EC₅₀ value of at most about 10 nM.

12. The fusion polypeptide of claim 1, wherein the fusion polypeptide is capable of simultaneously binding PD-1 and LAG-3 with an EC₅₀ value of at most about 0.6 nM.

13. (canceled)

14. The fusion polypeptide of claim 1, wherein the fusion polypeptide competitively inhibits the binding of LAG-3 to major histocompatibility complex (MHC) class II.

15. (canceled)

16. The fusion polypeptide of claim 1, wherein the fusion polypeptide is capable of co-stimulating T cell responses.

17. (canceled)

18. The fusion polypeptide of claim 1, wherein the fusion polypeptide is capable of inducing IL-2 and/or IFN-γ production.

19. (canceled)

20. The fusion polypeptide of claim 1, wherein the second subunit is a LAG-3-specific lipocalin mutein comprising one or more mutated amino acid residues at sequence positions corresponding to positions 14, 25-34, 36, 48, 52-53, 55-58, 60-61, 66, 79, 85-86, 101, 104-106, 108, 110-112, 114, 121, 140, and 153 of the linear polypeptide sequence of mature human tear lipocalin (SEQ ID NO: 1).

21. The fusion polypeptide of claim 1, wherein the second subunit is a LAG-3-specific lipocalin mutein comprising at least one of the following mutated amino acid residue in comparison with the linear polypeptide sequence of mature human tear lipocalin (SEQ ID NO: 1): Ser 14→Pro; Asp 25→Ser; Arg 26→Ser, Phe, Gly, Ala, Asp or Glu; Glu 27→Asp, Val or Thr; Phe 28→Cys or Asp; Pro 29→Phe, Leu or Trp; Glu 30→Trp, Asn or Tyr; Met 31→Ile, Val, Asp, Leu or Tyr; Asn 32→Asp, Glu, Tyr, Trp, Val, Thr or Met; Leu 33→Asp, Glu or Pro; Glu 34→Val, Trp or His; Val 36→Ala; Asn 48→Asp; Lys 52→Glu, Ser, Arg or Asn; Val 53→Ala; Met 55→Ala or Val; Leu 56→Asp, Gln or Asn; Ile 57→Leu; Ser 58→Phe, Trp or Asp; Arg 60→Phe or Glu; Cys 61→Trp, Pro, Leu or Trp; Ala 66→Asn; Ala 79→Glu; Val 85→Ala; Ala 86→Asp; Cys 101→Ser or Phe; Glu 104→Tyr; Leu 105→Cys or Gly; His 106→Ala, Glu, Thr, Tyr, Gln or Val; Lys 108→Tyr, Phe, Thr or Trp; Val 110→Gly or Ala; Arg 111→Pro; Gly 112→Met or Thr; Lys 114→Trp or Ala; Lys 121→Thr; Ser 140→Gly; and Cys 153→Ser.

22. The fusion polypeptide of claim 1, wherein the second subunit is a LAG-3-specific lipocalin mutein comprising one of the following sets of mutated amino acid residues in comparison with the linear polypeptide sequence of mature human tear lipocalin (SEQ ID NO: 1):
(a) Arg 26→Ser; Glu 27→Asp; Phe 28→Cys; Pro 29→Phe; Glu 30→Trp; Met 31→Ile; Asn 32→Glu; Leu 33→Glu; Glu 34→Trp; Leu 56→Asp; Ser 58→Phe; Arg 60→Phe; Cys 61→Trp; Cys 101→Ser; Leu 105→Cys; His 106→Ala; Lys 108→Phe; Arg 111→Pro; Lys 114→Trp; and Cys 153→Ser;
(b) Ser 14→Pro; Asp 25→Ser; Arg 26→Gly; Phe 28→Asp; Asn 32→Thr; Lys 52→Asn; Met 55→Ala; Ser 58→Asp; Ala 66→Asn; Ala 79→Glu; Ala 86→Asp; Cys 101→Phe; Leu 105→Gly; Lys 108→Thr; Val 110→Ala; Gly 112→Thr; Lys 114→Ala; and Lys 121→Thr;
(c) Arg 26→Phe; Glu 27→Val; Phe 28→Cys; Pro 29→Leu; Glu 30→Tyr; Met 31→Asp; Asn 32→Val; Leu 33→Pro; Leu 56→Gln; Ser 58→Trp; Arg 60→Glu; Cys 61→Leu; Cys 101→Ser; Glu 104→Tyr; Leu 105→Cys; His 106→Val; Lys 108→Tyr; Arg 111→Pro; Lys 114→Trp; and Cys 153→Ser;
(d) Arg 26→Glu; Glu 27→Thr; Phe 28→Cys; Pro 29→Trp; Glu 30→Trp; Met 31→Tyr; Asn 32→Val; Leu 33→Asp; Glu 34→His; Leu 56→Asn; Ile 57→Leu; Ser 58→Trp; Arg 60→Phe; Cys 61→Trp; Cys 101→Ser; Leu 105→Cys; His 106→Gln; Lys 108→Trp; Arg 111→Pro; Lys 114→Trp; and Cys 153→Ser;
(e) Arg 26→Ser; Glu 27→Asp; Phe 28→Cys; Pro 29→Phe; Glu 30→Trp; Met 31→Ile; Asn 32→Asp; Leu 33→Asp; Glu 34→Val; Leu 56→Asp; Ser 58→Phe; Arg 60→Phe; Cys 61→Trp; Cys 101→Ser; Leu 105→Cys; His 106→Ala; Lys 108→Tyr; Arg 111→Pro; Lys 114→Trp; and Cys 153→Ser;
(f) Arg 26→Ser; Glu 27→Asp; Phe 28→Cys; Pro 29→Phe; Glu 30→Trp; Met 31→Ile; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Ala; Lys 108→Tyr; Arg 111→Pro; Lys 114→Trp; and Cys 153→Ser;
(g) Arg 26→Ser; Glu 27→Asp; Phe 28→Cys; Pro 29→Phe; Glu 30→Trp; Met 31→Ile; Asn 32→Glu; Leu 33→Glu; Glu 34→Trp; Val 36→Ala; Asn 48→Asp; Leu 56→Asp; Ser 58→Phe; Arg 60→Phe; Cys 61→Trp; Val 85→Ala; Cys 101→Ser; Leu 105→Cys; His 106→Ala; Lys 108→Phe; Arg 111→Pro; Lys 114→Trp; Ser 140→Gly; and Cys 153→Ser;
(h) Arg 26→Ser; Glu 27→Asp; Phe 28→Cys; Pro 29→Phe; Glu 30→Trp; Met 31→Ile; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Glu; Lys 108→Phe; Arg 111→Pro; Lys 114→Trp; and Cys 153→Ser;
(i) Arg 26→Ser; Glu 27→Asp; Phe 28→Cys; Pro 29→Phe; Glu 30→Trp; Met 31→Ile; Asn 32→Glu; Leu 33→Glu; Glu 34→Trp; Val 36→Ala; Lys 52→Glu; Val 53→Ala; Leu 56→Asp; Ser 58→Phe; Arg 60→Phe; Cys 61→Trp; Cys 101→Ser; Leu 105→Cys; His 106→Ala; Lys 108→Phe; Arg 111→Pro; Lys 114→Trp; and Cys 153→Ser;
(j) Arg 26→Ser; Glu 27→Asp; Phe 28→Cys; Pro 29→Phe; Glu 30→Trp; Met 31→Val; Asn 32→Asp; Leu 33→Glu; Glu 34→Val; Leu 56→Asp; Ser 58→Phe; Arg 60→Phe; Cys 61→Trp; Cys 101→Ser; Leu 105→Cys; His 106→Ala; Lys 108→Phe; Arg 111→Pro; Lys 114→Trp; and Cys 153→Ser;
(k) Ser 14→Pro; Asp 25→Ser; Arg 26→Gly; Phe 28→Asp; Met 31→Leu; Asn 32→Trp; Lys 52→Ser; Met 55→Ala; Ser 58→Asp; Ala 66→Asn; Ala 79→Glu; Ala 86→Asp; Cys 101→Phe; Leu 105→Gly; His 106→Tyr; Lys 108→Thr; Val 110→Gly; Gly 112→Met; Lys 114→Ala; and Lys 121→Thr;
(l) Ser 14→Pro; Asp 25→Ser; Arg 26→Ala; Phe 28→Asp; Met 31→Leu; Asn 32→Val; Lys 52→Ser; Met 55→Ala; Ser 58→Asp; Ala 66→Asn; Ala 79→Glu; Ala 86→Asp; Cys 101→Phe; Leu 105→Gly; Lys 108→Thr; Val 110→Ala; Gly 112→Thr; Lys 114→Ala; and Lys 121→Thr;
(m) Ser 14→Pro; Asp 25→Ser; Arg 26→Asp; Phe 28→Asp; Asn 32→Thr; Lys 52→Ser; Met 55→Ala; Ser 58→Asp; Ala 66→Asn; Ala 79→Glu; Ala 86→Asp; Cys 101→Phe; Leu 105→Gly; His 106→Gln; Lys 108→Thr; Val 110→Gly; Gly 112→Met; Lys 114→Ala; and Lys 121→Thr;
(n) Ser 14→Pro; Asp 25→Ser; Arg 26→Glu; Phe 28→Asp; Asn 32→Thr; Lys 52→Ser; Met 55→Ala; Ser 58→Asp; Ala 66→Asn; Ala 79→Glu; Ala 86→Asp; Cys 101→Phe; Leu 105→Gly; Lys 108→Thr; Val 110→Gly; Gly 112→Met; Lys 114→Ala; and Lys 121→Thr;
(o) Ser 14→Pro; Asp 25→Ser; Arg 26→Gly; Phe 28→Asp; Asn 32→Met; Lys 52→Arg; Met 55→Val; Ser 58→Asp; Ala 66→Asn; Ala 79→Glu; Ala 86→Asp; Cys 101→Phe; Leu 105→Gly; His 106→Gln; Lys 108→Thr; Val 110→Gly; Gly 112→Met; Lys 114→Ala; and Lys 121→Thr; or
(p) Arg 26→Phe; Glu 27→Val; Phe 28→Cys; Pro 29→Leu; Glu 30→Asn; Met 31→Asp; Asn 32→Tyr; Leu 33→Pro; Leu 56→Gln; Ser 58→Trp; Arg 60→Glu; Cys 61→Pro; Cys 101→Ser; Glu 104→Tyr; Leu 105→Cys; His 106→Thr; Lys 108→Tyr; Arg 111→Pro; Lys 114→Trp; and Cys 153→Ser.

23. The fusion polypeptide of claim 1, wherein the second subunit is a LAG-3-specific lipocalin mutein comprising an amino acid sequence selected from the group consisting of SEQ ID NOs: 13-28 or of a fragment or variant thereof.

24. The fusion polypeptide of claim 1, wherein the second subunit is a LAG-3-specific lipocalin mutein having at least 85% sequence identity to an amino acid sequence selected from the group consisting of SEQ ID NOs: 13-28.

25. The fusion polypeptide of claim 1, wherein one subunit can be linked to another subunit via a linker.

26. The fusion polypeptide of claim 25, wherein the linker is an unstructured (Gly-Gly-Gly-Gly-Ser)₃ linker (SEQ ID NO: 2).

27. The fusion polypeptide of claim 1, wherein the first subunit is a monoclonal antibody.

28. The fusion polypeptide of claim 27, wherein the variable region of the heavy chain of the monoclonal antibody is selected from a group consisting of SEQ ID NOs: 59-84 and 112-117 and wherein the variable region of the light chain of the monoclonal antibody is selected from a group consisting of SEQ ID NOs: 85-111 and 118-123.

29. The fusion polypeptide of claim 27, wherein the variable region of the heavy chain of the monoclonal antibody has at least 85% sequence identity to an amino acid sequence selected from the group consisting of SEQ ID NOs: 59-84 and 112-117 and wherein the variable region of the light chain of the monoclonal antibody has at least 85% sequence identity to an amino acid sequence selected from the group consisting of SEQ ID NOs: 85-111 and 118-123.

30. The fusion polypeptide of claim 27, wherein the monoclonal antibody has one of the following sets of CDR sequences:
a. VH-CDR1: GYTFTDYE (SEQ ID NO: 163), VH-CDR2: IDPGTGGT (SEQ ID NO: 164), VH-CDR3: TSEKFGSNYYFDY (SEQ ID NO: 165), VL-CDR1: QTIVHSDGNTY (SEQ ID NO: 166), VL-CDR2: KVS, VL-CDR3: FQGSHVPLT (SEQ ID NO: 167); or
b. VH-CDR1: GYTFTSYW (SEQ ID NO: 168), VH-CDR2: IDPSNSET (SEQ ID NO: 169), VH-CDR3: ARSRGNYAYEMDY (SEQ ID NO: 170), VL-CDR1: SSVSSNY (SEQ ID NO: 171), VL-CDR2: STS, VL-CDR3: HQWSSYPP (SEQ ID NO: 172); or
c. VH-CDR1: GYTFTDYW (SEQ ID NO: 173), VH-CDR2: IDTSDSYT (SEQ ID NO: 174), VH-CDR3: ARRDYGGFGY (SEQ ID NO: 175), VL-CDR1: QDISSY (SEQ ID NO: 176), VL-CDR2: YTS, VL-CDR3: QQYSELPW (SEQ ID NO: 177); or
d. VH-CDR1: GYTFTDYN (SEQ ID NO: 178), VH-CDR2: IDPNNGDT (SEQ ID NO: 179), VH-CDR3: ARWRSSMDY (SEQ ID NO: 180), VL-CDR1: QGISNY (SEQ ID NO: 181), VL-CDR2: YTS, VL-CDR3: QQYSNLPW (SEQ ID NO: 182); or
e. VH-CDR1: GYSITSDYA (SEQ ID NO: 183), VH-CDR2: ITYSGSP (SEQ ID NO: 184), VH-CDR3: ARGLGGHYFDY (SEQ ID NO: 185), VL-CDR1: QSISDY (SEQ ID NO: 186), VL-CDR2: YAS, VL-CDR3: QNGRSYPY (SEQ ID NO: 187); or
f. VH-CDR1: GFSLTSYG (SEQ ID NO: 188), VH-CDR2: IWRGGNT (SEQ ID NO: 189), VH-CDR3: AASMIGGY (SEQ ID NO: 190), VL-CDR1: QSIVHSNGNTY (SEQ ID NO: 191), VL-CDR2: KVS, VL-CDR3: FQGSHVPL (SEQ ID NO: 192).

31. The fusion polypeptide of claim 27, wherein the monoclonal antibody is selected from the group consisting of nivolumab, pembrolizumab, PDR001, MEDI0680, pidilizumab, ENUM-388D4, and ENUM-244C8.

32. The fusion polypeptide of claim 27, wherein the monoclonal antibody has an IgG4 backbone.

33. The fusion polypeptide of claim 32 wherein the IgG4 backbone has any one or more of the following mutations selected from the group consisting of S228P, N297A, F234A, and L235A.

34. The fusion polypeptide of claim 27, wherein the monoclonal antibody has an IgG1 backbone.

35. The fusion polypeptide of claim 34, wherein the IgG1 backbone has any one or more of the following mutations selected from the group consisting of N297A, L234A and L235A.

36. The fusion polypeptide of claim 1, wherein the fusion polypeptide comprises the amino acids shown in SEQ ID NOs: 4 and 5, or the amino acids shown in SEQ ID NOs: 4 and 9, or the amino acids shown in SEQ ID NOs: 4 and 6, or the amino acids shown in SEQ ID NOs: 4 and 10, or the amino acids shown in SEQ ID NOs: 3 and 7, or the amino acids shown in SEQ ID NOs: 3 and 8, or the amino acids shown in SEQ ID NOs: 3 and 11, or the amino acids shown in SEQ ID NOs: 3 and 12.

37. A nucleic acid molecule comprising a nucleotide sequence encoding the polypeptide of claim 1.

38-39. (canceled)

40. A host cell containing a nucleic acid molecule of claim 37.

41. A method of producing the fusion polypeptide of claim 1, wherein the fusion polypeptide is produced from the nucleic acid coding for the fusion polypeptide.

42.-44. (canceled)

45. A method of simultaneously inhibiting immune checkpoints PD-1 and LAG-3 in a subject, comprising administering to the subject one or more fusion polypeptides of claim 1 or a composition comprising such fusion polypeptides.

46. A method of increasing anti-tumor lymphocyte cell activity in a subject, comprising administering to the subject one or more fusion polypeptides of claim 1 or a composition comprising such fusion polypeptides.

47. A method of interfering with the binding of human LAG-3 to major histocompatibility complex (MHC) class II in a subject, comprising administering to the subject one or more fusion polypeptides of claim 1 or one or more compositions comprising such fusion polypeptides.

48. The fusion polypeptide of claim 1, wherein the fusion polypeptide is capable of inducing lymphocyte cell proliferation.

49. A pharmaceutical composition comprising a fusion polypeptide of claim 1 and a pharmaceutically acceptable excipient.

50. A method of preventing, ameliorating, or treating cancers in a subject, comprising administering to the subject the fusion polypeptide of claim 1 or a composition comprising such fusion polypeptide.


SEQUENCE LISTING (192 sequences)

SEQ ID NO: 1 - protein, 158 aa, Homo sapiens
  Human tear lipocalin
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 2 - protein, 15 aa, Artificial Sequence
  (G4S)3 linker
GGGGSGGGGSGGGGS

SEQ ID NO: 3 - protein, 439 aa, Artificial Sequence
  heavy chain of PD-1 benchmark antibody 1
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 214 aa, Artificial Sequence
  light chain of PD-1 benchmark antibody 1
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 606 aa, Artificial Sequence
  bispecific fusion polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 6 - protein, 608 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGSDGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 381 aa, Artificial Sequence
  bispecific fusion polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 8 - protein, 383 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGSDGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 606 aa, Artificial Sequence
  bispecific fusion polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 10 - protein, 608 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGSDGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 381 aa, Artificial Sequence
  bispecific fusion polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 12 - protein, 383 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGSDGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 152 aa, Artificial Sequence
  lipocalin mutein
ASDEEIQDVSGTWYLKAMTVDSDCFWIEEWSVTPMTLTTLEGGNLEAKVTMDIFGFWQEVKAVLEKTDEPGKYTADGGKHVAYIIRSHVKDHYIFYSEGECAGFPVPGVWLVGRDPKNNLEALEDFEKAAGARGLSTESILIPRQSETSSPG

SEQ ID NO: 14 - protein, 152 aa, Artificial Sequence
  lipocalin mutein
ASDEEIQDVPGTWYLKAMTVSGEDPEMTLESVTPMTLTTLEGGNLEANVTALIDGRCQEVKNVLEKTDEPGKYTEDGGKHVDYIIRSHVKDHYIFYFEGEGHGTPARTVALVGRDPTNNLEALEDFEKAAGARGLSTESILIPRQSETCSPG

SEQ ID NO: 15 - protein, 152 aa, Artificial Sequence
  lipocalin mutein
ASDEEIQDVSGTWYLKAMTVDFVCLYDVPESVTPMTLTTLEGGNLEAKVTMQIWGELQEVKAVLEKTDEPGKYTADGGKHVAYIIRSHVKDHYIFYSEGYCVGYPVPGVWLVGRDPKNNLEALEDFEKAAGARGLSTESILIPRQSETSSPG

SEQ ID NO: 16 - protein, 152 aa, Artificial Sequence
  lipocalin mutein
ASDEEIQDVSGTWYLKAMTVDETCWWYVDHSVTPMTLTTLEGGNLEAKVTMNLWGFWQEVKAVLEKTDEPGKYTADGGKHVAYIIRSHVKDHYIFYSEGECQGWPVPGVWLVGRDPKNNLEALEDFEKAAGARGLSTESILIPRQSETSSPG

SEQ ID NO: 17 - protein, 152 aa, Artificial Sequence
  lipocalin mutein
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 18 - protein, 152 aa, Artificial Sequence; identical to SEQ ID NO: 17
  lipocalin mutein

SEQ ID NO: 19 - protein, 152 aa, Artificial Sequence
  lipocalin mutein
ASDEEIQDVSGTWYLKAMTVDSDCFWIEEWSATPMTLTTLEGGDLEAKVTMDIFGFWQEVKAVLEKTDEPGKYTADGGKHAAYIIRSHVKDHYIFYSEGECAGFPVPGVWLVGRDPKNNLEALEDFEKAAGARGLGTESILIPRQSETSSPG

SEQ ID NO: 20 - protein, 152 aa, Artificial Sequence
  lipocalin mutein
ASDEEIQDVSGTWYLKAMTVDSDCFWIDEVSVTPMTLTTLEGGNLEAKVTMDIFGFWQEVKAVLEKTDEPGKYTADGGKHVAYIIRSHVKDHYIFYSEGECEGFPVPGVWLVGRDPKNNLEALEDFEKAAGARGLSTESILIPRQSETSSPG

SEQ ID NO: 21 - protein, 152 aa, Artificial Sequence
  lipocalin mutein
ASDEEIQDVSGTWYLKAMTVDSDCFWIEEWSATPMTLTTLEGGNLEAEATMDIFGFWQEVKAVLEKTDEPGKYTADGGKHVAYIIRSHVKDHYIFYSEGECAGFPVPGVWLVGRDPKNNLEALEDFEKAAGARGLSTESILIPRQSETSSPG

SEQ ID NO: 22 - protein, 152 aa, Artificial Sequence
  lipocalin mutein
ASDEEIQDVSGTWYLKAMTVDSDCFWVDEVSVTPMTLTTLEGGNLEAKVTMDIFGFWQEVKAVLEKTDEPGKYTADGGKHVAYIIRSHVKDHYIFYSEGECAGFPVPGVWLVGRDPKNNLEALEDFEKAAGARGLSTESILIPRQSETSSPG

SEQ ID NO: 23 - protein, 152 aa, Artificial Sequence
  lipocalin mutein
ASDEEIQDVPGTWYLKAMTVSGEDPELWLESVTPMTLTTLEGGNLEASVTALIDGRCQEVKNVLEKTDEPGKYTEDGGKHVDYIIRSHVKDHYIFYFEGEGYGTPGRMVALVGRDPTNNLEALEDFEKAAGARGLSTESILIPRQSETCSPG

SEQ ID NO: 24 - protein, 152 aa, Artificial Sequence
  lipocalin mutein
ASDEEIQDVPGTWYLKAMTVSAEDPELVLESVTPMTLTTLEGGNLEASVTALIDGRCQEVKNVLEKTDEPGKYTEDGGKHVDYIIRSHVKDHYIFYFEGEGHGTPARTVALVGRDPTNNLEALEDFEKAAGARGLSTESILIPRQSETCSPG

SEQ ID NO: 25 - protein, 152 aa, Artificial Sequence
  lipocalin mutein
ASDEEIQDVPGTWYLKAMTVSDEDPEMTLESVTPMTLTTLEGGNLEASVTALIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 26 - protein, 152 aa, Artificial Sequence
  lipocalin mutein
ASDEEIQDVPGTWYLKAMTVSEEDPEMTLESVTPMTLTTLEGGNLEASVTALIDGRCQEVKNVLEKTDEPGKYTEDGGKHVDYIIRSHVKDHYIFYFEGEGHGTPGRMVALVGRDPTNNLEALEDFEKAAGARGLSTESILIPRQSETCSPG

SEQ ID NO: 27 - protein, 152 aa, Artificial Sequence
  lipocalin mutein
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 28 - protein, 152 aa, Artificial Sequence
  lipocalin mutein
ASDEEIQDVSGTWYLKAMTVDFVCLNDYPESVTPMTLTTLEGGNLEAKVTMQIWGEPQEVKAVLEKTDEPGKYTADGGKHVAYIIRSHVKDHYIFYSEGYCTGYPVPGVWLVGRDPKNNLEALEDFEKAAGARGLSTESILIPRQSETSSPG

SEQ ID NO: 29 - DNA, 1818 bp, Artificial Sequence
  bispecific fusion polypeptide
caggtgcagctggtggaatctggcggcggagtggtgcagcctggcagatctctgagactggactgcaaggcctccggcatcaccttctccaactccggcatgcactgggtgcgacaggcccctggaaagggcctggaatgggtggccgtgatttggtacgacggctccaagcggtactacgccgactctgtgaagggccggttcaccatctcccgggacaactccaagaacaccctgtttctgcagatgaactccctgcgggccgaggacaccgccgtgtactactgtgccaccaacgacgactactggggccagggcacactcgtgaccgtgtcctctgcttccaccaagggcccctccgtgtttcctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctgggaggtggcggaggatctggcggaggcggttctggcggcggtggatctgcttccgacgaagagatccaggacgtgtccggcacctggtatctgaaggccatgaccgtggactccgactgcttctggatcgacgatgtgtccgtgacccccatgaccctgaccaccctggaaggcggcaacctggaagccaaagtgacaatggatatcttcggcttctggcaggaagtgaaggccgtgctggaaaagaccgacgagcccggcaagtacaccgccgatggcggcaagcacgtggcctacatcatccggtcccacgtgaaggaccactacatcttctactccgagggcgagtgcgccggctatcctgtgcctggcgtgtggctcgtgggccgggatcctaagaacaatctggaagccctggaagatttcgagaaggccgctggcgctcggggcctgtctacagagtctatcctgatcccccggcagtccgagacatcctcccctggc

SEQ ID NO: 30 - DNA, 1824 bp, Artificial Sequence
  bispecific fusion polypeptide
gcctctgacgaagagatccaggacgtgtccggcacctggtatctgaaggccatgaccgtggactccgactgcttctggatcgacgatgtgtccgtgacccccatgaccctgaccaccctggaaggcggcaacctggaagccaaagtgacaatggatatcttcggcttctggcaggaagtgaaggccgtgctggaaaagaccgacgagcccggcaagtacaccgccgatggcggcaagcacgtggcctacatcatccggtcccacgtgaaggaccactacatcttctactccgagggcgagtgcgccggctatcctgtgcctggcgtgtggctcgtgggccgggatcctaagaacaatctggaagccctggaagatttcgagaaggccgctggcgctcggggcctgtctacagagtctatcctgatcccccggcagtccgagacatcctcccctggatctgatggcggcggaggatctggcggaggtggaagcggagggggaggatctcaggtgcagctggtggaatctggcggcggagtggtgcagcctggcagatctctgagactggactgcaaggcctccggcatcaccttctccaactccggcatgcactgggtgcgacaggcccctggaaagggcctggaatgggtggccgtgatttggtacgacggctccaagcggtactacgccgactctgtgaagggccggttcaccatctcccgggacaactccaagaacaccctgtttctgcagatgaactccctgcgggccgaggacaccgccgtgtactactgtgccaccaacgacgactactggggccagggcacactcgtgaccgtgtcctctgcttccaccaagggcccctccgtgtttcctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctggga

SEQ ID NO: 31 - DNA, 1143 bp, Artificial Sequence
  bisecific fusion polypeptide
gagatcgtgctgacccagtctcctgccaccctgtctctgagccctggcgagagagctaccctgtcctgcagagcctcccagtccgtgtcctcttacctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagtcctccaactggccccggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgcggaggcggaggatctggtggtggtggatctggcggcggaggctctgcctctgacgaagagatccaggacgtgtccggcacctggtatctgaaggccatgaccgtggactccgactgcttctggatcgacgatgtgtccgtgacccccatgaccctgaccaccctggaaggcggcaacctggaagccaaagtgacaatggatatcttcggcttctggcaggaagtgaaggccgtgctggaaaagaccgacgagcccggcaagtacaccgccgatggcggcaagcacgtggcctacatcatccggtcccacgtgaaggaccactacatcttctactccgagggcgagtgtgccggctaccctgtgcctggcgtgtggctcgtgggcagggaccctaagaacaatctggaagccctggaagatttcgagaaggccgctggcgctcggggcctgtctacagagtctatcctgatcccccggcagtccgagacatcctcccctggc

SEQ ID NO: 32 - DNA, 1149 bp, Artificial Sequence
  bispecific fusion polypeptide
gcctctgacgaagagatccaggacgtgtccggcacctggtatctgaaggccatgaccgtggactccgactgcttctggatcgacgatgtgtccgtgacccccatgaccctgaccaccctggaaggcggcaacctggaagccaaagtgacaatggatatcttcggcttctggcaggaagtgaaggccgtgctggaaaagaccgacgagcccggcaagtacaccgccgatggcggcaagcacgtggcctacatcatccggtcccacgtgaaggaccactacatcttctactccgagggcgagtgcgccggctatcctgtgcctggcgtgtggctcgtgggccgggatcctaagaacaatctggaagccctggaagatttcgagaaggccgctggcgctcggggcctgtctacagagtctatcctgatcccccggcagtccgagacatcctcccctggatctgatggcggcggaggatctggcggaggtggaagcggagggggaggatctgagatcgtgctgacccagtctcctgccaccctgtctctgagccctggcgagagagctaccctgtcctgcagagcctcccagtccgtgtcctcttacctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagtcctccaactggccccggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc

SEQ ID NO: 33 - DNA, 1818 bp, Artificial Sequence
  bispecific fusion polypeptide
caggtgcagctggtggaatctggcggcggagtggtgcagcctggcagatctctgagactggactgcaaggcctccggcatcaccttctccaactccggcatgcactgggtgcgacaggcccctggaaagggcctggaatgggtggccgtgatttggtacgacggctccaagcggtactacgccgactctgtgaagggccggttcaccatctcccgggacaactccaagaacaccctgtttctgcagatgaactccctgcgggccgaggacaccgccgtgtactactgtgccaccaacgacgactactggggccagggcacactcgtgaccgtgtcctctgcttccaccaagggcccctccgtgtttcctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctgggaggtggcggaggatctggcggaggcggttctggcggcggtggatctgcttccgacgaagagatccaggacgtgcccggcacctggtatctgaaggccatgaccgtgtccggcgaggaccccgagatgatgctggaatccgtgacccccatgaccctgaccaccctggaaggcggcaacctggaagccagagtgaccgtgctgatcgacggccggtgccaggaagtgaagaacgtgctggaaaagaccgacgagcccggcaagtacaccgaggatggcggaaagcacgtggactacatcatccggtcccacgtgaaggaccactacatcttctacttcgagggcgagggccagggcacccctggaagaatggtggctctcgtgggccgggaccccaccaacaatctggaagctctggaagatttcgagaaggccgctggcgctcggggcctgtctacagagtctatcctgatcccccggcagtccgagacatgctcccctggc

SEQ ID NO: 34 - DNA, 1824 bp, Artificial Sequence
  bispecific fusion polypeptide
gcctctgacgaagagatccaggatgtgcccggcacctggtatctgaaggccatgaccgtgtccggcgaggaccccgagatgatgctggaatccgtgacccccatgaccctgaccaccctggaaggcggcaacctggaagccagagtgaccgtgctgatcgacggccggtgccaggaagtgaagaacgtgctggaaaagaccgacgagcccggcaagtacaccgaggatggcggaaagcacgtggactacatcatccggtcccacgtgaaggaccactacatcttctacttcgagggcgagggccagggcacccctggaagaatggtggctctcgtgggccgggaccccaccaacaatctggaagctctggaagatttcgagaaggccgctggcgctcggggcctgtctacagagtctatcctgatcccccggcagtccgagacatgctcccctggatctgatggcggcggaggatctggcggaggtggaagcggagggggaggatctcaggtgcagctggtggaatctggcggcggagtggtgcagcctggcagatctctgagactggactgcaaggcctccggcatcaccttctccaactccggcatgcactgggtgcgacaggcccctggaaagggcctggaatgggtggccgtgatttggtacgacggctccaagcggtactacgccgactctgtgaagggccggttcaccatctcccgggacaactccaagaacaccctgtttctgcagatgaactccctgcgggccgaggacaccgccgtgtactactgtgccaccaacgacgactactggggccagggcacactcgtgaccgtgtcctctgcttccaccaagggcccctccgtgtttcctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctggga

SEQ ID NO: 35 - DNA, 1143 bp, Artificial Sequence
  bispecific fusion polypeptide
gagatcgtgctgacccagtctcctgccaccctgtctctgagccctggcgagagagctaccctgtcctgcagagcctcccagtccgtgtcctcttacctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagtcctccaactggccccggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgcggaggcggaggatctggtggtggtggatctggcggcggaggctctgcctctgacgaagagatccaggatgtgcccggcacctggtatctgaaggccatgaccgtgtccggcgaggaccccgagatgatgctggaatccgtgacccccatgaccctgaccaccctggaaggcggcaacctggaagccagagtgaccgtgctgatcgacggccggtgccaggaagtgaagaacgtgctggaaaagaccgacgagcccggcaagtacaccgaggatggcggaaagcacgtggactacatcatccggtcccacgtgaaggaccactacatcttctacttcgagggcgagggccagggcacccctggaagaatggtggctctcgtgggccgggaccccaccaacaatctggaagctctggaagatttcgagaaggccgctggcgctcggggcctgtctacagagtctatcctgatcccccggcagtccgagacatgctcccctggc

SEQ ID NO: 36 - DNA, 1149 bp, Artificial Sequence
  bispecific fusion polypeptide
gcctctgacgaagagatccaggatgtgcccggcacctggtatctgaaggccatgaccgtgtccggcgaggaccccgagatgatgctggaatccgtgacccccatgaccctgaccaccctggaaggcggcaacctggaagccagagtgaccgtgctgatcgacggccggtgccaggaagtgaagaacgtgctggaaaagaccgacgagcccggcaagtacaccgaggatggcggaaagcacgtggactacatcatccggtcccacgtgaaggaccactacatcttctacttcgagggcgagggccagggcacccctggaagaatggtggctctcgtgggccgggaccccaccaacaatctggaagctctggaagatttcgagaaggccgctggcgctcggggcctgtctacagagtctatcctgatcccccggcagtccgagacatgctcccctggatctgatggcggcggaggatctggcggaggtggaagcggagggggaggatctgagatcgtgctgacccagtctcctgccaccctgtctctgagccctggcgagagagctaccctgtcctgcagagcctcccagtccgtgtcctcttacctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagtcctccaactggccccggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc

SEQ ID NO: 37 - DNA, 456 bp, artificial
  lipocalin mutein encoding the lipocalin mutein of SEQ ID NO 17
gcctcagacgaggagattcaggatgtgtcagggacgtggtatctgaaggccatgacggtggattctgattgcttttggattgatgatgtgtcagttacgccaatgactctgactacccttgaaggcggcaatctggaggctaaggtcaccatggatatttttggcttttggcaggaagtgaaagcagtgttagagaagacagatgaaccgggtaaatatacggccgatggcggcaaacatgttgcctatatcattcgcagccatgtgaaagatcattacatcttttatagcgagggcgaatgcgctggctatccggttccaggggtgtggctcgtgggcagagaccccaagaacaacctggaagccttggaggactttgagaaagccgcaggagcccgcggactcagcacggagagcatcctcatccccaggcagagcgaaaccagctctccaggg

SEQ ID NO: 38 - DNA, 456 bp, artificial
  lipocalin mutein encoding the lipocalin mutein of SEQ ID NO 27
gcctcagacgaggagattcaggatgtgccagggacgtggtatctgaaagcgatgacggtttcgggggaagatcctgagatgatgctggaatcagttacgccaatgactctgactacccttgaaggcggcaatctggaggctcgtgtgaccgttctgattgatggccgctgccaggaagtgaaaaatgtgctcgagaagacagatgaaccgggtaaatacacggaggatggcggcaaacatgtggattatatcattagatctcatgtgaaagatcattacatcttctactttgaaggcgaagggcagggcacgccgggtcgcatggtggctctggtgggcagagaccccaccaataatctggaagccttggaggactttgagaaagccgcaggagcccgcggactcagcacggagagcatcctcatccccaggcagagcgaaacctgctctccaggg

SEQ ID NO: 39 - DNA, 1317 bp, artificial
  heavy chain of benchmark antibody 1 (SEQ ID NO 3)
caggtgcagctggtggaatctggcggcggagtggtgcagcctggcagatctctgagactggactgcaaggcctccggcatcaccttctccaactccggcatgcactgggtgcgacaggcccctggaaagggcctggaatgggtggccgtgatttggtacgacggctccaagcggtactacgccgactctgtgaagggccggttcaccatctcccgggacaactccaagaacaccctgtttctgcagatgaactccctgcgggccgaggacaccgccgtgtactactgtgccaccaacgacgactactggggccagggcacactcgtgaccgtgtcctctgcttccaccaagggcccctccgtgtttcctctggccccttgctccagatccacctccgagtctaccgccgctctgggctgcctcgtgaaggactacttccccgagcccgtgacagtgtcttggaactctggcgccctgacctccggcgtgcacacctttccagctgtgctgcagtcctccggcctgtactccctgtcctccgtcgtgactgtgccctccagctctctgggcaccaagacctacacctgtaacgtggaccacaagccctccaacaccaaggtggacaagagagtggaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctggga

SEQ ID NO: 40 - DNA, 642 bp, artificial
  light chain of benchmark antibody 1 (SEQ ID NO 4)
gagatcgtgctgacccagtctcctgccaccctgtctctgagccctggcgagagagctaccctgtcctgcagagcctcccagtccgtgtcctcttacctggcctggtatcagcagaagcccggccaggctccccggctgctgatctacgatgcctccaatagagccaccggcatccctgccagattctccggctctggctctggcaccgactttaccctgaccatctccagcctggaacccgaggacttcgccgtgtactactgccagcagtcctccaactggccccggacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc

SEQ ID NO: 41 - protein, 395 aa, artificial
  Fc lipocalin mutein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 42 - protein, 395 aa, artificial
  Fc lipocalin mutein
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 43 - protein, 152 aa, artificial
  lipocalin mutein negative control
ASDEEIQDVSGTWYLKAMTVDRECPEMNLESVTPMTLTTLEGGNLEAKVTMLISGRSQEVKAVLEKTDEPGKYTADGGKHVAYIIRSHVKDHYIFYSEGECHGKPVPGVWLVGRDPKNNLEALEDFEKAAGARGLSTESILIPRQSETSSPG

SEQ ID NO: 44 - DNA, 1185 bp, artificial
  Fc lipocalin mutein encoding SEQ ID NO 41
gaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctgggaggtggcggaggatctggcggaggcggttctggcggcggtggatctgcttccgacgaagagatccaggacgtgtccggcacctggtatctgaaggccatgaccgtggactccgactgcttctggatcgacgatgtgtccgtgacccccatgaccctgaccaccctggaaggcggcaacctggaagccaaagtgacaatggatatcttcggcttctggcaggaagtgaaggccgtgctggaaaagaccgacgagcccggcaagtacaccgccgatggcggcaagcacgtggcctacatcatccggtcccacgtgaaggaccactacatcttctactccgagggcgagtgcgccggctatcctgtgcctggcgtgtggctcgtgggccgggatcctaagaacaatctggaagccctggaagatttcgagaaggccgctggcgctcggggcctgtctacagagtctatcctgatcccccggcagtccgagacatcctcccctggc

SEQ ID NO: 45 - DNA, 1185 bp, artificial
  Fc lipocalin mutein encoding SEQ ID NO 42
gaatctaagtacggccctccctgccccccttgtcctgcccctgaatttctgggcggaccttccgtgttcctgttccccccaaagcctaaggacaccctgatgatctcccggacccccgaagtgacctgcgtggtggtggatgtgtcccaggaagatcccgaggtgcagttcaattggtacgtggacggcgtggaagtgcacaacgccaagaccaagcctagagaggaacagttcaactccacctaccgggtggtgtccgtgctgaccgtgctgcaccaggattggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactataagaccaccccccctgtgctggactccgacggcagcttcttcctgtactctcgcctgaccgtggacaagtcccggtggcaggaaggcaacgtgttctcctgctccgtgatgcacgaggccctgcacaaccactacacccagaagtccctgtccctgtctctgggaggtggcggaggatctggcggaggcggttctggcggcggtggatctgcttccgacgaagagatccaggacgtgcccggcacctggtatctgaaggccatgaccgtgtccggcgaggaccccgagatgatgctggaatccgtgacccccatgaccctgaccaccctggaaggcggcaacctggaagccagagtgaccgtgctgatcgacggccggtgccaggaagtgaagaacgtgctggaaaagaccgacgagcccggcaagtacaccgaggatggcggaaagcacgtggactacatcatccggtcccacgtgaaggaccactacatcttctacttcgagggcgagggccagggcacccctggaagaatggtggctctcgtgggccgggaccccaccaacaatctggaagctctggaagatttcgagaaggccgctggcgctcggggcctgtctacagagtctatcctgatcccccggcagtccgagacatgctcccctggc

SEQ ID NO: 46 - DNA, 456 bp, artificial
  lipocalin mutein control encoding SEQ ID NO 43
gcctcagacgaggagattcaggatgtgtcagggacgtggtatctgaaggccatgacggtggacagggagtgccctgagatgaatctggaatcggtgacacccatgaccctcacgaccctggaagggggcaacctggaagccaaggtcaccatgctgataagtggccggagccaggaggtgaaggccgtcctggagaaaactgacgagccgggaaaatacacggccgacgggggcaagcacgtggcatacatcatcaggtcgcacgtgaaggaccactacatcttttactctgagggcgagtgccacgggaagccggtcccaggggtgtggctcgtgggcagagaccccaagaacaacctggaagccttggaggactttgagaaagccgcaggagcccgcggactcagcacggagagcatcctcatccccaggcagagcgaaaccagctctccaggg

SEQ ID NO: 47 - protein, 447 aa, artificial
  heavy chain of PD-1 benchmark antibody 2
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 218 aa, artificial
  light chain of PD-1 benchmark antibody 2
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 447 aa, Artificial Sequence
  heavy chain of LAG-3 benchmark antibody
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  light chain of LAG-3 benchmark antibody
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 614 aa, Artificial Sequence
  bispecific fusion polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 52 - protein, 614 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 385 aa, Artificial Sequence
  bispecific fusion polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 54 - protein, 385 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 614 aa, Artificial Sequence
  bispecific fusion polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 56 - protein, 614 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 385 aa, Artificial Sequence
  bispecific fusion polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 58 - protein, 385 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 120 aa, Mus musculus
  heavy chain variable region of PD-1 antibody
EVQLQESGPELVRPGASVKMSCKASGYTFTSYWMHWVKQRPGQGLEWIGMIDPSNSETSLNQKFKDKATLNVDKSTNTAYMQLSSLTSEDSAVYYCARSRGNYAYEMDYWGQGTSVTVSS

SEQ ID NO: 60 - protein, 120 aa, Mus musculus
  heavy chain variable region of PD-1 antibody
EVQLQESGPELVRPGASVKMSCKASGYTFTSYWMHWVKQRPGQGLEWIGMIEPSSSETSLNQKFKDKATLNVDKSSNTAYMQLSSLTSEDSAVYYCARSRGNYAYEMDYWGQGTSVTVSS

SEQ ID NO: 61 - protein, 120 aa, Mus musculus
  heavy chain variable region of PD-1 antibody
EVQLQESGPELVRPGASVKMSCKASGYTFTSYWMHWVKQRPGQGLEWIGMIDPYSSETSLNQKFKDKATLNVDKISNTAYMQLSSLTSEDSAVYFCARSRGNYAYDMDYWGQGTSVTVSS

SEQ ID NO: 62 - protein, 120 aa, Mus musculus
  heavy chain variable region of PD-1 antibody
EVQLQESGPELVRPGASVKMSCKASGYTFTSYWMHWVKQRPGQGLEWIGMIDPSNSETSLNQKFKDKATLNVDKSSKTAYMQLSSLTSEDSAVYYCARSRGNYAYDMDYWGQGTSVTVSS

SEQ ID NO: 63 - protein, 117 aa, Mus musculus
  heavy chain variable region of PD-1 antibody
EVQLQESGAELVMPGASVKMSCKASGYTFTDYWMHWVKQRPGQGLEWIGAIDTSDSYTSYHQNFKGKATLTEDESSSTAYMQLSSLTSEDSAIYYCARRDYGGFGYWGQGTTLTVSS

SEQ ID NO: 64 - protein, 116 aa, Mus musculus
  heavy chain variable region of PD-1 antibody
EVQLQESGPELVKPGASVKIPCKASGYTFTDYNMDWVKKSHGKSLEWIGDIDPNNGGTIYNQKFKGKATLTVDKSSRTAYMELRSLTSEDTAVYYCARWRSSMDYWGQGTSVTVSS

SEQ ID NO: 65 - protein, 116 aa, Mus musculus
  heavy chain variable region of PD-1 antibody
EVQLQESGPELVKPGASVKIPCRASGYIFTDYNMDWVKQSHGKSLEWIGDIDPNNGGTIYNQKFKDKTTLTVDKSSRTAYMELRSLTSEDTAVYYCARWRSSMDYWGQGTSVTVSS

SEQ ID NO: 66 - protein, 116 aa, Mus musculus
  heavy chain variable region of PD-1 antibody
EVQLQESGPELVKPGASVKIPCKASGYTFTDYNMDWVKQNHGKSLEWIGDIDPNNGDTIYNQKFKGKATLTVDKSSRTAYMELRSLTSEDTAVYYCARWRSSMDYWGQGTSVTVSS

SEQ ID NO: 67 - protein, 116 aa, Mus musculus
  heavy chain variable region of PD-1 antibody
EVQLQESGPELVKPGASVKIPCKASGYTFTDYNMDWVKQSHGKSLEWIGDIDPNSGGSIYNQKFKGKATLTVDKSSRTVYMELRSLTSEDTAVYYCARWRSSMDYWGQGTSVTVSS

SEQ ID NO: 68 - protein, 116 aa, Mus musculus
  heavy chain variable region of PD-1 antibody
EVQLQESGPELVKPGASVKITCKASGYTFTDYNMDWVKQSHGKSLEWIGDIDPNNGGTIYNQKFKGKATLTVDKSSNTAYMELRSLASEDTAVYYCARWRSSMDYWGQGTSVSVSS

SEQ ID NO: 69 - protein, 116 aa, Mus musculus
  heavy chain variable region of PD-1 antibody
EVQLQESGPELVKPGASVKIPCKASGYTFTDYNMDWVKQSHGKSLEWIGDIDPNNGGTIYNQNFKGKATLTVDKSSSTAYMELRSLTSEDTAVYYCARWRSSMDYWGQGTSVTVSS

SEQ ID NO: 70 - protein, 116 aa, Mus musculus
  heavy chain variable region of PD-1 antibody
EVQLQESGPELVKPGASVKIPCKASGYTFTDYNMDWVKQSHGKSLEWIGDIDPNNGGIIYNQKFKGKAALTVDKSSSTAYMELRSLTSEDTAVYYCARWRSSMDYWGQGTSVTVSS

SEQ ID NO: 71 - protein, 116 aa, Mus musculus
  heavy chain variable region of PD-1 antibody
EVQLQESGPELVKPGASVKIPCKASGYTFTDYNMDWVKQSHGKSLEWIGDIDPNNGGIIYNQKFKGKAALTVDKSSSTAYMELRSLTSEDTAVYYCTRWRSSMDYWGQGTSVTVSS

SEQ ID NO: 72 - protein, 116 aa, Mus musculus
  heavy chain variable region of PD-1 antibody
EVQLQESGPELVKPGASVKIPCKASGYTFTDYNMDWVKQSHGKSLEWIGDIDPNNGNTIYNQKFKGKATLTVDKSSSTAYMELRSLTSEDTAVYYCTKWRSSMDYWGQGTSVTVSS

SEQ ID NO: 73 - protein, 116 aa, Mus musculus
  heavy chain variable region of PD-1 antibody
EVQLQESGPELVRPGASVKIPCKASGYTFTDYNMDWVMQSHGKSLEWIGDIDPNNGGTIYNQKFKGKATLTVDKSSSTAYMELRSLTSEDTAVYYCTRWRSSMDYWGQGTSVTVSS

SEQ ID NO: 74 - protein, 116 aa, Mus musculus
  heavy chain variable region of PD-1 antibody
EVQLQESGPELVKPGASVKIPCKASGYTFTDYNVDWVKQSHGKSLEWIGDIDPNNGGTFYNQKFKGKATLTVDKSSSTAHMELRSLTSEDTAVYYCVRWRSSMDYWGQGTSVTVSS

SEQ ID NO: 75 - protein, 116 aa, Mus musculus
  heavy chain variable region of PD-1 antibody
EVQLQESGPELVKPGASVKIPCKASGYTFTDYNMDWVKQSHGKSLEWIGDIDPNTGTTFYNQDFKGKATLTVDKSSSTAYMELRSLTSEDTAVYYCARWRSSMDYWGQGTSLTVSS

SEQ ID NO: 76 - protein, 120 aa, Mus musculus
  heavy chain variable region of PD-1 antibody
EVQLQESGAELVRPGASVTLSCKASGYTFTDYEMHWVKQTPVHGLEWIGVIDPGTGGTAYNQKFKVKALLTADKSSNTAYMELRSLTSEDSAVYYCTSEKFGSNYYFDYWGQGTTLTVSS

SEQ ID NO: 77 - protein, 120 aa, Mus musculus
  heavy chain variable region of PD-1 antibody
EVQLQESGAELVRPGASVTLSCKASGYTFTDYEIHWVKQTPVHGLEWIGAIDPETGGTAYNQKFKGKAILTADKSSSTAYMELRSLTSEDSAVYYCTSEKFGSSYYFDYWGQGTTFTVSS

SEQ ID NO: 78 - protein, 118 aa, Mus musculus
  heavy chain variable region of PD-1 antibody
EVQLQESGPGLVKPSQSLSLTCTVTGYSITSDYAWNWIRQFPGNKLEWMGYITYSGSPTYNPSLKSQFSITRDTSKNQFFLQLNSLTTEDTATYYCARGLGGHYFDYWGQGTTLTVSS

SEQ ID NO: 79 - protein, 118 aa, Mus musculus
  heavy chain variable region of PD-1 antibody
EVQLQESGPGLVKPSQSLSLTCTVTGYSITSDYAWNWIRQFPGNKLEWMGYITYSGSPTYNPSLKSQFSITRDTSKNQFFLQLNSVTTEDTATYYCARGLGGHYFDYWGQGTTLTVSS

SEQ ID NO: 80 - protein, 117 aa, Mus musculus
  heavy chain variable region of PD-1 antibody
EVQLQESGPDLVKPSQSLSLTCTVTGYSITSGYSWHWIRQFPGNKLEWMGFIHYSGDTNYNPSLKSRFSITRDTSKNQFFLHLNSVTPEDTATYYCASPSRLLFDYWGHGTTLTVSS

SEQ ID NO: 81 - protein, 113 aa, Mus musculus
  heavy chain variable region of PD-1 antibody
EVQLQESGPGLVAPSQSLSITCTVSGFSLTNYGVDWVRQSPGKGLEWLGVIWGVGSTNYNSALKSRLSISKDNSKSQVFLKMNSLQTDDTAMYYCASDGFVYWGQGTLVTVSS

SEQ ID NO: 82 - protein, 113 aa, Mus musculus
  heavy chain variable region of PD-1 antibody
EVQLQESGPGLVAPSQSLSITCTVSGFSLTSYGVDWVRQSPGKGLEWLGVIWGIGSTNYNSALKSRLSISKDNSKSQVFLKMNSLQSDDTAMYYCASDGFVYWGQGTLVTVSS

SEQ ID NO: 83 - protein, 114 aa, Mus musculus
  heavy chain variable region of PD-1 antibody
EVQLQESGPSLVQPSQSLSITCTVSGFSLTSYGVHWVRQSPGKGLEWLGVIWRGGNTDYNAAFMSRLSITKDNSKSQVFFKMNSLQADDTAIYYCAASMIGGYWGQGTTLTVSS

SEQ ID NO: 84 - protein, 114 aa, Mus musculus
  heavy chain variable region of PD-1 antibody
EVQLQESGPSLVQPSQSLSITCTVSGFSLTNYGVHWVRQSPGKGLEWLGVIWRGGNTDYNAAFMSRLSITKDNSKSQVFFKFHSLQTDDTAIYYCAASMIGGYWGQGTTLTVSS

SEQ ID NO: 85 - protein, 108 aa, Mus musculus
  light chain variable region of PD-1 antibody
DIVLTQTPAIMSASPGEKVTLTCSASSSVSSNYLYWYQQRPGSSPKLWIYSTSNLASGVPARFSGSGSGTSYSLTISSMEAEDAASYFCHQWSSYPPTFGSGTKLEIK

SEQ ID NO: 86 - protein, 108 aa, Mus musculus
  light chain variable region of PD-1 antibody
DIVITQTTAIMSASPGEKVTLTCSASSSVSSNYLYWYQQRPGSSPKLWIYSTSNLASGVPARFSGSGSGTSYSLTISSMEAEDAASYFCHQWSSYPPTFGSGTKLEIK

SEQ ID NO: 87 - protein, 108 aa, Mus musculus
  light chain variable region of PD-1 antibody
DIVMTQTPATMSASPGEKVTLTCSASSSVNSNYLYWYQQKPGSSPKVWIYSTSNLASGVPARFSGSGSGTSYSLTISSMEAEDAASYFCHQWSSYPPTFGSGTKLELK

SEQ ID NO: 88 - protein, 108 aa, Mus musculus
  light chain variable region of PD-1 antibody
DIVMTQTTATMSASPGEKVTLTCSASSSVNSNYLYWYQQKPGSSPKVWIYSTSNLASGVPARFSGSGSGTSYSLTISSMEAEDAASYFCHQWSSYPPTFGSGTKLELK

SEQ ID NO: 89 - protein, 108 aa, Mus musculus
  light chain variable region of PD-1 antibody
DIVLTQSTAIMSASPGEKVTLTCSASSGVNSNYLYWYQQKPGSSPKLWIYSTSNLASGVPARFSGSGSGTSYSLTISSVEAEDAASYFCHQWSSYPPTFGSGTKLEIK

SEQ ID NO: 90 - protein, 107 aa, Mus musculus
  light chain variable region of PD-1 antibody
DIVLTQTPSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 91 - protein, 107 aa, Mus musculus
  light chain variable region of PD-1 antibody
DIVLTQSPSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 92 - protein, 107 aa, Mus musculus
  light chain variable region of PD-1 antibody
DIVITQSPSSLSASLGDRVTISCSASQGISNYLNWYQQKPDGTVKLLIYYTSSLHSGVPSRFSGSGSGTDYSLTISNLEPEDIATYYCQQYSELPWTFGGGTKLEIK

SEQ ID NO: 93 - protein, 107 aa, Mus musculus
  light chain variable region of PD-1 antibody
DIVMTQSPSSLSASLGDRVTISCSASQGISNYLNWYQQRPDGTVKLLIYYTSSLHSGVPSRFSGSGSGTDYSLTISNLEPEDIATYYCQQYSNLPWTFGGGTKLEIK

SEQ ID NO: 94 - protein, 107 aa, Mus musculus
  light chain variable region of PD-1 antibody
DIVMTQSPSSLSASLGDRVTISCSASQGISNYLNWYQQKPDGTVKLLIYYTSSLHSGVPSRFSGSGSGTDYSLTISNLEPEDIATYYCQQYSELPWTFGGGTKLEIK

SEQ ID NO: 95 - protein, 107 aa, Mus musculus
  light chain variable region of PD-1 antibody
DIVMTQSTSSLSASLGDRVTISCSASQGISHYLNWYQQKPDGTVKLLIYYTSSLHSGVPSRFSGSGSGTDYSLTIRNLEPEDIATYYCQQYSELPWTFGGGTKLEIK

SEQ ID NO: 96 - protein, 107 aa, Mus musculus
  light chain variable region of PD-1 antibody
DIVMTQSPSSLSASLGDRVTISCSASQGISHYLNWYQQKPDGTVKLLIYYTSSLHSGVPSRFSGSGSGTDYSLTIRNLEPEDIATYYCQQYSELPWTFGGGTKLEIK

SEQ ID NO: 97 - protein, 107 aa, Mus musculus
  light chain variable region of PD-1 antibody
DIVMTQSPSSLSASLGDRVTISCSASQDISSYLNWYQQKPDGTVKLLIYYTSSLHSGVPSRFSGSGSGTDYSLTISNLEPEDIATYYCQQYSELPWTFGGGTKLEIK

SEQ ID NO: 98 - protein, 107 aa, Mus musculus
  light chain variable region of PD-1 antibody
DIVMTQTPSSLSASLGDRVTISCSASQGISYYLNWYQQKPDGTIKLLIYYTLSLHSGVPSRFSGSGSGTDYSLTISNLEPEDIATYYCQQYSELPWTFGGGTKLEIK

SEQ ID NO: 99 - protein, 107 aa, Mus musculus
  light chain variable region of PD-1 antibody
DIVMTQTPSSMSASLGDRVTISCSASQGISNYLNWYQQKPDGTVKLLIYYTSSLHSGVPSRFSGSGSGTDYSLTISNLEPEDIATYYCQQYSYLPWTFGGGTKLEIK

SEQ ID NO: 100 - protein, 107 aa, Mus musculus
  light chain variable region of PD-1 antibody
DIVMTQTPSSLSASLGDRVTISCSASQGIGNYLNWYQQKPDGTVKLLIYYTSNLHSGVPSRFSGRGSGTDYSLTISNLEPEDIATYYCQQYSNLPWTFGGGTKLEIK

SEQ ID NO: 101 - protein, 107 aa, Mus musculus
  light chain variable region of PD-1 antibody
DIVMTQSPSSLSASLGDRVTISCSASQGISNYLNWYQQKPDGTVKLLIYYTSNLHSGVPSRFSGSGSGTDYSLTISDLAPEDIATYYCQQYSYLPWTFGGGTKLEIK

SEQ ID NO: 102 - protein, 112 aa, Mus musculus
  light chain variable region of PD-1 antibody
DIVITQSPLSLPVGLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 103 - protein, 112 aa, Mus musculus
  light chain variable region of PD-1 antibody
DIVLTQTPLSLPVSLGDQASISCRSSQSIVHSNGNTYLEWYLQKPGQSPKLLIYKVSNRFSGVPDRFSGSGSGTNFTLKISRVEAEDLGVYYCFQGSHVPLTFGAGTKLELK

SEQ ID NO: 104 - protein, 112 aa, Mus musculus
  light chain variable region of PD-1 antibody
DIVLTQSPLSLPVSLGDQASISCRSSQSIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 105 - protein, 112 aa, Mus musculus
  light chain variable region of PD-1 antibody
DIVITQTPLSLPVSLGDQASISCRSSQTIVHSDGNTYLEWYLQKPGQSPKLLIYKVSNRFSGVPDRFSGSGSGTDFTLKISRVEAEDLGVYYCFQGSHVPLTFGAGTKLEIK

SEQ ID NO: 106 - protein, 112 aa, Mus musculus
  light chain variable region of PD-1 antibody
DIVMTQSTLSLPVSLGDQVSISCRSSQSIVHSDGNTYLEWYLQKPGQSPNLLIYKVSNRFSGVPDRFSGSGSGTDFTLKISRVEAEDLGVYYCFQGSHVPLTFGAGTKLELK

SEQ ID NO: 107 - protein, 112 aa, Mus musculus
  light chain variable region of PD-1 antibody
DIVLTQDELSNPVTSGESVSISCRSSKSLLYKDGKTYLNWFLQRPGQSPQVLIYFMSTRASGVSDRFSGSGSGTDFTLEISRVKAEDVGVYYCQQLVDFPFTFGSGTKLELK

SEQ ID NO: 108 - protein, 112 aa, Mus musculus
  light chain variable region of PD-1 antibody
DIVMTQDELYNPVTSGESVSISCRSSKSLLYKDGKTYLNWFLQRPGQSPQVLIYFMSTRASGVSDRFSGSGSGTDFTLEISRVKAEDVGVYYCQQLVDFPFTFGSGTKLEIK

SEQ ID NO: 109 - protein, 113 aa, Mus musculus
  light chain variable region of PD-1 antibody
DIVMTQSPSSLTVTAGEKVTMSCKSSQSLLNSGTQKNYLTWYQQKPGQPPKLLIYWASTRESGVPDRFTGSGSGTDFTLTISSVQAEDLAVYYCQNDYSYPLTFGAGTKLEIK

SEQ ID NO: 110 - protein, 107 aa, Mus musculus
  light chain variable region of PD-1 antibody
DIVLTQTTATLSVTPGDRVSLSCRASQSISDYLHWYQQKSHESPRLLIKYASQSISGIPSRFSGSGSGSDFTLTVNSVEPEDVGVYYCQNGHSYPYTFGGGTKLEIK

SEQ ID NO: 111 - protein, 107 aa, Mus musculus
  light chain variable region of PD-1 antibody
DIVLTQSPDTLSVTPGDRVSLSCRASQSISDYLHWYQQKSHESPRLLIKYASQSISGIPSRFSGSGSGSDFTLSINSVEPEDVGVYYCQNGHSYPYTFGGGTKLELK

SEQ ID NO: 112 - protein, 120 aa, Artificial Sequence
  heavy chain variable region of PD-1 antibody
QVQLVQSGAEVKKPGASVKVSCKASGYTFTSYWMHWVRQAPGQGLEWMGMIDPSNSETSLNQKFQGRVTMTVDKSTNTVYMELSSLRSEDTAVYYCARSRGNYAYEMDYWGQGTLVTVSS

SEQ ID NO: 113 - protein, 120 aa, Artificial Sequence
  heavy chain variable region of PD-1 antibody
EVQLVQSGAEVKKPGASVKVSCKASGYTFTSYWMHWVRQAPGQGLEWMGMIDPSNSETSLNQKFQGRVTLNVDKSTNTAYMELSSLRSEDTAVYYCARSRGNYAYEMDYWGQGTLVTVSS

SEQ ID NO: 114 - protein, 120 aa, Artificial Sequence
  heavy chain variable region of PD-1 antibody
EVQLVQSGTEVTKPGASVKVSCKASGYTFTSYWMHWVRQAPGQGLEWLGMIDPSNSETTLNQKFQGRVTMTVDKSTNTVYMELTSLRSEDTAVYYCARSRGNYAYEMDYWGQGTLVTVSS

SEQ ID NO: 115 - protein, 120 aa, Artificial Sequence
  heavy chain variable region of PD-1 antibody
EVQLVQSGAEVKKPGASVKVSCKASGYTFTDYEMHWVRQAPGQGLEWMGIIDPGTGGTAYNQKFQGRVTMTADKSTSTVYMELSSLRSEDTAVYYCTSEKFGSNYYFDYWGQGTLVTVSS

SEQ ID NO: 116 - protein, 120 aa, Artificial Sequence
  heavy chain variable region of PD-1 antibody
EVQLVQSGAEVKKPGASVKVSCKASGYTFTDYEMHWVRQAPGQGLEWMGIIDPGTGGTAYNQKFQGRVTMTADKSTNTVYMELSSLRSEDTAVYYCTSEKFGSNYYFDYWGQGTLVTVSS

SEQ ID NO: 117 - protein, 120 aa, Artificial Sequence
  heavy chain variable region of PD-1 antibody
EVQLVQSGAEVKKPGASVKVSCKASGYTFTDYEMHWVRQAPGQRLEWMGVIDPGTGGTAYNQKFQGRVTITADKSASTAYMELSSLRSEDTAVYYCTSEKFGSNYYFDYWGQGTLVTVSS

SEQ ID NO: 118 - protein, 108 aa, Artificial Sequence
  light chain variable region of PD-1 antibody
EIVLTQSPATLSLSPGERATLSCRASSSVSSNYLYWYQQKPGQAPRLLIYSTSNRATGIPARFSGSGSGTDYTLTISSLEPEDFAVYYCHQWSSYPPTFGQGTKLEIK

SEQ ID NO: 119 - protein, 108 aa, Artificial Sequence
  light chain variable region of PD-1 antibody
DIVLTQSPATLSLSPGERATLSCRASSSVSSNYLYWYQQKPGQAPRLLIYSTSNLATGIPARFSGSGSGTDYTLTISSLEPEDFAVYFCHQWSSYPPTFGQGTKLEIK

SEQ ID NO: 120 - protein, 108 aa, Artificial Sequence
  light chain variable region of PD-1 antibody
DIVLTQSPGTLSLSPGEKVTLSCRASSSVSSNYLYWYQQKPGQAPRLVIYSTSNLATGIPDRFSGSGSGTDYTLTISRLEPEDFAVYFCHQWSSYPPTFGQGTKVEIK

SEQ ID NO: 121 - protein, 112 aa, Artificial Sequence
  light chain variable region of PD-1 antibody
DVVMTQSPLSLPVTLGQPASISCRSSQTIVHSDGNTYLEWYQQRPGQSPRLLIYKVSNRFSGVPDRFSGSGSGTDFTLKISRVEAEDVGVYYCFQGSHVPLTFGQGTKLEIK

SEQ ID NO: 122 - protein, 112 aa, Artificial Sequence
  light chain variable region of PD-1 antibody
DIVMTQSPLSLPVTLGQPASISCRSSQTIVHSDGNTYLEWYQQRPGQSPKLLIYKVSNRFSGVPDRFSGSGSGTDFTLKISRVEAEDVGVYYCFQGSHVPLTFGQGTKLEIK

SEQ ID NO: 123 - protein, 112 aa, Artificial Sequence
  light chain variable region of PD-1 antibody
DIVMTQTPLSSPVTLGQPASISCRSSQTIVHSDGNTYLEWYQQRPGQPPRLLIYKVSNRFSGVPDRFSGSGAGTDFTLKISRVEAEDVGVYYCFQGSHVPLTFGQGTKLEIK

SEQ ID NO: 124 - protein, 15 aa, Artificial Sequence
  PD-1 peptide sequence
MQIPQAPWPVVWAVL

SEQ ID NO: 125 - protein, 15 aa, Artificial Sequence
  PD-1 peptide sequence
APWPVVWAVLQLGWR

SEQ ID NO: 126 - protein, 15 aa, Artificial Sequence
  PD-1 peptide sequence
VWAVLQLGWRPGWFL

SEQ ID NO: 127 - protein, 15 aa, Artificial Sequence
  PD-1 peptide sequence
QLGWRPGWFLDSPDR

SEQ ID NO: 128 - protein, 15 aa, Artificial Sequence
  PD-1 peptide sequence
PGWFLDSPDRPWNPP

SEQ ID NO: 129 - protein, 15 aa, Artificial Sequence
  PD-1 peptide sequence
DSPDRPWNPPTFSPA

SEQ ID NO: 130 - protein, 15 aa, Artificial Sequence
  PD-1 peptide sequence
PWNPPTFSPALLVVT

SEQ ID NO: 131 - protein, 15 aa, Artificial Sequence
  PD-1 peptide sequence
TFSPALLVVTEGDNA

SEQ ID NO: 132 - protein, 15 aa, Artificial Sequence
  PD-1 peptide sequence
LLVVTEGDNATFTCS

SEQ ID NO: 133 - protein, 15 aa, Artificial Sequence
  PD-1 peptide sequence
EGDNATFTCSFSNTS

SEQ ID NO: 134 - protein, 15 aa, Artificial Sequence
  PD-1 peptide sequence
TFTCSFSNTSESFVL

SEQ ID NO: 135 - protein, 15 aa, Artificial Sequence
  PD-1 peptide sequence
FSNTSESFVLNWYRM

SEQ ID NO: 136 - protein, 15 aa, Artificial Sequence
  PD-1 peptide sequence
ESFVLNWYRMSPSNQ

SEQ ID NO: 137 - protein, 15 aa, Artificial Sequence
  PD-1 peptide sequence
NWYRMSPSNQTDKLA

SEQ ID NO: 138 - protein, 15 aa, Artificial Sequence
  PD-1 peptide sequence
SPSNQTDKLAAFPED

SEQ ID NO: 139 - protein, 15 aa, Artificial Sequence
  PD-1 peptide sequence
TDKLAAFPEDRSQPG

SEQ ID NO: 140 - protein, 15 aa, Artificial Sequence
  PD-1 peptide sequence
AFPEDRSQPGQDCRF

SEQ ID NO: 141 - protein, 15 aa, Artificial Sequence
  PD-1 peptide sequence
RSQPGQDCRFRVTQL

SEQ ID NO: 142 - protein, 15 aa, Artificial Sequence
  PD-1 peptide sequence
QDCRFRVTQLPNGRD

SEQ ID NO: 143 - protein, 15 aa, Artificial Sequence
  PD-1 peptide sequence
RVTQLPNGRDFHMSV

SEQ ID NO: 144 - protein, 15 aa, Artificial Sequence
  PD-1 peptide sequence
PNGRDFHMSVVRARR

SEQ ID NO: 145 - protein, 15 aa, Artificial Sequence
  PD-1 peptide sequence
FHMSVVRARRNDSGT

SEQ ID NO: 146 - protein, 15 aa, Artificial Sequence
  PD-1 peptide sequence
VRARRNDSGTYLCGA

SEQ ID NO: 147 - protein, 15 aa, Artificial Sequence
  PD-1 peptide sequence
NDSGTYLCGAISLAP

SEQ ID NO: 148 - protein, 15 aa, Artificial Sequence
  PD-1 peptide sequence
YLCGAISLAPKAQIK

SEQ ID NO: 149 - protein, 15 aa, Artificial Sequence
  PD-1 peptide sequence
ISLAPKAQIKESLRA

SEQ ID NO: 150 - protein, 15 aa, Artificial Sequence
  PD-1 peptide sequence
KAQIKESLRAELRVT

SEQ ID NO: 151 - protein, 15 aa, Artificial Sequence
  PD-1 peptide sequence
ESLRAELRVTERRAE

SEQ ID NO: 152 - protein, 15 aa, Artificial Sequence
  PD-1 peptide sequence
ELRVTERRAEVPTAH

SEQ ID NO: 153 - protein, 15 aa, Artificial Sequence
  PD-1 peptide sequence
ERRAEVPTAHPSPSP

SEQ ID NO: 154 - protein, 16 aa, Artificial Sequence
  PD-1 peptide sequence
VPTAHPSPSPRPAGQF

SEQ ID NO: 155 - protein, 773 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 156 - protein, 773 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 157 - protein, 548 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 158 - protein, 548 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 159 - protein, 781 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 160 - protein, 781 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 161 - protein, 552 aa, Artificial Sequence
  bispecific fusion polypeptide
ASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVSGTWYLKAMTVDSDCFWIDDVSVTPMTLTTLEGGNLEAKVTMDIFGFWQEVKAVLEKTDEPGKYTADGGKHVAYIIRSHVKDHYIFYSEGECAGYPVPGVWLVGRDPKNNLEALEDFEKAAGARGLSTESILIPRQSETSSPG

SEQ ID NO: 162 - protein, 552 aa, Artificial Sequence
  bispecific fusion polypeptide
ASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPGGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSASDEEIQDVPGTWYLKAMTVSGEDPEMMLESVTPMTLTTLEGGNLEARVTVLIDGRCQEVKNVLEKTDEPGKYTEDGGKHVDYIIRSHVKDHYIFYFEGEGQGTPGRMVALVGRDPTNNLEALEDFEKAAGARGLSTESILIPRQSETCSPG

SEQ ID NO: 163 - protein, 8 aa, Artificial Sequence
  VH-CDR1 of anti-PD1 antibody
GYTFTDYE

SEQ ID NO: 164 - protein, 8 aa, Artificial Sequence
  VH-CDR2 of anti-PD1 antibody
IDPGTGGT

SEQ ID NO: 165 - protein, 13 aa, Artificial Sequence
  VH-CDR3 of anti-PD1 antibody
TSEKFGSNYYFDY

SEQ ID NO: 166 - protein, 11 aa, Artificial Sequence
  VL-CDR1 of anti-PD1 antibody
QTIVHSDGNTY

SEQ ID NO: 167 - protein, 9 aa, Artificial Sequence
  VL-CDR3 of anti-PD1 antibody
FQGSHVPLT

SEQ ID NO: 168 - protein, 8 aa, Artificial Sequence
  VH-CDR1 of anti-PD1 antibody
GYTFTSYW

SEQ ID NO: 169 - protein, 8 aa, Artificial Sequence
  VH-CDR2 of anti-PD1 antibody
IDPSNSET

SEQ ID NO: 170 - protein, 13 aa, Artificial Sequence
  VH-CDR3 of anti-PD1 antibody
ARSRGNYAYEMDY

SEQ ID NO: 171 - protein, 7 aa, Artificial Sequence
  VL-CDR1 of anti-PD1 antibody
SSVSSNY

SEQ ID NO: 172 - protein, 8 aa, Artificial Sequence
  VL-CDR3 of anti-PD1 antibody
HQWSSYPP

SEQ ID NO: 173 - protein, 8 aa, Artificial Sequence
  VH-CDR1 of anti-PD1 antibody
GYTFTDYW

SEQ ID NO: 174 - protein, 8 aa, Artificial Sequence
  VH-CDR2 of anti-PD1 antibody
IDTSDSYT

SEQ ID NO: 175 - protein, 10 aa, Artificial Sequence
  VH-CDR3 of anti-PD1 antibody
ARRDYGGFGY

SEQ ID NO: 176 - protein, 6 aa, Artificial Sequence
  VL-CDR1 of anti-PD1 antibody
QDISSY

SEQ ID NO: 177 - protein, 8 aa, Artificial Sequence
  VL-CDR3 of anti-PD1 antibody
QQYSELPW

SEQ ID NO: 178 - protein, 8 aa, Artificial Sequence
  VH-CDR1 of anti-PD1 antibody
GYTFTDYN

SEQ ID NO: 179 - protein, 8 aa, Artificial Sequence
  VH-CDR2 of anti-PD1 antibody
IDPNNGDT

SEQ ID NO: 180 - protein, 9 aa, Artificial Sequence
  VH-CDR3 of anti-PD1 antibody
ARWRSSMDY

SEQ ID NO: 181 - protein, 6 aa, Artificial Sequence
  VL-CDR1 of anti-PD1 antibody
QGISNY

SEQ ID NO: 182 - protein, 8 aa, Artificial Sequence
  VL-CDR3 of anti-PD1 antibody
QQYSNLPW

SEQ ID NO: 183 - protein, 9 aa, Artificial Sequence
  VH-CDR1 of anti-PD1 antibody
GYSITSDYA

SEQ ID NO: 184 - protein, 7 aa, Artificial Sequence
  VH-CDR2 of anti-PD1 antibody
ITYSGSP

SEQ ID NO: 185 - protein, 11 aa, Artificial Sequence
  VH-CDR3 of anti-PD1 antibody
ARGLGGHYFDY

SEQ ID NO: 186 - protein, 6 aa, Artificial Sequence
  VL-CDR1 of anti-PD1 antibody
QSISDY

SEQ ID NO: 187 - protein, 8 aa, Artificial Sequence
  VL-CDR3 of anti-PD1 antibody
QNGRSYPY

SEQ ID NO: 188 - protein, 8 aa, Artificial Sequence
  VH-CDR1 of anti-PD1 antibody
GFSLTSYG

SEQ ID NO: 189 - protein, 7 aa, Artificial Sequence
  VH-CDR2 of anti-PD1 antibody
IWRGGNT

SEQ ID NO: 190 - protein, 8 aa, Artificial Sequence
  VH-CDR3 of anti-PD1 antibody
AASMIGGY

SEQ ID NO: 191 - protein, 11 aa, Artificial Sequence
  VL-CDR1 of anti-PD1 antibody
QSIVHSNGNTY

SEQ ID NO: 192 - protein, 8 aa, Artificial Sequence
  VL-CDR3 of anti-PD1 antibody
FQGSHVPL